SEQUENCE LISTING (78 sequences)

SEQ ID NO: 1 - protein, 38 aa, Homo sapiens
  MISC_FEATURE (38)..(38): Sequence has an amidated C-terminus
HSDGIFTDSYSRYRKQMAVKKYLAAVLGKRYKQRVKNK

SEQ ID NO: 2 - protein, 27 aa, Homo sapiens
  MISC_FEATURE (27)..(27): Sequence has an amidated C-terminus
HSDGIFTDSYSRYRKQMAVKKYLAAVL

SEQ ID NO: 3 - protein, 28 aa, Homo sapiens
  MISC_FEATURE (28)..(28): Sequence has an amidated C-terminus
HSDAVFTDNYTRLRKQMAVKKYLNSILN

SEQ ID NO: 4 - protein, 38 aa, Artificial Sequence
  Synthetic Construct
XSDGIFTDSYSRYRKQMAVKKYLAAVLGKRYKQRVKNK

SEQ ID NO: 5 - protein, 38 aa, Artificial Sequence; identical to SEQ ID NO: 4
  Synthetic Construct

SEQ ID NO: 6 - protein, 38 aa, Artificial Sequence
  Synthetic Construct
XSDGIFTDSYSRYRKAAAVKKYLAAVLGKRYKQRVKNX

SEQ ID NO: 7 - protein, 38 aa, Artificial Sequence
  Synthetic Construct
XXDGIFTDSYSRYRKQMAVKKYLAAVLGKRYKQRVKNK

SEQ ID NO: 8 - protein, 38 aa, Artificial Sequence
  Synthetic Construct
XSDGIFTDSYSRYRKQMAVKKALAAVLGKRYKQRVKNK

SEQ ID NO: 9 - protein, 38 aa, Artificial Sequence; identical to SEQ ID NO: 8
  Synthetic Construct

SEQ ID NO: 10 - protein, 38 aa, Artificial Sequence; identical to SEQ ID NO: 8
  Synthetic Construct

SEQ ID NO: 11 - protein, 38 aa, Artificial Sequence
  Synthetic Construct
XXDGIFTDSYSRYRKQMAVKKALAAVLGKRYKQRVKNK

SEQ ID NO: 12 - protein, 38 aa, Artificial Sequence; identical to SEQ ID NO: 11
  Synthetic Construct

SEQ ID NO: 13 - protein, 38 aa, Artificial Sequence; identical to SEQ ID NO: 11
  Synthetic Construct

SEQ ID NO: 14 - protein, 38 aa, Artificial Sequence; identical to SEQ ID NO: 11
  Synthetic Construct

SEQ ID NO: 15 - protein, 39 aa, Artificial Sequence
  Synthetic Construct
XXXXXXXXXXXXXXXXXXXXXXXXXXXXXXXXXXXXXXX

SEQ ID NO: 16 - protein, 38 aa, Artificial Sequence; identical to SEQ ID NO: 4
  Synthetic Construct

SEQ ID NO: 17 - protein, 38 aa, Artificial Sequence
  Synthetic Construct
HXDGIFTDSYSRYRKQMAVKKYLAAVLGKRYKQRVKNK

SEQ ID NO: 18 - protein, 38 aa, Artificial Sequence
  Synthetic Construct
HSDGIFTDSYSRYRKXMAVKKYLAAVLGKRYKQRVKNK

SEQ ID NO: 19 - protein, 38 aa, Artificial Sequence
  Synthetic Construct
HSDGIFTDSYSRYRKQXAVKKYLAAVLGKRYKQRVKNK

SEQ ID NO: 20 - protein, 38 aa, Artificial Sequence
  Synthetic Construct
HSDGIFTDSYSRYRKQMAVKKXLAAVLGKRYKQRVKNK

SEQ ID NO: 21 - protein, 38 aa, Artificial Sequence
  Synthetic Construct
HSDGIFTDSYSRYRKQMAVKKYLAAVLGKRYKQRVKNX

SEQ ID NO: 22 - protein, 38 aa, Artificial Sequence; identical to SEQ ID NO: 7
  Synthetic Construct

SEQ ID NO: 23 - protein, 38 aa, Artificial Sequence
  Synthetic Construct
XSDGIFTDSYSRYRKXMAVKKYLAAVLGKRYKQRVKNK

SEQ ID NO: 24 - protein, 38 aa, Artificial Sequence
  Synthetic Construct
XSDGIFTDSYSRYRKQXAVKKYLAAVLGKRYKQRVKNK

SEQ ID NO: 25 - protein, 38 aa, Artificial Sequence
  Synthetic Construct
XSDGIFTDSYSRYRKQMAVKKXLAAVLGKRYKQRVKNK

SEQ ID NO: 26 - protein, 38 aa, Artificial Sequence
  Synthetic Construct
XSDGIFTDSYSRYRKQMAVKKYLAAVLGKRYKQRVKNX

SEQ ID NO: 27 - protein, 38 aa, Artificial Sequence
  Synthetic Construct
HXDGIFTDSYSRYRKXMAVKKYLAAVLGKRYKQRVKNK

SEQ ID NO: 28 - protein, 38 aa, Artificial Sequence
  Synthetic Construct
HXDGIFTDSYSRYRKQXAVKKYLAAVLGKRYKQRVKNK

SEQ ID NO: 29 - protein, 38 aa, Artificial Sequence
  Synthetic Construct
HXDGIFTDSYSRYRKQMAVKKXLAAVLGKRYKQRVKNK

SEQ ID NO: 30 - protein, 38 aa, Artificial Sequence
  Synthetic Construct
HXDGIFTDSYSRYRKQMAVKKYLAAVLGKRYKQRVKNX

SEQ ID NO: 31 - protein, 38 aa, Artificial Sequence
  Synthetic Construct
HSDGIFTDSYSRYRKXXAVKKYLAAVLGKRYKQRVKNK

SEQ ID NO: 32 - protein, 38 aa, Artificial Sequence
  Synthetic Construct
HSDGIFTDSYSRYRKXMAVKKXLAAVLGKRYKQRVKNK

SEQ ID NO: 33 - protein, 38 aa, Artificial Sequence
  Synthetic Construct
HSDGIFTDSYSRYRKXMAVKKYLAAVLGKRYKQRVKNX

SEQ ID NO: 34 - protein, 38 aa, Artificial Sequence
  Synthetic Construct
HSDGIFTDSYSRYRKQXAVKKXLAAVLGKRYKQRVKNK

SEQ ID NO: 35 - protein, 38 aa, Artificial Sequence
  Synthetic Construct
HSDGIFTDSYSRYRKQXAVKKYLAAVLGKRYKQRVKNX

SEQ ID NO: 36 - protein, 38 aa, Artificial Sequence
  Synthetic Construct
HSDGIFTDSYSRYRKQMAVKKXLAAVLGKRYKQRVKNX

SEQ ID NO: 37 - protein, 38 aa, Artificial Sequence
  Synthetic Construct
XXDGIFTDSYSRYRKXMAVKKYLAAVLGKRYKQRVKNK

SEQ ID NO: 38 - protein, 38 aa, Artificial Sequence
  Synthetic Construct
XXDGIFTDSYSRYRKQXAVKKYLAAVLGKRYKQRVKNK

SEQ ID NO: 39 - protein, 38 aa, Artificial Sequence
  Synthetic Construct
XXDGIFTDSYSRYRKQMAVKKXLAAVLGKRYKQRVKNK

SEQ ID NO: 40 - protein, 38 aa, Artificial Sequence
  Synthetic Construct
XXDGIFTDSYSRYRKQMAVKKYLAAVLGKRYKQRVKNX

SEQ ID NO: 41 - protein, 38 aa, Artificial Sequence
  Synthetic Construct
XSDGIFTDSYSRYRKXXAVKKYLAAVLGKRYKQRVKNK

SEQ ID NO: 42 - protein, 38 aa, Artificial Sequence
  Synthetic Construct
XSDGIFTDSYSRYRKXMAVKKXLAAVLGKRYKQRVKNK

SEQ ID NO: 43 - protein, 38 aa, Artificial Sequence
  Synthetic Construct
XSDGIFTDSYSRYRKXMAVKKYLAAVLGKRYKQRVKNX

SEQ ID NO: 44 - protein, 38 aa, Artificial Sequence
  Synthetic Construct
XSDGIFTDSYSRYRKQXAVKKXLAAVLGKRYKQRVKNK

SEQ ID NO: 45 - protein, 38 aa, Artificial Sequence
  Synthetic Construct
XSDGIFTDSYSRYRKQXAVKKYLAAVLGKRYKQRVKNX

SEQ ID NO: 46 - protein, 38 aa, Artificial Sequence
  Synthetic Construct
XSDGIFTDSYSRYRKQMAVKKXLAAVLGKRYKQRVKNX

SEQ ID NO: 47 - protein, 38 aa, Artificial Sequence
  Synthetic Construct
HXDGIFTDSYSRYRKXXAVKKYLAAVLGKRYKQRVKNK

SEQ ID NO: 48 - protein, 38 aa, Artificial Sequence
  Synthetic Construct
HXDGIFTDSYSRYRKXMAVKKXLAAVLGKRYKQRVKNK

SEQ ID NO: 49 - protein, 38 aa, Artificial Sequence
  Synthetic Construct
HXDGIFTDSYSRYRKXMAVKKYLAAVLGKRYKQRVKNX

SEQ ID NO: 50 - protein, 38 aa, Artificial Sequence
  Synthetic Construct
HXDGIFTDSYSRYRKQXAVKKXLAAVLGKRYKQRVKNK

SEQ ID NO: 51 - protein, 38 aa, Artificial Sequence
  Synthetic Construct
HXDGIFTDSYSRYRKQXAVKKYLAAVLGKRYKQRVKNX

SEQ ID NO: 52 - protein, 38 aa, Artificial Sequence
  Synthetic Construct
HXDGIFTDSYSRYRKQMAVKKXLAAVLGKRYKQRVKNX

SEQ ID NO: 53 - protein, 38 aa, Artificial Sequence
  Synthetic Construct
HSDGIFTDSYSRYRKXXAVKKXLAAVLGKRYKQRVKNK

SEQ ID NO: 54 - protein, 38 aa, Artificial Sequence
  Synthetic Construct
HSDGIFTDSYSRYRKXXAVKKYLAAVLGKRYKQRVKNX

SEQ ID NO: 55 - protein, 38 aa, Artificial Sequence; identical to SEQ ID NO: 1
  Synthetic Construct

SEQ ID NO: 56 - protein, 38 aa, Artificial Sequence
  Synthetic Construct
HSDGIFTDSYSRYRKQXAVKKXLAAVLGKRYKQRVKNX

SEQ ID NO: 57 - protein, 38 aa, Artificial Sequence
  Syntehtic Construct
XXDGIFTDSYSRYRKXXAVKKYLAAVLGKRYKQRVKNK

SEQ ID NO: 58 - protein, 38 aa, Artificial Sequence
  Synthetic Construct
XXDGIFTDSYSRYRKXMAVKKXLAAVLGKRYKQRVKNK

SEQ ID NO: 59 - protein, 38 aa, Artificial Sequence
  Synthetic Construct
XXDGIFTDSYSRYRKXMAVKKYLAAVLGKRYKQRVKNX

SEQ ID NO: 60 - protein, 38 aa, Artificial Sequence
  Synthetic Construct
XXDGIFTDSYSRYRKQXAVKKXLAAVLGKRYKQRVKNK

SEQ ID NO: 61 - protein, 38 aa, Artificial Sequence
  Synthetic Construct
XXDGIFTDSYSRYRKQXAVKKYLAAVLGKRYKQRVKNX

SEQ ID NO: 62 - protein, 38 aa, Artificial Sequence
  Synthetic Construct
XXDGIFTDSYSRYRKQMAVKKXLAAVLGKRYKQRVKNX

SEQ ID NO: 63 - protein, 38 aa, Artificial Sequence
  Synthetic Construct
XSDGIFTDSYSRYRKXXAVKKXLAAVLGKRYKQRVKNK

SEQ ID NO: 64 - protein, 38 aa, Artificial Sequence
  Synthetic Construct
XSDGIFTDSYSRYRKXXAVKKYLAAVLGKRYKQRVKNX

SEQ ID NO: 65 - protein, 38 aa, Artificial Sequence
  Synthetic Construct
XSDGIFTDSYSRYRKXMAVKKXLAAVLGKRYKQRVKNX

SEQ ID NO: 66 - protein, 38 aa, Artificial Sequence
  Synthetic Construct
XSDGIFTDSYSRYRKQXAVKKXLAAVLGKRYKQRVKNX

SEQ ID NO: 67 - protein, 38 aa, Artificial Sequence
  Synthetic Construct
HXDGIFTDSYSRYRKXXAVKKXLAAVLGKRYKQRVKNK

SEQ ID NO: 68 - protein, 38 aa, Artificial Sequence
  Synthetic Construct
HXDGIFTDSYSRYRKXXAVKKYLAAVLGKRYKQRVKNX

SEQ ID NO: 69 - protein, 38 aa, Artificial Sequence
  Synthetic Construct
HXDGIFTDSYSRYRKXMAVKKXLAAVLGKRYKQRVKNX

SEQ ID NO: 70 - protein, 38 aa, Artificial Sequence
  Synthetic Construct
HXDGIFTDSYSRYRKQXAVKKXLAAVLGKRYKQRVKNX

SEQ ID NO: 71 - protein, 38 aa, Artificial Sequence
  Synthetic Construct
HSDGIFTDSYSRYRKXXAVKKXLAAVLGKRYKQRVKNX

SEQ ID NO: 72 - protein, 38 aa, Artificial Sequence
  Synthetic Construct
XXDGIFTDSYSRYRKXXAVKKXLAAVLGKRYKQRVKNK

SEQ ID NO: 73 - protein, 38 aa, Artificial Sequence
  Synthetic Construct
XXDGIFTDSYSRYRKXXAVKKYLAAVLGKRYKQRVKNX

SEQ ID NO: 74 - protein, 38 aa, Artificial Sequence
  Synthetic Construct
XXDGIFTDSYSRYRKXMAVKKXLAAVLGKRYKQRVKNX

SEQ ID NO: 75 - protein, 38 aa, Artificial Sequence
  Synthetic Construct
XXDGIFTDSYSRYRKQXAVKKXLAAVLGKRYKQRVKNX

SEQ ID NO: 76 - protein, 38 aa, Artificial Sequence
  Synthetic Construct
XSDGIFTDSYSRYRKXXAVKKXLAAVLGKRYKQRVKNX

SEQ ID NO: 77 - protein, 38 aa, Artificial Sequence
  Synthetic Construct
HXDGIFTDSYSRYRKXXAVKKXLAAVLGKRYKQRVKNX

SEQ ID NO: 78 - protein, 38 aa, Artificial Sequence
  Synthetic Construct
XXDGIFTDSYSRYRKXXAVKKXLAAVLGKRYKQRVKNX

(19) United States
(12) Patent Application Publication
(10) Pub. No.: US 2016/0122406 A1
(43) Pub. Date: May 5, 2016

(54) ANALOGS OF PITUITARY ADENYLATE CYCLASE-ACTIVATING POLYPEPTIDE (PACAP) AND METHODS FOR THEIR USE

(71) Applicant: THE ADMINISTRATORS OF THE TULANE EDUCATIONAL FUND, New Orleans, LA (US)

(72) Inventors: David H. COY, New Orleans, LA (US); Jerome L. MADERDRUT, New Orleans, LA (US)

(21) Appl. No.: 14/896,178

(22) PCT Filed: Jun. 9, 2014
(86) PCT No.: PCT/US14/41597

§ 371 Date: Dec. 4, 2015

Related U.S. Application Data
(60) Provisional application No. 61/832,238, filed on Jun. 7, 2013.

(51) Int. Cl. C07K 14/47; A61K 51/08; A61K 49/00

(52) U.S. Cl. CPC C07K 14/4705; A61K 49/0004; A61K 51/08

(57) ABSTRACT

This invention relates to analogs of pituitary adenylate cyclase-activating polypeptide (PACAP), which are agonists for the PACAP/vasoactive intestinal peptide (VIP) receptors: PAC₁, VPAC₁ and VPAC₂ receptors. These PACAP analogs can be used as prophylactic/therapeutic agents for a wide range of medical disorders. These PACAP analogs coupled to suitable radionuclides can be used in the localization, diagnosis and treatment of disseminated cancers and metastatic tumors, and coupled to small molecule therapeutics can be used as vectors for targeted drug delivery. This invention also provides pharmaceutical compositions of one or more PACAP analogs of the invention either alone or in combination with one or more other prophylactic/therapeutic agent.

### CROSS REFERENCE TO RELATED APPLICATIONS

This application claims the benefit of U.S. application No. 61/832,238 filed Jun. 7, 2013. The content of this application is incorporated herein by reference in its entirety.

### FIELD OF THE INVENTION

This invention relates to analogs of pituitary adenylate cyclase-activating polypeptide (PACAP), which are agonists for the PACAP/vasoactive intestinal peptide (VIP) receptors: PAC₁, VPAC₁ and VPAC₂ receptors. These PACAP analogs can be used as prophylactic/therapeutic agents for a wide range of medical disorders, including (but not limited to) age-related neurodegenerative diseases (such as Alzheimer's disease, Parkinson's disease and amyotrophic lateral sclerosis), injuries to the central nervous system caused by stroke, heart attack and blunt force trauma (such as concussions and spinal cord trauma), Huntington's disease and other CAG codon repeat expansion diseases, retinal diseases (such as ischemia/reperfusion injury, non-infectious uveitis, diabetic retinopathy, macular degeneration, and glaucoma), autoimmune diseases (such as rheumatoid arthritis, Crohn's disease, ulcerative colitis, scleroderma, Sjögren's disease, idiopathic membranous nephropathy, Goodpasture's disease, autoimmune hepatitis, autoimmune myocarditis, myasthenia gravis, multiple sclerosis, Guillain-Barré syndrome, type I diabetes, Hashimoto's thyroiditis, Graves' disease, pemphigus vulgaris, and systemic lupus erythematosus), graft-versus-host disease, keratoconjunctivitis sicca caused by aging, autoimmune diseases, corneal transplantation, or keratorefractive surgery, type II diabetes, sepsis caused by bacteria and/or viruses (including bacterial and viral toxins), acute and chronic cardiovascular diseases (such as myocardial infarction, atherosclerosis, restenosis, and drug-induced cardiomyopathy), acute and chronic renal diseases (such as ischemia/reperfusion injury, nephritis and drug-induced nephrotoxicity), genetic disorders caused by premature in-frame stop codons, acute and chronic pulmonary diseases (such as asthma, chronic obstructive pulmonary disease, cystic fibrosis, and pulmonary arterial hypertension), systemic hypertension, hematological cancers (such as leukemias, lymphomas and plasma cell dyscrasias), DNA damage caused by therapeutic agents and radiation, eating disorders, acute and chronic liver diseases (such as ischemia/reperfusion injury, hepatitis and fatty liver), osteoporosis, pre-eclampsia, cell and solid organ transplantation, cognitive disorders, AIDS dementia complex, and aging of the central nervous system. These PACAP analogs coupled to suitable radionuclides can be used in the localization, diagnosis and treatment of disseminated cancers and metastatic tumors, and coupled to small molecule therapeutics can be used as vectors for targeted drug delivery. This invention also provides pharmaceutical compositions of one or more PACAP analogs of the invention either alone or in combination with one or more other prophylactic/therapeutic agents.

### BACKGROUND OF THE INVENTION

Pituitary adenylate cyclase-activating polypeptide (PACAP) was isolated from ovine (sheep) hypothalami based on its ability to stimulate adenylate cyclase activity in rat anterior pituitary cell cultures (Miyata et al., Biochem Biophys Res Commun 164:567-574, 1989). PACAP exists as two α-amidated peptides with 38 (PACAP38; SEQ ID NO:1) or 27 (PACAP27; SEQ ID NO:2) amino acids. Both peptides have the same N-terminal 27 amino acids and are synthesized from the same prohormone. The sequence of PACAP38 is identical in all mammals and differs from the avian and amphibian orthologs by only one amino acid (Vaudry et al., Pharmacol Rev 61:283-357, 2009). PACAP is a member of the secretin/vasoactive intestinal peptide (VIP)/growth hormone-releasing hormone (GHRH) family, and PACAP27 has 68% sequence identity with VIP (SEQ ID NO:3). PACAP is most abundant in the brain and testis, but there are significant levels in other organs, including the pancreas, adrenals, thymus, spleen, lymph nodes, and duodenal mucosa (Vaudry et al., Pharmacol Rev 61:283-357, 2009). PACAP is synthesized as a preprohormone and is processed mainly by prohormone convertase 1, prohormone convertase 2 and prohormone convertase 4 (Li et al., Neuroendocrinology 69:217-226, 1999; Li et al., Endocrinology 141:3723-3730, 2000). The half-life of [¹²⁵I]-PACAP38 in the bloodstream of rats following intravenous injection is 5-6 minutes (Banks et al., J Pharmacol Exp Ther 267:690-696, 1993). Members of the secretin/VIP/GHRH family are degraded in plasma mainly by aminodipeptidases, especially dipeptidyl peptidase IV (Zhu et al., J Biol Chem 278:22418-2223, 2003).

PACAP binds to three Class B (secretin-type) G-protein-coupled receptors that are called the PAC₁, VPAC₁ and VPAC₂ receptors (Harmar et al., Pharmacol Rev 50:265-270, 1998). Class B G-protein-coupled receptors have low sequence identity to Class A (rhodopsin-type) G-protein-coupled receptors. PACAP binds not only to the PAC₁ receptor with a high affinity, but it also binds to the VPAC₁ (VIP1) and VPAC₂ (VIP2) receptors with an affinity comparable to or greater than VIP. On the other hand, VIP binds to the PAC₁ receptor with an affinity 100-1,000 times less than PACAP (Arimura, Jpn J Physiol 48:301-331, 1998; Harmar et al., Br J Pharmacol 166:4-17, 2012). The PAC₁, VPAC₁ and VPAC₂ receptors have been cloned from several vertebrate species (Arimura, Jpn J Physiol 48:301-331, 1998; Vaudry et al., Pharmacol Rev 61:283-357, 2009). They are G-protein-coupled receptors with seven putative membrane-spanning domains and belong to a family of glycoprotein receptors that are coupled to multiple signal transduction pathways (Segre and Goldring, Trends Endocrinol Metab 4:309-314, 1993; Couvineau and Laburthe, Br J Pharmacol 166:42-50, 2012; Harmar et al., Br J Pharmacol 166:4-17, 2012). The “second” messengers include adenylate cyclase, phospholipase C, mitogen-activated protein (MAP) kinases, and calcium. At least 10 splice variants of the rat PAC₁ receptor have been cloned and each variant is coupled to distinct combinations of signal transduction pathways (Vaudry et al., Pharmacol Rev 61:283-357, 2009). PACAP/VIP receptor can be coupled to Gαs and/or Gαi in different types of cells. PACAP/VIP receptors are expressed in many different types of normal and cancer cells, including the catecholamine-containing cells in the adrenal medulla and the sympathetic ganglia; microglia, astrocytes and some types of neurons in the central nervous system; and T- and B-lymphocytes, macrophages, neutrophils, and dendritic cells in the immune system (Vaudry et al., Pharmacol Rev 61:283-357, 2009). PACAP is a potent stimulator of catecholamine secretion from the adrenal medulla (Watanabe et al., Am J Physiol 269:E903-E909, 1995; Smith and Eiden, J Mol Neurosci 48:403-412, 2012), but a potent inhibitor of the secretion of tumor necrosis factor-α (TNF-α), interleukin (IL)-6 and IL-12 from activated macrophages (Ganea and Delgado, Crit Rev Oral Biol Med 13:229-237, 2002). More pertinent to the present invention, PACAP stimulates the proliferation of C6 glioblastoma cells (Dufes et al., J Mol Neurosci 21:91-102, 2003), AR4-2J pancreatic carcinoma cells (Buscail et al., Gastroenterology 103:1002-1008, 1992), PC-3 and LNCaP prostate cancer cells (Leyton et al., Cancer Lett 125:131-139, 1998; cf. Juarranz et al., Prostate 47:285-292, 2001), NCI-N417 and NCI-H838 lung cancer cells (Moody et al., Peptides 14:241-246, 1993; Zia et al., Cancer Res 55:4886-4891, 1995), and MCF-7 and T47D breast cancer cells (Leyton et al., Breast Cancer Res Treat 56:177-186, 1999), but inhibits the proliferation of HEL myeloid leukemia cells (Hayez et al., J Neuroimmunol 149:167-181, 2004), Jurkat T-cell leukemia cells (Coy et al., PCT/US2011/023930, 2011) and multiple myeloma cells (Li et al., Regul Pept 145:24-32, 2008; Coy et al., PCT/US2010/055164, 2010). Small molecule orthosteric agonists have not been found for the cognate receptors for any member of the secretin/VIP/GHRH/PACAP family (Harmar et al., Br J Pharmacol 166:4-17, 2012; Hollenstein et al., Trends Pharmacol Sci 35:12-22, 2014).

Although PACAP was isolated during a screen for novel hypophysiotropic factors, it soon became clear that it is a pleiotropic peptide (Arimura, Jpn J Physiol 48:301-331, 1998; Vaudry et al., Pharmacol Rev 61:283-357, 2009). The extraordinarily potent neuroprotective/neurotrophic properties of PACAP were investigated by several laboratories shortly after its isolation. The cytoprotective effects of PACAP and VIP have been studied much more extensively in the nervous system than in any other major organ of the body. The cell types that were protected by PACAP in various in vitro models include cerebellar granule cells, dorsal root ganglion cells, sympathetic ganglion cells, mesencephalic dopaminergic neurons, and basal forebrain cholinergic neurons (Arimura, Jpn J Physiol 48:301-331, 1998; Vaudry et al., Pharmacol Rev 61:283-357, 2009). PACAP also prevented the neuronal death induced by gp120, the envelope glycoprotein of the human immunodeficiency virus (HIV), in rat hippocampal neuron/glia co-cultures. The dose-response curve was bimodal, with peaks at 10⁻¹³ M and 10⁻¹⁰ M (Arimura et al., Ann NY Acad Sci 739:228-243, 1994). The critical findings in this study have been confirmed by Kong et al. (Neuroscience 91:493-500, 1999), who used lipopolysaccharide as the neurotoxin in primary murine cortical neuron/glia co-cultures. The neuroprotective effect at 10⁻¹² M was correlated with a significant reduction in the accumulation of nitrite in the culture medium. The neuroprotective effect of “low” (femtomolar) doses of PACAP in neuron/glia co-cultures was abolished by PD98059, a MAP kinase inhibitor, but the neuroprotective effect of “high” (nanomolar) doses of PACAP was not affected by PD98059 (Li et al., J Mol Neurosci 27:91-106, 2005). However, the neuroprotective effect of nanomolar doses of PACAP was abolished by Rp-cAMP, a protein kinase A inhibitor.

The drawbacks of using peptides for neuroprotection in the brain include their poor transport across the blood-brain barrier and their short half-life in the circulation after systematic administration. However, PACAP38 has been shown to be transported from the blood to the brain via a saturable mechanism (Banks et al., J Pharmacol Exp Ther 267:690-696, 1993). Therefore, PACAP38 was tested as a neuroprotectant in common in vivo preclinical models of heart attack and stroke. Four-vessel occlusion in the rat was used to model the consequences of a heart attack for the brain (transient global forebrain ischemia). Blood flow to the forebrain was interrupted for 15 minutes. Following the 15-minute occlusion, there was a significant reduction in the number of pyramidal cells in the CA1 field of the hippocampus after 7 days in vehicle-infused rats. The reduction in the number of pyramidal cells at day 7 post-occlusion was significantly reversed in the rats continuously infused intravenously with PACAP38 (Uchida et al., Brain Res 736:280-286, 1996). Middle cerebral artery occlusion (MCAO) in the rat was used to model a stroke (transient focal cerebral ischemia). The middle cerebral artery was occluded for 2 hours using the intraluminal filament technique. The continuous intravenous infusion of PACAP38 beginning at 4, 8 or 12 hours after the start of the transient MCAO resulted in a reduction of the infarct volume of approximately 51%, 22% or 12%, respectively, 48 hours after the start of the MCAO (Reglodi et al., Stroke 31:1411-1417, 2000). These observations suggest that small changes in the concentration of PACAP in the brain can alter the vulnerability of nerve cells to injury.

The neuroprotective effects of low concentrations of PACAP in the nervous system are indirect and are probably mediated by at least four distinct mechanisms. (1) PACAP is a potent anti-inflammatory peptide. It has been shown to inhibit the induction of inducible nitric oxide synthase (iNOS) in activated macrophages, to inhibit the production of the pro-inflammatory cytokines TNF-α, IL-6 and IL-12 in activated macrophages, and to stimulate the production of the anti-inflammatory cytokine IL-4 and IL-10 in activated macrophages (Ganea and Delgado, Crit Rev Oral Biol Med 13:229-237, 2002). PACAP probably inhibits inflammation at multiple steps in the inflammatory cascade because it is an endogenous counter-regulator of the inflammatory process. PACAP is also an extraordinarily potent “deactivator” of activated microglial cells (Kong et al., Neuroscience 91:493-500, 1999; Delgado et al., Glia 39:148-161, 2002), which are the resident macrophage-like cells in the nervous system. (2) Femtomolar (10⁻¹⁵ M) concentrations of PACAP increase the levels of the mRNA for activity-dependent neurotrophic factor in murine neuron/glia co-cultures (Nakamachi et al., Peptides 27:1859-1864, 2006). Furthermore, the number of PAC₁ receptors on “reactive” glial cells is increased following injury (Uchida et al., Brain Res 736:280-286, 1996; Nakamachi et al., Acta Neurochir Suppl 118:55-59, 2013). Brenneman et al. (Neuropeptides 36:271-280, 2002) had previously shown that femtomolar concentrations of PACAP stimulate the release of RANTES in astrocyte cultures and that immunoneutralization of RANTES reduces the neuroprotective effect of PACAP in neuron/glia co-cultures. (3) Yang et al. (J Pharmacol Exp Ther 319:595-603, 2006) have shown that femtomolar concentrations of PACAP inhibit microglial NADPH oxidase activity and extracellular superoxide levels in mesencephalic neuron/glia co-cultures. (4) Figiel and Engele (J Neurosci 20:3596-3605, 2000) have reported that PACAP increased the expression of the glutamate transporters GLT-1 and GLAST and increased the activity of the glutamate metabolizing enzyme glutamine synthetase in astrocytes. These effects of PACAP would be expected to decrease glutamatergic neurotransmission. In addition, Mercer et al. (J Neurosci Res 76:205-215, 2004) have reported that infusion of PACAP into the lateral ventricle stimulates the proliferation of neural stem cells in the dentate gyrus of the adult mouse. The extensive studies about the cytoprotective properties of PACAP in the nervous system have provided a solid framework for studying the cytoprotective properties of PACAP in other organs.

Native PACAP has already been administered to healthy human volunteers by investigators in at least seven different laboratories (Warren et al., J Cardiovasc Pharmacol 20:83-87, 1992; Hammond et al., J Endocrinol 137:529-532, 1993; Chiodera et al., Neuroendocrinology 64:242-246, 1996; Birk et al., Regul Pept 140:185-191, 2007; Filipsson et al., J Clin Endocrinol Metab 82:3093-3098, 1997; Doberer et al., Eur J Clin Invest 37:665-672, 2007; Murck et al., Am J Physiol 292:E853-E857, 2007) and to a patient with multiple myeloma under a U.S. Food and Drug Administration (FDA)-approved single-patient protocol (Li et al., Peptides 28:1891-1895, 2007). There were no indications of any serious side-effects during these studies.

PACAP is an extraordinarily potent peptide in vitro. However, the usefulness of PACAP as a drug is limited by its very short half-life in the circulation following systemic administration due to both rapid proteolysis and rapid filtration by the kidney. Therefore, there is a need for PACAP analogs that are resistant to proteolysis and/or have reduced rates of filtration by the kidney in order to optimally treat certain disorders. There is also a need for PACAP analogs that have altered specificities for the three PACAP/VIP receptors in order to increase the therapeutic index for the treatment of certain disorders such as acute neurological diseases, type II diabetes and keratoconjunctivitis sicca (dry eye syndrome).

Citation or discussion of a reference herein shall not be construed as an admission that such reference is prior art to the present invention.

### SUMMARY OF THE INVENTION

The inventors have made peptide analogs of native human PACAP38 that are agonists at one or more PACAP/VIP receptors, and that have significant biological activity in preclinical in vitro and in vivo models for several major medical disorders. The PACAP analogs of this invention can be synthesized by the standard methods of peptide chemistry.

In a first aspect, the invention features PACAP analogs that can be used for the prophylactic/therapeutic and diagnostic purposes described in more detail below can be defined by a general formula (I),

(SEQ ID NO: 15)
A¹-A²-A³-A⁴-A⁵-A⁶-A⁷-A⁸-A⁹-A¹⁰-A¹¹-A¹²-A¹³-A¹⁴-
A¹⁵-A¹⁶-A¹⁷-A¹⁸-A¹⁹-A²⁰-A²¹-A²²-A²³-A²⁴-A²⁵-A²⁶-
A²⁷-A²⁸-A³⁰-A³¹-A³²-A³³-A³⁴-A³⁵-A³⁶-A³⁷-A³⁸-R¹

or a pharmaceutically acceptable salt thereof, where

### A¹ is Iaa, Iac, Ica, or Paa;

A² is Ser, D-Ser, hSer, N-Me-Ser, Thr, D-Thr, D-Tyr, Ala, D-Ala, Ile, D-Ile, Pro, Hyp, Abu, Aib, Acb, Ach, Acpe, or Acpr;

### A³ is Asp, D-Asp, Glu, D-Glu, Asn, D-Asn, or N-Me-Asp;

### A⁴ is Gly, Sar, Ala, D-Ala, β-Ala, Gaba, Abu, Aib, Acb, Ach, Acpe, or Acpr;

### A⁵ is Ile, Leu, Nle, Val, Nva, Aib, Acb, Ach, Acpe, or Acpr;

### A⁶ is Phe, Tyr, Pse, Trp, Cha, Bip, Pal, or Nal;

A⁷ is Thr, Ser, hSer, Val, Nva, Ala, or Aib;

### A⁸ is Asp, Asn, or Glu;

A⁹ is Ser, hSer, Thr, Asn, Asp, Ala, Abu, Aib, Acb, Ach, Acpe, or Acpr;

### A¹⁰ is Tyr, Phe, Pse, Dopa, Cha, Pal, Nal, Trp, Ala, or Aib;

A¹¹ is Ser, hSer, Thr, Ala, Abu, Aib, Acb, Ach, Acpe, or Acpr;

### A¹² is Arg, Lys, Dab, Dap, or Orn;

### A¹³ is Tyr, Phe, Pse, Dopa, Cha, Pal, Nal, or Trp;

### A¹⁴ is Arg, Lys, Dab, Dap, Orn, Asn, or Gln;

### A¹⁵ is Lys, Ala, Dab, Dap, Orn, Abu, Aib, Acb, Ach, Acpe, Arg, or Acpr;

### A¹⁶ is Gln, Glu, Asn, Asp, Ala, Aib, Acb, Ach, Acpe, or Acpr;

### A¹⁷ is Met, Nle, Nva, Leu, Ile, Ala, Abu, Aib, Acb, Ach, Acpe, or Acpr;

### A¹⁸ is Ala, Abu, Aib, Acb, Ach, Acpe, or Acpr;

### A¹⁹ is Val, Nva, Ser, Leu, Thr, Ala, Aib, Acb, Ach, Acpe, or Acpr;

### A²⁰ is Lys, Ala, Dab, Dap, Orn, Abu, Aib, Acb, Ach, Acpe, Arg, or Acpr;

### A²¹ is Lys, Ala, Dab, Dap, Orn, Abu, Aib, Acb, Ach, Acpe, Arg, or Acpr;

### A²² is Tyr, Phe, Pse, Dopa, Cha, Pal, Nal, Trp, Ala, Abu, Aib, Acb, Ach, Acpe, or Acpr;

### A²³ is Leu, Nle, Ile, Val, Nva, Abu, Aib, Acb, Ach, Acpe, or Acpr;

### A²⁴ is Ala, Asn, Abu, Aib, Acb, Ach, Acpe, or Acpr;

A²⁵ is Ala, Val, Leu, Met, Nle, Ile, Ser, hSer, Thr, Abu, Aib, Acb, Ach, Acpe, Acpr, or is omitted;
A²⁶ is Val, Nva, Leu, Met, Nle, Ile, Ala, Abu, Aib, Acb, Ach, Acpe, Acpr, or is omitted;
A²⁷ is Leu, D-Leu, Met, D-Met, Nle, Ile, D-He, Val, D-Val, Gaba, Ala, D-Ala, Abu, Aib, Acb, Ach, Acpe, Acpr, or is omitted;
A²⁸ is Gly, Sar, Ala, D-Ala, β-Ala, Gaba, Asn, D-Asn, Gln, D-Gln, Asp, D-Asp, Abu, Aib, Acb, Ach, Acpe, Acpr, or is omitted;
A²⁹ is Lys, D-Lys, Arg, D-Arg, Dab, D-Dab, Dap, D-Dap, Orn, D-Orn, or is omitted;
A³⁰ is Arg, D-Arg, Lys, D-Lys, Dab, D-Dab, Dap, D-Dap, Orn, D-Orn, or is omitted;
A³¹ is Tyr, D-Tyr, Phe, D-Phe, Pse, D-Pse, Dopa, D-Dopa, Trp, D-Trp, Cha, Pal, Nal, or is omitted;
A³² is Lys, D-Lys, Arg, D-Arg, Dab, D-Dab, Dap, D-Dap, Orn, D-Orn, or is omitted;
A³³ is Gln, D-Gln, Glu, D-Glu, Asn, D-Asn, Asp; D-Asp, Abu, Aib, Acb, Ach, Acpe, Acpr, or is omitted;
A³⁴ is Arg, D-Arg, Lys, D-Lys, Dab, D-Dab, Dap, D-Dap, Orn, D-Orn, or is omitted;
A³⁵ is Val, D-Val, Nva, Ser, D-Ser, Thr; D-Thr, Abu, Aib, Acb, Ach, Acpe, Acpr, or is omitted;
A³⁶ is Lys, D-Lys, Arg, D-Arg, Dab, D-Dab, Dap, D-Dap, Orn, D-Orn, or is omitted;
A³⁷ is Asn, D-Asn, Gln, D-Gln, Asp, D-Asp, Ala, D-Ala, Aib, Acb, Ach, Acpe, Acpr, or is omitted;
A³⁸ is Lys, D-Lys, Arg, D-Arg, Dab, D-Dab, Dap, D-Dap, Orn, D-Orn, or is omitted;
R¹ is independently selected from a group of OH, NH₂, (C₁-C₁₈)alkoxyl, and NH(C₁-C₁₈)alkyl.

In some embodiments, the compound is selected from the following PACAP analogs, or pharmaceutically acceptable salts thereof:

(SEQ ID NO: 4)
Iac Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 5)
Iaa Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 6)
Iaa Ser Asp Gly Ile Phe Thr Asp Ser Tyr Ser Arg
Tyr Arg Lys Ala Ala Ala Val Lys Lys Tyr Leu Ala
Ala Val Leu Gly Lys Arg Tyr Lys Gln Arg Val Lys
Asn D-Lys-NH₂;
(SEQ ID NO: 7)
Iaa D-Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 8)
Iac Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂,
(SEQ ID NO: 9)
Iaa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 10)
Paa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 11)
Iaa D-Ser Asp Gly Ile Phe Thr Asp Ser Tyr Ser
Arg Tyr Arg Lys Gln Met Ala Val Lys Lys Ala Leu
Ala Ala Val Leu Gly Lys Arg Tyr Lys Gln Arg Val
Lys Asn Lys-NH₂;
(SEQ ID NO: 12)
Iaa D-Tyr Asp Gly Ile Phe Thr Asp Ser Tyr Ser
Arg Tyr Arg Lys Gln Met Ala Val Lys Lys Ala Leu
Ala Ala Val Leu Gly Lys Arg Tyr Lys Gln Arg Val
Lys Asn Lys-NH₂;
(SEQ ID NO: 13)
Iaa D-Ala Asp Gly Ile Phe Thr Asp Ser Tyr Ser
Arg Tyr Arg Lys Gln Met Ala Val Lys Lys Ala Leu
Ala Ala Val Leu Gly Lys Arg Tyr Lys Gln Arg Val
Lys Asn Lys-NH₂;
and
(SEQ ID NO: 14)
Iac D-Ser Asp Gly Ile Phe Thr Asp Ser Tyr Ser
Arg Tyr Arg Lys Gln Met Ala Val Lys Lys Ala Leu
Ala Ala Val Leu Gly Lys Arg Tyr Lys Gln Arg Val
Lys Asn Lys-NH₂.

In a second aspect, the invention features a PACAP analog having at least 90% sequence identity to a sequence selected from any one of SEQ ID NOs: 4-78 (e.g., any one of SEQ ID NOs: 4-14), in which the analog includes an imidazole-4-acetic acid (Iaa), an imidazole-4-acrylic acid (Iac), an imidazole-4-carboxylic acid (Ica), or 3-pyridylacetic acid (Paa) at position 1. In some embodiments, the amino acid residue at position 2 of the PACAP analog is selected from Ser, D-Ser, hSer, N-Me-Ser, Thr, D-Thr, D-Tyr, Ala, D-Ala, Ile, D-He, Pro, Hyp, Abu, Aib, Acb, Ach, Acpe, or Acpr and/or the amino acid residue at position 16 of the PACAP analog is selected from Gln, Glu, Asn, Asp, Ala, Aib, Acb, Ach, Acpe, and Acpr and/or the amino acid residue at position 17 of the PACAP analog is selected from Met, Nle, Leu, Ile, Ala, Abu, Aib, Acb, Ach, Acpe, and Acpr and/or the amino acid residue at position 22 of the PACAP analog is selected from Tyr, Phe, Pse, Dopa, Cha, Pal, Nal, Trp, Ala, Abu, Aib, Acb, Ach, Acpe, and Acpr and/or the amino acid residue at position 38 of the PACAP analog is selected from Lys, D-Lys, Arg, D-Arg, Dab, D-Dab, Dap, D-Dap, Orn, and D-Orn, or is omitted. In an embodiment, the PACAP analog has one or more (e.g., 1, 2, 3, or 4) conservative amino acid substitutions (e.g., at one or more of positions 2, 16, 17, 22, and 38).

In some embodiments, the PACAP analog has at least 95% (e.g., at least 99%) sequence identity to a sequence selected from any one of SEQ ID NOs: 4-78 (e.g., any one of SEQ ID NOs: 4-14).

In other embodiments, the PACAP analog is present in a composition having a pharmaceutically acceptable carrier.

In some embodiments, the PACAP analog is conjugated to one or more radionuclides or small molecules. In particular, the radionuclide is ¹¹C, ¹³N, ¹⁵O, ¹⁸F, ⁵²Fe, ⁵⁵Co, ⁶¹Cu, ⁶²Cu, ⁶⁴Cu, ⁶⁷Cu, ⁶⁷Ga, ⁶⁸Ga, ⁶²Zn, ⁶³Zn, ⁷⁰As, ⁷¹As, ⁷⁴As, ⁷⁶Br, ⁷⁹Br, ⁸²Rb, ⁸⁶Y, ⁸⁹Zr, ¹¹⁰In, ¹¹¹In, ¹²⁰I, ¹²³I, ¹²⁴I, ¹²⁵I, ¹³¹I, ¹²²Xe, ¹⁷⁵Lu, ¹⁵⁴Gd, ¹⁵⁵Gd, ¹⁵⁶Gd, ¹⁵⁷Gd, ¹⁵⁸Gd, ^{94m}Tc, ⁹⁴Tc, or ^{99m}Tc. In particular, the small molecule is a therapeutic or anticancer agent. Specifically, the therapeutic or anticancer agent is cisplatin, carboplatin, oxaliplatin, bleomycin, mitomycin C, calicheamicins, maytansinoids, auristatins, esperamicins, geldanamycin, doxorubicin, idarubicin, daunorubicin, epirubicin, busulfan, carmustine (BCNU), lomustine (CCNU), semustine, fotemustine, bendamustine, nimustine, thalidomide, lenalidomide, methotrexate, azathioprine, 6-mercaptopurine, fludarabine, 5-azacytidine, pentostatin (2′-deoxycoformycin), cytarabine (cytosine arabinoside), gemcitabine, 5-fluorouracil, hydroxyurea, elesclomol, etoposide, teniposide, amsacrine, mitoxantrone, camptothecin, topotecan, irinotecan, chlorambucil, cyclophosphamide, ifosfamide, melphalan, bortezomib, vincristine, vinblastine, vinorelbine, paclitaxel, docetaxel, iobitridol, iodipamide, iodixanol, iohexol, iomeprol, iopamidol, iopentol, iopromide, iotrolan, ioversol, ioxilan, iothalamate, ioxithalamate, ioxaglate, metrizamide, acetrizoate, metrizoate, diatrizoate, cyclosporine A, tacrolimus (FK506), sirolimus (rapamycin), everolimus, temsirolimus, zotarolimus, or biolimus.

A third aspect of the invention features a method for treating, managing, or preventing a disease selected from an age-related neurodegenerative disease, a central nervous system disorder, Huntington's disease or other CAG codon repeat expansion disease, a retinal disease, an autoimmune disease, graft-versus-host disease, keratoconjunctivitis sicca caused by aging, autoimmune diseases or keratorefractive surgery, type II diabetes, sepsis caused by a bacteria and/or a virus, an acute or chronic cardiovascular disease, an acute or chronic renal disease, a genetic disorder caused by a premature in-frame stop codon, an acute or chronic pulmonary disease, systemic hypertension, a hematological cancer, a granuloma, an eating disorder, an acute or chronic liver disease, osteoporosis, pre-eclampsia, cell and solid organ transplantation, a cognitive disorder, acquired immunodeficiency syndrome (AIDS) dementia complex, and aging of the central nervous system by administering to a subject in need thereof an effective amount of one or more of the PACAP analogs of the first or second aspect of the invention or a pharmaceutically acceptable salt thereof.

In some embodiments, the age-related neurodegenerative disease is selected from Alzheimer's disease, Parkinson's disease, and amyotrophic lateral sclerosis. The central nervous system disorder is caused by stroke, heart attack or blunt force trauma, wherein preferably the blunt force trauma is a concussion or spinal cord trauma. The retinal disease is ischemia/reperfusion injury, non-infectious uveitis, diabetic retinopathy, macular degeneration, or glaucoma. The autoimmune disease is rheumatoid arthritis, Crohn's disease, ulcerative colitis, scleroderma, Sjögren's disease, idiopathic membranous nephropathy, Goodpasture's disease, autoimmune hepatitis, myasthenia gravis, multiple sclerosis, Guillain-Barré syndrome, type I diabetes, Hashimoto's thyroiditis, Graves' disease, pemphigus vulgaris, or systemic lupus erythematosus. The sepsis is caused by a bacteria or a virus. The acute or chronic cardiovascular disease is myocardial infarction, atherosclerosis, restenosis, or a drug-induced cardiomyopathy. The acute or chronic renal disease is ischemia/reperfusion injury, nephritis, or drug-induced nephrotoxicity. The acute or chronic pulmonary disease is asthma, chronic obstructive pulmonary disease, cystic fibrosis, or pulmonary arterial hypertension. The hematological cancer is a lymphoid or myeloid hematopoietic cancer, in which preferably the lymphoid or myeloid hematopoietic cancer is a leukemia, a lymphoma, or a plasma cell dyscrasia. The acute or chronic liver disease is ischemia/reperfusion injury, hepatitis, or fatty liver. The genetic disorder caused by a premature in-frame stop codon is cystic fibrosis, Duchenne muscular dystrophy, Krabbe's disease (globoid cell leukodystrophy), Hurler's syndrome, retinitis pigmentosa, ataxia telangiectasia, nephropathic cystinosis, or polycystic kidney disease. The keratoconjunctivitis sicca is caused by aging, an autoimmune disease or keratorefractive surgery.

In other embodiments, the subject has an injury to one or more major organs of the body due to treatment with a therapeutic or anticancer agent other than the PACAP analog, trauma, or acute or chronic disease. In particular, the one or more major organs of body is selected from nervous system, brain, spinal cord, heart, lung, kidneys, liver, pancreas, gall bladder, gastrointestinal tract, adrenal gland, thymus, spleen, lymph nodes, breast, ovary, testes, cornea, and prostate, preferably in which the one or more major organs of the body is selected from nervous system, heart, lung, kidneys, liver, cornea, and gastrointestinal tract.

In some embodiments, the PACAP analogs of the invention, or pharmaceutically acceptable salts thereof, bind to one or more of the PACAP/VIP receptors and/or reduce one or more injuries to one or more major organs of the body of the subject due to treatment with a therapeutic or anticancer agent other than the PACAP analog, trauma, or acute or chronic disease.

In some embodiments, the PACAP analog is selected from one or more of the following:

(SEQ ID NO: 4)
Iac Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 5)
Iaa Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 6)
Iaa Ser Asp Gly Ile Phe Thr Asp Ser Tyr Ser Arg
Tyr Arg Lys Ala Ala Ala Val Lys Lys Tyr Leu Ala
Ala Val Leu Gly Lys Arg Tyr Lys Gln Arg Val Lys
Asn D-Lys-NH₂;
(SEQ ID NO: 7)
Iaa D-Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 8)
Iac Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 9)
Iaa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 10)
Paa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 11)
Iaa D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 12)
Iaa D-Tyr Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 13)
Iaa D-Ala Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 14)
Iac D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂.

In other embodiments, the PACAP analog has at least 90% sequence identity to a sequence selected from any one of SEQ ID NOs: 4-78 (e.g., any one of SEQ ID NOs: 4-14), in which the analog includes an Iaa, an Iac, an Ica, or a Paa at position 1.

In some embodiments, the disease is a hematological cancer.

In other embodiments, the disease is an autoimmune disease.

In yet other embodiments, the subject is resistant to treatment with a glucocorticoid. In particular, the glucocorticoid is dexamethasone, prednisolone, methylprednisolone, or prednisone.

In still other embodiments, the hematological cancer is multiple myeloma.

In some embodiments, the PACAP analog to be administered has the sequence of any one of SEQ ID NOs: 4-78 (e.g., any one of SEQ ID NOs: 4-14). In particular, the PACAP analog has the sequence of SEQ ID NO: 12.

In some embodiments, the administration of the PACAP analog in the subject replaces the corticosteroid (prednisone or dexamethasone) using the COP (cyclophosphamide, Oncovin [vincristine] and prednisone) or VAD (vincristine, Adriamycin [doxorubicin] and dexamethasone) regimen.

In some embodiments, the PACAP analog or a pharmaceutically acceptable salt thereof is linked to a polyethylene glycol polymer with a molecular weight from about 4 kilodaltons to about 40 kilodaltons.

In other embodiments, the PACAP analog is the unamidated (free acid) form flanked by amino-acid consensus sequences for one or more proteolytic enzymes.

In yet other embodiments, the PACAP analog is a peptidomimetic analog.

In some embodiments, the PACAP analog is administered at a dosage that produces a concentration of 10⁻¹⁴ M to 10⁻⁶ M in the blood of the subject.

In other embodiments, the PACAP analog is administered by intravenous infusion at a rate of about 1 pmol/kg body weight/hour to about 20 pmol/kg body weight/hour.

In other embodiments, the administration by intravenous infusion is for about 1-12 hours. In some embodiments of the third aspect of the invention, the injuries to one or more major organs of the body are due to treatment with one or more of cisplatin, carboplatin, oxaliplatin, bleomycin, mitomycin C, calicheamicins, maytansinoids, auristatins, esperamicins, geldanamycin, doxorubicin, idarubicin, daunorubicin, epirubicin, busulfan, carmustine (BCNU), lomustine (CCNU), semustine, fotemustine, bendamustine, nimustine, thalidomide, lenalidomide, methotrexate, azathioprine, 6-mercaptopurine, fludarabine, 5-azacytidine, pentostatin (2′-deoxycoformycin), cytarabine (cytosine arabinoside), gemcitabine, 5-fluorouracil, hydroxyurea, elesclomol, etoposide, teniposide, amsacrine, mitoxantrone, camptothecin, topotecan, irinotecan, chlorambucil, cyclophosphamide, ifosfamide, melphalan, bortezomib, vincristine, vinblastine, vinorelbine, paclitaxel, docetaxel, G418, gentamicin, streptomycin, kanamycin, tobramycin, amikacin, arbekacin, dibekacin, neomycin, netilmicin, paromomycin, bekanamycin, hygromycin B, apramycin, sisomicin, isepamicin, astromicin, verdamicin, amphotericin B, rifampicin, pentamidine, iobitridol, iodipamide, iodixanol, iohexol, iomeprol, iopamidol, iopentol, iopromide, iotrolan, ioversol, ioxilan, iothalamate, ioxithalamate, ioxaglate, metrizamide, acetrizoate, metrizoate, diatrizoate, cyclosporine A, tacrolimus (FK506), sirolimus (rapamycin), everolimus, temsirolimus, zotarolimus, or biolimus.

In other embodiments, the injury is to a kidney of the subject due to treatment with one or more of cisplatin, carboplatin, carmustine, lomustine, semustine, fotemustine, ifosfamide, methotrexate, pentostatin, 5-azacytidine, doxorubicin, daunorubicin, hydroxyurea, mitomycin C, G418, gentamicin, streptomycin, kanamycin, tobramycin, amikacin, arbekacin, dibekacin, neomycin, netilmicin, paromomycin, bekanamycin, hygromycin B, apramycin, sisomicin, isepamicin, astromicin, verdamicin, amphotericin B, rifampicin, pentamidine, iobitridol, iodipamide, iodixanol, iohexol, iomeprol, iopamidol, iopentol, iopromide, iotrolan, ioversol, ioxilan, iothalamate, ioxithalamate, ioxaglate, metrizamide, acetrizoate, metrizoate, diatrizoate, mitoxantrone, cyclosporine A, tacrolimus (FK506), sirolimus (rapamycin), everolimus, temsirolimus, zotarolimus, or biolimus.

In yet other embodiments, the PACAP analog is injected intraperitoneally one or more times per day.

In still other embodiments, the PACAP analog is injected subcutaneously one or more times per week.

In other embodiments, the PACAP analog is injected intramuscularly one or more times per week.

In other embodiments, the PACAP analog is administered intranasally one or more times per day.

In other embodiments, the PACAP analog is administered as an aerosol one or more times per day.

In yet other embodiments, the PACAP analog is administered orally in a time-dependent or pH-dependent formulation one or more times per day.

In still other embodiments, the PACAP analog is administered as a controlled release or a sustained release formulation.

In some embodiments, the PACAP analog is administered after encapsulation in liposomes or microparticles.

In some embodiments, the PACAP analog is administered transcutaneously after encapsulation in dendrimers.

In other embodiments, the PACAP analog is used to coat a metallic or a biodegradable stent.

In yet other embodiments, the PACAP analog is administered in combination with one or more other cytoprotective adjuvants. In particular, the cytoprotective adjuvant is amifostine, dexrazoxane, mesna, palifermin, apocynin, erythropoietin, N-acetylcysteine, or N-acetylcysteine amide.

In some embodiments, the injuries to one or more major organs of the body are due to treatment with an unconjugated therapeutic or anticancer agent, a therapeutic or anticancer agent conjugated to a monoclonal antibody or a bioactive peptide, or an unconjugated bioactive peptide.

In some embodiments, the PACAP analog or a pharmaceutically acceptable salt thereof, is conjugated to a therapeutic or anticancer agent.

In other embodiments, the PACAP analog has an additive anticancer effect with one or more other anticancer agents.

In some embodiments, the subject is being treated with one or more anticancer agents for a hematopoietic cancer.

In some embodiments, the subject is being treated with one or more therapeutic or anticancer agents for a myeloproliferative disorder.

In some embodiments, the subject is being treated with one or more therapeutic or anticancer agents for multiple myeloma.

In all aspects of the invention, the subject is a mammal (e.g., a human).

A fourth aspect of the invention features a method for the localization, diagnosis, or treatment of a disseminated cancer and metastatic tumor in a subject by administering an effective amount of a conjugate including one or more of the PACAP analogs of the polypeptides of the preceding embodiments or a pharmaceutically acceptable salt thereof coupled to one or more radionuclides.

In some embodiments, the one or more of the PACAP analogs bind to one or more of PACAP/VIP receptor on the surface of one or more cells of the disseminated cancer or metastatic tumor.

In some embodiments of the method, the PACAP analog is selected from one or more of the following:

(SEQ ID NO: 4)
Iac Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 5)
Iaa Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 6)
Iaa Ser Asp Gly Ile Phe Thr Asp Ser Tyr Ser Arg
Tyr Arg Lys Ala Ala Ala Val Lys Lys Tyr Leu Ala
Ala Val Leu Gly Lys Arg Tyr Lys Gln Arg Val Lys
Asn D-Lys-NH₂;
(SEQ ID NO: 7)
Iaa D-Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 8)
Iac Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 9)
Iaa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 10)
Paa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 11)
Iaa D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 12)
Iaa D-Tyr Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 13)
Iaa D-Ala Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 14)
Iac D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂.

In other embodiments, the PACAP analog has at least 90% sequence identity to a sequence selected from any one of SEQ ID NOs: 4-78 (e.g., any one of SEQ ID NOs: 4-14), in which the analog includes an Iaa, an Iac, an Ica, or a Paa at position 1.

In some embodiments of the method, the disseminated cancer and metastatic tumor is a hematological cancer. In particular, the hematological cancer is a leukemia, lymphoma, or plasma cell dyscrasia.

In some embodiments, the PACAP analog binds to a target cell that is a component of a granuloma caused by one or more infectious agents or an autoimmune disease.

In other embodiments, the subject is being treated with one or more of the conjugates for lymphoid or myeloid cancer.

In other embodiments, the subject is being treated with one or more of the conjugates for multiple myeloma.

In embodiment, the subject is a mammal (e.g., a human).

A fifth aspect of the invention features a method of producing a conjugate by coupling one or more radionuclides or small molecules to one or more of the PACAP analogs of the invention. In some embodiments, the PACAP analog is selected from one or more of the following, or a pharmaceutically acceptable salt thereof:

(SEQ ID NO: 4)
Iac Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 5)
Iaa Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 6)
Iaa Ser Asp Gly Ile Phe Thr Asp Ser Tyr Ser Arg
Tyr Arg Lys Ala Ala Ala Val Lys Lys Tyr Leu Ala
Ala Val Leu Gly Lys Arg Tyr Lys Gln Arg Val Lys
Asn D-Lys-NH₂;
(SEQ ID NO: 7)
Iaa D-Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 8)
Iac Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 9)
Iaa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 10)
Paa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 11)
Iaa D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 12)
Iaa D-Tyr Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 13)
Iaa D-Ala Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 14)
Iac D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂.

In other embodiments, the PACAP analog has at least 90% sequence identity to a sequence selected from any one of SEQ ID NOs: 4-78 (e.g., any one of SEQ ID NOs: 4-14), in which the analog includes an Iaa, an Iac, an Ica, or a Paa at position 1.

In some embodiments, the radionuclide is selected from ¹¹C, ¹³N, ¹⁵O, ¹⁸F, ⁵²Fe, ⁵⁵Co, ⁶¹Cu, ⁶²Cu, ⁶⁴Cu, ⁶⁷Cu, ⁶⁷Ga, ⁶⁸Ga, ⁶²Zn, ⁶³Zn, ⁷⁰As, ⁷¹As, ⁷⁴As, ⁷⁶Br, ⁷⁹Br, ⁸²Rb, ⁸⁶Y, ⁸⁹Zr, ¹¹⁰In, ¹¹¹In, ¹²⁰I, ¹²³I, ¹²⁴I, ¹²⁵I, ¹³¹I, ¹²²Xe, ¹⁷⁵Lu, ¹⁵⁴Gd, ¹⁵⁵Gd, ¹⁵⁶Gd, ¹⁵⁷Gd, ¹⁵⁸Gd, ^{94m}Tc, ⁹⁴Tc, and ^{99m}Tc.

In some embodiments, the small molecule is a therapeutic or anticancer agent. In particular, the therapeutic or anticancer agent is cisplatin, carboplatin, oxaliplatin, bleomycin, mitomycin C, calicheamicins, maytansinoids, auristatins, esperamicins, geldanamycin, doxorubicin, idarubicin, daunorubicin, epirubicin, busulfan, carmustine (BCNU), lomustine (CCNU), semustine, fotemustine, bendamustine, nimustine, thalidomide, lenalidomide, methotrexate, azathioprine, 6-mercaptopurine, fludarabine, 5-azacytidine, pentostatin (2′-deoxycoformycin), cytarabine (cytosine arabinoside), gemcitabine, 5-fluorouracil, hydroxyurea, elesclomol, etoposide, teniposide, amsacrine, mitoxantrone, camptothecin, topotecan, irinotecan, chlorambucil, cyclophosphamide, ifosfamide, melphalan, bortezomib, vincristine, vinblastine, vinorelbine, paclitaxel, docetaxel, iobitridol, iodipamide, iodixanol, iohexol, iomeprol, iopamidol, iopentol, iopromide, iotrolan, ioversol, ioxilan, iothalamate, ioxithalamate, ioxaglate, metrizamide, acetrizoate, metrizoate, diatrizoate, cyclosporine A, tacrolimus (FK506), sirolimus (rapamycin), everolimus, temsirolimus, zotarolimus, or biolimus.

A sixth aspect of the invention features a method for targeted delivery of a therapeutic or anticancer agent to a specific cell or tissue of a subject by administering to the subject an effective amount of a conjugate including one or more PACAP analogs of polypeptides of the preceding embodiments, or a pharmaceutically acceptable salt thereof, coupled to one or more small molecules. In an embodiment, the small molecule is a therapeutic or anticancer agent selected from cisplatin, carboplatin, oxaliplatin, bleomycin, mitomycin C, calicheamicins, maytansinoids, auristatins, esperamicins, geldanamycin, doxorubicin, idarubicin, daunorubicin, epirubicin, busulfan, carmustine (BCNU), lomustine (CCNU), semustine, fotemustine, bendamustine, nimustine, thalidomide, lenalidomide, methotrexate, azathioprine, 6-mercaptopurine, fludarabine, 5-azacytidine, pentostatin (2′-deoxycoformycin), cytarabine (cytosine arabinoside), gemcitabine, 5-fluorouracil, hydroxyurea, elesclomol, etoposide, teniposide, amsacrine, mitoxantrone, camptothecin, topotecan, irinotecan, chlorambucil, cyclophosphamide, ifosfamide, melphalan, bortezomib, vincristine, vinblastine, vinorelbine, paclitaxel, docetaxel, iobitridol, iodipamide, iodixanol, iohexol, iomeprol, iopamidol, iopentol, iopromide, iotrolan, ioversol, ioxilan, iothalamate, ioxithalamate, ioxaglate, metrizamide, acetrizoate, metrizoate, diatrizoate, cyclosporine A, tacrolimus (FK506), sirolimus (rapamycin), everolimus, temsirolimus, zotarolimus, and biolimus.

In some embodiments, the one or more of the PACAP analogs bind to one or more PACAP/VIP receptors on the surface of the cell or tissue and the conjugate enters the interior of the cell or tissue by receptor-mediated endocytosis.

In some embodiments, the PACAP analog is selected from one or more of the following, or a pharmaceutically acceptable salt thereof:

(SEQ ID NO: 4)
Iac Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 5)
Iaa Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 6)
Iaa Ser Asp Gly Ile Phe Thr Asp Ser Tyr Ser Arg
Tyr Arg Lys Ala Ala Ala Val Lys Lys Tyr Leu Ala
Ala Val Leu Gly Lys Arg Tyr Lys Gln Arg Val Lys
Asn D-Lys-NH₂;
(SEQ ID NO: 7)
Iaa D-Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 8)
Iac Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 9)
Iaa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 10)
Paa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 11)
Iaa D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 12)
Iaa D-Tyr Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 13)
Iaa D-Ala Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 14)
Iac D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂.

In other embodiments, the PACAP analog has at least 90% sequence identity to a sequence selected from any one of SEQ ID NOs: 4-78 (e.g., any one of SEQ ID NOs: 4-14), in which the analog includes an Iaa, an Iac, an Ica, or a Paa at position 1.

In some embodiments, the subject has a disease. In particular, the disease is selected from an age-related neurodegenerative disease, a central nervous system disorder, Huntington's disease or other CAG codon repeat expansion disease, a retinal disease, an autoimmune disease, graft-versus-host disease, keratoconjunctivitis sicca caused by aging, autoimmune diseases or keratorefractive surgery, type II diabetes, sepsis caused by a bacteria and/or a virus, an acute or chronic cardiovascular disease, an acute or chronic renal diseases, a genetic disorder caused by a premature in-frame stop codon, an acute or chronic pulmonary disease, systemic hypertension, a hematological cancer, an eating disorder, an acute or chronic liver disease, osteoporosis, pre-eclampsia, cell and solid organ transplantation, a cognitive disorder, acquired immunodeficiency syndrome (AIDS) dementia complex, and aging of the central nervous system.

In some embodiments, the age-related neurodegenerative disease is selected from Alzheimer's disease, Parkinson's disease, and amyotrophic lateral sclerosis. The central nervous system disorder is caused by stroke, heart attack or blunt force trauma, wherein preferably the blunt force trauma is a concussion or spinal cord trauma. The retinal disease is ischemia/reperfusion injury, non-infectious uveitis, diabetic retinopathy, macular degeneration, or glaucoma. The autoimmune disease is rheumatoid arthritis, Crohn's disease, ulcerative colitis, scleroderma, Sjögren's disease, idiopathic membranous nephropathy, Goodpasture's disease, autoimmune hepatitis, myasthenia gravis, multiple sclerosis, Guillain-Barré syndrome, type I diabetes, Hashimoto's thyroiditis, Graves' disease, pemphigus vulgaris, or systemic lupus erythematosus. The sepsis is caused by a bacteria or a virus. The acute or chronic cardiovascular disease is myocardial infarction, atherosclerosis, or restenosis, restenosis, or a drug-induced cardiomyopathy. The acute or chronic renal disease is ischemia/reperfusion injury, nephritis, or drug-induced nephrotoxicity. The acute or chronic pulmonary disease is asthma, chronic obstructive pulmonary disease, cystic fibrosis, or pulmonary arterial hypertension. The hematological cancer is a lymphoid or myeloid hematopoietic cancer, wherein preferably the lymphoid or myeloid hematopoietic cancer is a leukemia, a lymphoma, or a plasma cell dyscrasia. The acute or chronic liver disease is ischemia/reperfusion injury, hepatitis, or fatty liver. The genetic disorder caused by a premature in-frame stop codon is cystic fibrosis, Duchenne muscular dystrophy, Krabbe's disease (globoid cell leukodystrophy), Hurler's syndrome, retinitis pigmentosa, ataxia telangiectasia, nephropathic cystinosis, or polycystic kidney disease. The keratoconjunctivitis sicca is caused by aging, an autoimmune disease, corneal transplantation, or keratorefractive surgery.

In some embodiments, the disease causes injury to one or more major organs of the body of the subject due to treatment with a therapeutic or anticancer agent other than the PACAP analog, trauma, or acute or chronic disease.

In some embodiments, the conjugate, or a pharmaceutically acceptable salt thereof, binds to one or more of the PACAP/VIP receptors and/or reduces one or more injuries to one or more major organs of the body of the subject due to treatment with a therapeutic or anticancer agent other than the PACAP analog, trauma, or acute or chronic disease.

In some embodiments, the one or more major organs of the body are selected from nervous system, brain, spinal cord, heart, lung, kidneys, liver, pancreas, gall bladder, gastrointestinal tract, adrenal gland, thymus, spleen, lymph nodes, breast, ovary, testes, cornea, and prostate, preferably in which one or more major organs of the body are selected from nervous system, heart, lung, kidneys, liver, cornea, and gastrointestinal tract.

In other embodiments, the disease is cancer or an autoimmune disease.

In some embodiments, the small molecule is anti-inflammatory agent and the subject is being treated for rheumatoid arthritis.

In other embodiments, the small molecule is a anticancer agent and the subject is being treated for multiple myeloma.

In an embodiment, the subject is a mammal (e.g., a human).

A seventh aspect of the invention features a method for detecting a granuloma in subject by administering to the subject an effective amount of a PACAP analog of the invention or a pharmaceutically acceptable salt thereof, conjugated to a radionuclide. In some embodiments, the radionuclide is ¹¹C, ¹³N, ¹⁵O, ¹⁸F, ⁵²Fe, ⁵⁵Co, ⁶¹Cu, ⁶²Cu, ⁶⁴Cu, ⁶⁷Cu, ⁶⁷Ga, ⁶⁸Ga, ⁶²Zn, ⁶³Zn, ⁷⁰As, ⁷¹As, ⁷⁴As, ⁷⁶Br, ⁷⁹Br, ⁸²Rb, ⁸⁶Y, ⁸⁹Zr, ¹¹⁰In, ¹¹¹In, ¹²⁰I, ¹²³I, ¹²⁴I, ¹²⁵I, ¹³¹I, ¹²²Xe, ¹⁷⁵Lu, ¹⁵⁴Gd, ¹⁵⁵Gd, ¹⁵⁶Gd, ¹⁵⁷Gd, ¹⁵⁸Gd, ^{94m}Tc, ⁹⁴Tc, or ^{99m}Tc.

In other embodiments, the subject has an infectious or autoimmune disease.

In some embodiments, the PACAP analog is capable of binding to one or more of PACAP/VIP receptors on the surface of target cells.

In other embodiments, the subject is being treated for tuberculosis.

In other embodiments, the subject is being treated with one or more of the conjugates including an imaging agent for tuberculosis.

In yet other embodiments, the subject is being treated with ^{99m}Tc-isonicotinylhydrazine (INH).

In still other embodiments, the subject is being treated for Crohn's disease. In particular, the subject is being treated with one or more of the conjugates including an imaging agent for Crohn's disease.

In some embodiments, the subject is being treated with a primary therapeutic selected from one or more of cisplatin, carboplatin, oxaliplatin, bleomycin, mitomycin C, calicheamicins, maytansinoids, auristatins, esperamicins, geldanamycin, doxorubicin, idarubicin, daunorubicin, epirubicin, busulfan, carmustine (BCNU), lomustine (CCNU), semustine, fotemustine, bendamustine, nimustine, thalidomide, lenalidomide, methotrexate, azathioprine, 6-mercaptopurine, fludarabine, 5-azacytidine, pentostatin (2′-deoxycoformycin), cytarabine (cytosine arabinoside), gemcitabine, 5-fluorouracil, hydroxyurea, elesclomol, etoposide, teniposide, amsacrine, mitoxantrone, camptothecin, topotecan, irinotecan, chlorambucil, cyclophosphamide, ifosfamide, melphalan, bortezomib, vincristine, vinblastine, vinorelbine, paclitaxel, docetaxel, iobitridol, iodipamide, iodixanol, iohexol, iomeprol, iopamidol, iopentol, iopromide, iotrolan, ioversol, ioxilan, iothalamate, ioxithalamate, ioxaglate, metrizamide, acetrizoate, metrizoate, diatrizoate, cyclosporine A, tacrolimus (FK506), sirolimus (rapamycin), everolimus, temsirolimus, zotarolimus, or biolimus.

In other embodiments, the adjunctive therapy includes treatment with methotrexate, carmustine, vincristine, paclitaxel, or thalidomide.

In some embodiments, the patient has a lymphoid or myeloid cancer.

In a eighth aspect, the invention features a PACAP polypeptide having an amino acid selected from a group consisting of Iaa, Iac, Ica, and Paa at position 1.

In an embodiment, the subject is a mammal (e.g., a human).

A person with ordinary skill in the art and a comprehensive knowledge of the structure-activity relationships of the PACAP analogs described herein, such as SEQ ID NOs: 4-78 (e.g., SEQ ID NOs: 4-14) would recognize that a very large number of other peptides with similar biological activity could be synthesized by making “conservative substitutions” (see DEFINITIONS) at one or more positions in these PACAP analogs (e.g., SEQ ID NOs: 4 to 14). For example, Onoue et al. (J Mol Neurosci 43:85-93, 2011) replaced Lys at positions 15, 20 and 21 in PACAP38 with Arg and Met at position 17 with Leu. The result of these four conservative substitutions was a new peptide with a solution structure and biological activity that was very similar to PACAP38. A person with ordinary skill in the art would recognize that the same four conservative substitutions made by Onoue et al. in PACAP38 could be made in any of the PACAP analogs described herein, such as SEQ ID NOs: 4-78 (e.g., SEQ ID NOs: 4-14) without any significant change in their biological activity. In fact, more than half of the amino acids in PACAP38 could probably be replaced conservatively without major changes in its biological activity. On the other hand, a single non-conservative replacement in a peptide can have a dramatic effect on its biological activity. For example, replacing Asp in position 3 of PACAP27 by Asn decreased the receptor binding affinity of the new peptide by more than 4,000-fold (Hou et al., Neuropharmacology 33:1189-1195, 1994). In addition, a single non-conservative replacement in a peptide can have an unexpected effect on the biological activity of the peptide. For example, replacing Ser in position 2 of PACAP38 by D-Pro converted the new peptide into a PAC₁ receptor antagonist (Bourgault et al., J Med Chem 52:3308-3316, 2009). The general formula (I) is based on structure-activity relationships of the PACAP analogs described herein, such as SEQ ID NOs: 4-78 (e.g., SEQ ID NOs: 4-14) and the generally accepted concepts outlined above.

These PACAP analogs of the invention, such as SEQ ID NOs: 4-78 (e.g., SEQ ID NOs: 4-14) may be used as prophylactic/therapeutic agents for a wide range of medical disorders in humans or other mammals, including (but not limited to) age-related neurodegenerative diseases (such as Alzheimer's disease, Parkinson's disease and amyotrophic lateral sclerosis), injuries to the central nervous system caused by stroke, heart attack and blunt force trauma (such as concussions and spinal cord trauma), Huntington's disease and other CAG codon repeat expansion diseases, retinal diseases (such as ischemia/reperfusion injury, non-infectious uveitis, diabetic retinopathy, macular degeneration, and glaucoma), autoimmune diseases (such as rheumatoid arthritis, Crohn's disease, ulcerative colitis, scleroderma, Sjögren's disease, idiopathic membranous nephropathy, Goodpasture's disease, autoimmune hepatitis, autoimmune myocarditis, myasthenia gravis, multiple sclerosis, Guillain-Barré syndrome, type I diabetes, Hashimoto's thyroiditis, Graves' disease, pemphigus vulgaris, and systemic lupus erythematosus), graft-versus-host disease, keratoconjunctivitis sicca caused by aging, autoimmune diseases, corneal transplantation, or keratorefractive surgery, type II diabetes, sepsis caused by bacteria and/or viruses (including bacterial and viral toxins), acute and chronic cardiovascular diseases (such as myocardial infarction, atherosclerosis, restenosis, and drug-induced cardiomyopathy), acute and chronic renal diseases (such as ischemia/reperfusion injury, nephritis and drug-induced nephrotoxicity), genetic disorders caused by premature in-frame stop codons, acute and chronic pulmonary diseases (such as asthma, chronic obstructive pulmonary disease, cystic fibrosis, and pulmonary arterial hypertension), systemic hypertension, hematological cancers (such as leukemias, lymphomas and plasma cell dyscrasias), DNA damage caused by therapeutic agents and radiation, eating disorders, acute and chronic liver diseases (such as ischemia/reperfusion injury, hepatitis and fatty liver), osteoporosis, pre-eclampsia, cell and solid organ transplantation, cognitive disorders, AIDS dementia complex, and aging of the central nervous system. The rationale and documentation for these diverse medical indications are described and documented below.

As life expectancy has increased, age-related neurodegenerative disorders, such as Alzheimer's disease, Parkinson's disease and amyotrophic lateral sclerosis, have become more prevalent and a greater burden on society. Published experiments indicate that PACAP-like peptides can protect neurons (neuroepithelial cells) in vitro against a very broad range of injuries (Arimura, Jpn J Physiol 48:301-331, 1998; Vaudry et al., Pharmacol Rev 61:283-357, 2009; Waschek, Br J Pharmacol 169:512-523, 2013). Published experiments using common in vivo preclinical models and human pathology specimens indicate that PACAP-like peptides would be efficacious for the treatment of Alzheimer's disease (Mercer et al., J Neurosci Res 76:205-215, 2004; Wu et al., Neurobiol Aging 27:377-386, 2006; Dogrukol-Ak et al., J Cereb Blood Flow Metab 29:411-422, 2009; Rat et al., FASEB J 25:3208-3218, 2011; Han et al., Neurology 82:1724-1728, 2014), Parkinson's disease (Delgado and Ganea., FASEB J 17:944-946, 2003; Reglödi et al., Behav Brain Res 151:303-312, 2004; Chung et al., Hum Mol Genet 14:1709-1725, 2005; Deguil et al., Neurotox Res. 17:142-155, 2010; Tunçel et al., J Mol Neurosci 46:51-57, 2012; Korkmaz et al., J Mol Neurosci 48:565-573, 2012; Brown et al., J Mol Neurosci 50:600-607, 2013; Watson et al., Neuroscience 240:277-286, 2013) and amyotrophic lateral sclerosis (Arimura et al., 1994; Nguyen et al., J Neurosci 24:1340-1349, 2004; Marden et al., J Clin Invest 117:2913-2919, 2007; Goursaud et al., FASEB J 25:3674-3686, 2011). In particular, the PACAP analogs of the invention, e.g., the polypeptides of SEQ ID NOs: 4-78 (e.g., SEQ ID NOs: 4-14), may be used to treat age-related neurodegenerative disorders, such as Alzheimer's disease, Parkinson's disease and amyotrophic lateral sclerosis.

Published experiments using common in vivo preclinical models indicate that PACAP-like peptides would be efficacious for the treatment of acute neurological diseases, including (but not limited to) stroke (Reglodi et al., Stroke 31:1411-1417, 2000; Chen et al., Regul Pept 137:4-19, 2006; Stetler et al., Proc Natl Acad Sci USA 107:3204-3209, 2010; Dejda et al., Peptides 32:1207-1216, 2011; Lazarovici et al., J Mol Neurosci 48:526-540, 2012), the central nervous system sequelae of heart attack (Uchida et al., Brain Res 736:280-286, 1996; Lenti et al., Brain Res 1283:50-57, 2009) and blunt force trauma to the brain and spinal cord (Farkas et al., Regul Pept 123:69-75, 2004; Chen and Tzeng, Neurosci Lett 384:117-121, 2005; Kövesdi et al., Neurotox Res 13:71-78, 2008; Johanson et al., J Neural Transm 118:115-133, 2011; Mao et al., J Neurotrauma 29:1941-1959, 2012; Tsuchikawa et al., J Mol Neurosci 48:508-517, 2012). These published experiments also suggest that PACAP-like peptides with high affinities for only the PAC₁ and VPAC₁ receptors would have higher therapeutic indices for the treatment of acute neurological diseases than PACAP-like peptides with high affinities for all three PACAP/VIP receptors. In particular, the PACAP analogs of the invention, e.g., the polypeptides of SEQ ID NOs: 4-78 (e.g., SEQ ID NOs: 4-14), may be used to treat acute neurological diseases, such as stroke, the central nervous system sequelae of heart attack, and blunt force trauma to the brain and spinal cord.

Huntington's disease is a fatal autosomal dominant disorder that is characterized by progressive cognitive and motor dysfunction. It is caused by expansion of the CAG codon (glutamine) repeat in the gene that codes for huntingtin. The neuropathological hallmark is the degeneration of neurons in the striatum. There are no effective treatments for Huntington's disease or the other CAG codon repeat diseases (such as spinobulbar muscular atrophy and the spinocerebellar ataxias). Published experiments using common in vivo preclinical models indicate that PACAP-like peptides would be efficacious for the treatment of Huntington's disease or other CAG codon repeat diseases (Emson et al., Brain Res 173:174-178, 1979; Tamás et al., Ann N Y Acad Sci 1070:570-574, 2006; Fahrenkrug et al., J Mol Neurosci 31:139-148, 2007; Pennuto et al., VIP, PACAP and Related Peptides [Tenth International Symposium], Eilat, 2011). In particular, the PACAP analogs of the invention, e.g., the polypeptides of SEQ ID NOs: 4-78 (e.g., SEQ ID NOs: 4-14), may be used to treat Huntington's disease.

Published experiments using common in vivo preclinical models indicate that PACAP-like peptides would be efficacious for the treatment of retinal diseases, including (but not limited to) ischemia/reperfusion injury (Szabadfi et al., Neurotox Res 21:41-48, 2012), non-infectious uveitis (Keino et al., Arch Ophthalmol 122:1179-1184, 2004; Camelo et al., J Ocul Pharmacol Ther 25:9-21, 2009), diabetic retinopathy (Giunta et al., Peptides 37:32-39, 2012; Szabadfi et al., Cell Tissue Res 348:37-46, 2012; Szabadfi et al., Neurochem Int 64:84-91, 2014), macular degeneration (Feret et al., Geriatr Nurs 28:387-392, 2007; Seki et al., J Mol Neurosci 36:57-60, 2008), and glaucoma (Silveira et al., J Biol Chem 277:16075-16080, 2002; Osborne et al., Prog Retin Eye Res 23:91-147, 2004; Atlasz et al., Gen Comp Endocrinol 153:108-114, 2007; Seki et al., J Mol Neurosci 36:57-60, 2008). In particular, the PACAP analogs of the invention, e.g., the polypeptides of SEQ ID NOs: 4-78 (e.g., SEQ ID NOs: 4-14), may be used to treat retinal diseases, such as ischemia/reperfusion injury, non-infectious uveitis, diabetic retinopathy, macular degeneration, and glaucoma.

Published experiments using common in vivo preclinical models indicate that PACAP-like peptides would also be efficacious for the treatment of autoimmune diseases, including (but not limited to) rheumatoid arthritis (Abad et al., J Immunol 167:3182-3189, 2001; Delgado et al., Nat Med 7:563-568, 2001), Crohn's disease (Abad et al., Gastroenterology 124:961-971, 2003; Arranz et al., Neuroimmunomodulation 15:46-53, 2008), ulcerative colitis, multiple sclerosis, Sjögren's disease, and type I diabetes. In addition, because of the overlapping mechanisms responsible for the pathogenesis of autoimmune diseases, PACAP-like peptides would be expected to be efficacious for the treatment of scleroderma, idiopathic membranous nephropathy, Guillain-Barré syndrome, Goodpasture's disease, autoimmune hepatitis, myasthenia gravis, Hashimoto's thyroiditis, Graves' disease, pemphigus vulgaris, and systemic lupus erythematosus. In particular, the PACAP analogs of the invention, e.g., the polypeptides of SEQ ID NOs: 4-78 (e.g., SEQ ID NOs: 4-14), may be used to treat autoimmune diseases, such as rheumatoid arthritis, Crohn's disease, ulcerative colitis, multiple sclerosis, Sjögren's disease, autoimmune myocarditis, and type I diabetes. In addition, the PACAP analogs of the invention, e.g., the polypeptides of SEQ ID NOs: 4-78 (e.g., SEQ ID NOs: 4-14), may also be used to treat scleroderma, idiopathic membranous nephropathy, Guillain-Barré syndrome, Goodpasture's disease, autoimmune hepatitis, myasthenia gravis, Hashimoto's thyroiditis, Graves' disease, pemphigus vulgaris, and systemic lupus erythematosus.

Keratoconjunctivitis sicca (dry eye syndrome) is an eye disorder that is caused by decreased tear production or increased tear evaporation, with decreased tear production being far more common. The most common cause of decreased tear production is aging. There are numerous other causes for decreased tear production, including hyposecretion of the lacrimal gland due to destruction, therapeutic agents (such as atropine, tricyclic antidepressants and morphine) or post-radiation fibrosis, and hyposecretion of the lacrimal gland associated with systemic autoimmune diseases (such as Wegener's granulomatosis, systemic lupus erythematosus and, especially, Sjögren's disease). Dry eye is also a common side-effect of keratorefractive surgery such as laser-assisted in situ keratomileusis (LASIK) and photorefractive keratectomy (Lee et al., Invest Ophthalmol Vis Sci 43:3660-3664, 2002). Published experiments using common in vitro and in vivo preclinical models indicate that PACAP-like peptides would also be efficacious for the treatment of keratoconjunctivitis sicca (dry eye syndrome) caused by aging, autoimmune diseases or keratorefractive surgery. PACAP-like peptides would have several distinct advantages over the immunosuppressive agents commonly used for the treatment of the dry eye syndrome. First, cyclosporine A emulsions usually take weeks to significantly increase tear production and months to reach their maximal effects (Utine et al., Ocul Immunol Inflamm 18:352-361, 2010), while PACAP-containing eye drops would significantly increase tear production as soon as 15 minutes after application (Nakamachi et al., VIP, PACAP and Related Peptides [Ninth International Symposium], Kagoshima, 2009). The rapid increase in tear production is due to the translocation of aquaporin 5 from the cytosol to the plasma membrane of acinar cells in the lacrimal gland (Nakamachi et al., VIP, PACAP and Related Peptides [Eleventh International Symposium], Pécs, 2013). Second, PACAP-containing eye drops would stimulate reinnervation of the cornea by the trigeminal nerve following keratorefractive surgery or corneal transplantation (Fukiage et al., Am J Ophthalmol 143:255-262, 2007; Nakajima et al., Mol Vis 19:174-183, 2013). Third, PACAP-like peptides would have a direct protective effect on corneal cells (Koh and Waschek, Ophthalmol Vis Sci 41:4085-4092, 2000; Nakamachi et al., VIP, PACAP and Related Peptides [Eleventh International Symposium], Pécs, 2013). These published experiments also suggest that PACAP-like peptides with a high affinity for only the PAC₁ receptor would have higher therapeutic indices for the treatment of the dry eye syndrome than PACAP-like peptides with high affinities for all three PACAP/VIP receptors. In particular, the PACAP analogs of the invention, e.g., the polypeptides of SEQ ID NOs: 4-78 (e.g., SEQ ID NOs: 4-14), may be used to treat keratoconjunctivitis sicca (dry eye syndrome). Specifically, eye drops used to treat keratoconjunctivitis sicca (dry eye syndrome) may contain any one of the PACAP analogs of the invention, e.g., any one of the polypeptides of SEQ ID NOs: 4-78 (e.g., SEQ ID NOs: 4-14).

The β-cells of the pancreas express both the PAC₁ receptor and the VPAC₂ receptor (Ahrén, Ann NY Acad Sci 1144:28-35, 2008). PAC₁ receptor-deficient mice had reduced glucose-stimulated insulin secretion and reduced glucose tolerance compared to wild-type mice (Jamen et al., J Clin Invest 105:1307-1315, 2000), while mice chronically treated with the PAC₁ receptor-specific agonist maxadilan had increased basal plasma levels of insulin and increased glucose tolerance compared to saline-treated mice (Yu et al., Peptides 29:1347-1353, 2008). Mice continuously infused with the VPAC₂ receptor-selective agonist BAY 55-9837 had increased basal plasma levels of insulin and increased glucose tolerance compared to saline-treated mice (Tsutsumi et al., Diabetes 51:1453-1460, 2002). These published experiments indicate that PACAP-like peptides would be efficacious for the treatment of type II diabetes. These published experiments also suggest that PACAP-like peptides with high affinities for only the PAC₁ and VPAC₂ receptors would have higher therapeutic indices for the treatment of type II diabetes than PACAP-like peptides with high affinities for all three PACAP/VIP receptors. In particular, the PACAP analogs of the invention, e.g., the polypeptides of SEQ ID NOs: 4-78 (e.g., SEQ ID NOs: 4-14), may be used to treat type II diabetes.

Published experiments using common in vivo preclinical models indicate that PACAP-like peptides would be efficacious for the treatment of sepsis caused by bacteria and/or viruses, including bacterial and viral toxins (Delgado et al., J Immunol 162:1200-1205, 1999; Martinez et al., Proc Natl Acad Sci USA 99:1053-1058, 2002, Martinez et al., J Leukoc Biol 77:729-738, 2005; Chorny and Delgado, Am J Pathol 172:1297-1307, 2008; Lv et al., Shock 31:185-191, 2009; Tang et al., Int Immunopharmacol 8:1646-1651, 2008). In particular, the PACAP analogs of the invention, e.g., the polypeptides of SEQ ID NOs: 4-78 (e.g., SEQ ID NOs: 4-14), may be used to treat sepsis caused by bacteria and/or viruses.

Published experiments using common in vitro and in vivo preclinical models indicate that PACAP-like peptides would also be efficacious for the treatment of a wide range of acute and chronic cardiovascular diseases, including (but not limited to) myocardial infarction, atherosclerosis, restenosis, and drug-induced cardiomyopathy caused by commonly used therapeutic agents such as doxorubicin and mitoxantrone. In particular, the PACAP analogs of the invention, e.g., the polypeptides of SEQ ID NOs: 4-78 (e.g., SEQ ID NOs: 4-14), may be used to treat acute and chronic cardiovascular diseases, such as myocardial infarction, atherosclerosis, restenosis, and drug-induced cardiomyopathy.

Published experiments using common in vitro and in vivo preclinical models indicate that PACAP-like peptides would be efficacious for the treatment of a wide range of acute and chronic renal diseases, including (but not limited to) disorders caused by ischemia/reperfusion, diabetes, light-chain immunoglobulin overload, and many commonly used therapeutic agents such as gentamicin, cisplatin, cyclosporine A, and radiocontrast agents. In particular, the PACAP analogs of the invention, e.g., the polypeptides of SEQ ID NOs: 4-78 (e.g., SEQ ID NOs: 4-14), may be used to treat acute and chronic renal diseases, such as disorders cased by ischemia/reperfusion, diabetes, light-chain immunoglobulin overload, and many commonly used therapeutic agents such as gentamicin, cisplatin, cyclosporine A, and radiocontrast agents.

There are more than 1,000 human genetic diseases that are caused in part by premature in-frame stop codons that result in the synthesis of truncated nonfunctional proteins. Burke and Mogg (Nucleic Acids Res 13:6265-6272, 1985) discovered that aminoglycosides could impair the recognition of premature stop codons in mammalian cells. Aminoglycosides have now been shown to impair the recognition of premature stop codons and to promote the synthesis of small quantities of full-length functional proteins in animal models and/or human clinical trials for numerous genetic diseases, including cystic fibrosis, Duchenne muscular dystrophy, Hurler's syndrome, nephropathic cystinosis, retinitis pigmentosa, and ataxia telangiectasia. However, the use of aminoglycosides to impair recognition of premature stop codons and promote the synthesis of full-length functional proteins is limited by their nephrotoxic (Mingeot-Leclercq and Tulkens, Antimicrob Agents Chemother 43:1003-1012, 1999) and ototoxic (Selimoglu, Curr Pharm Des 13:119-126) side-effects. PACAP-like peptides would be useful as adjunctive agents in combination with aminoglycosides for the treatment of genetic diseases caused by in-frame premature stop codons because of their cytoprotective effects against aminoglycoside-induced renal proximal tubule epithelial cell injury (Coy et al., PCT/US2010/055164, 2010). The combination of an aminoglycoside and a PACAP-like peptide should also be especially efficacious in a significant subset of recessive diseases where PACAP-like peptides would be expected to have modest efficacy as monotherapeutics, including (but not limited to) cystic fibrosis (see next paragraph), Duchenne muscular dystrophy, Krabbe's disease (globoid cell leukodystrophy), nephropathic cystinosis, and polycystic kidney disease. In particular, the PACAP analogs of the invention, e.g., the polypeptides of SEQ ID NOs: 4-78 (e.g., SEQ ID NOs: 4-14), may be used to treat genetic diseases that are caused in part by premature in-frame stop codons that result in the synthesis of truncated nonfunctional proteins, such as cystic fibrosis, Duchenne muscular dystrophy, Krabbe's disease (globoid cell leukodystrophy), Hurler's syndrome, nephropathic cystinosis, polycystic kidney disease, retinitis pigmentosa, and ataxia telangiectasia.

Published experiments using common in vivo preclinical models indicate that PACAP-like peptides would be efficacious for the treatment of a wide range of acute and chronic pulmonary diseases, including (but not limited to) asthma, chronic obstructive pulmonary disease, cystic fibrosis, and pulmonary arterial hypertension. In particular, the PACAP analogs of the invention, e.g., the polypeptides of SEQ ID NOs: 4-78 (e.g., SEQ ID NOs: 4-14), may be used to treat acute and chronic pulmonary diseases, such as asthma, chronic obstructive pulmonary disease, cystic fibrosis, and pulmonary arterial hypertension.

Systemic hypertension is a polygenic disease. Polymorphisms in the PACAP gene appear to represent predispositions for the development of systemic hypertension (Rutherford et al., Am J Med Genet A 126:241-247, 2004). In particular, the PACAP analogs of the invention, such as the polypeptides of SEQ ID NOs: 4-78 (e.g., SEQ ID NOs: 4-14), may be used to treat systemic hypertension.

Cancer is the leading cause of death in industrialized countries. Chemotherapy is the preferred treatment for disseminated cancers and metastatic tumors. Chemotherapy is also frequently used when surgery or radiation therapy have not completely eradicated a localized tumor, or as an adjunctive treatment with surgery or radiation therapy. Published experiments using common in vitro and in vivo preclinical models indicate that PACAP-like peptides would also be efficacious for the treatment of hematological cancers, including (but not limited to) blood cancers such as lymphoid and myeloid leukemias, lymphomas and plasma cell dyscrasias (such as multiple myeloma, Waldenström's macroglobulinemia, POEMS syndrome, Seligman's disease, and Franklin's disease). The published literature suggests that PACAP-like peptides inhibit the proliferation of most normal hematopoietic cells. PACAP-like peptides have been shown to inhibit the proliferation of HEL myeloid leukemia cells. One of the inventors of the present invention and his colleagues have shown that PACAP-like peptides potently inhibit the proliferation of multiple myeloma cells. One of the inventors of the present invention and his colleagues have also shown that PACAP-like peptides are efficacious in a patient with multiple myeloma. The inventors of the present invention have recently shown that PACAP-like peptides enhance the killing of both lymphoid and myeloid hematopoietic cancer cells by the commonly used anticancer agents carmustine, vincristine and thalidomide. Therefore, PACAP-like peptides would be efficacious for the treatment of lymphoid and myeloid hematopoietic cancers both as monotherapeutics and as adjunctive therapeutics with commonly used anticancer agents. PACAP-like peptides should also be efficacious for the treatment of lymphoid and myeloid hematopoietic cancers as adjunctive therapeutics with monoclonal antibody-small molecule anticancer agent conjugates, including (but not limited to) gemtuzumab ozogamicin, brentuximab vedotin, inotuzumab ozogamicin, RG-7596, pinatuzumab vedotin (RG-7593), SAR-3419, and BT-062. In particular, the PACAP analogs of the invention, such as the polypeptides of SEQ ID NOs: 4-78 (e.g., SEQ ID NOs: 4-14), may be used to treat cancer, such as hematological cancers, including (but not limited to) blood cancers such as lymphoid and myeloid leukemias, lymphomas and plasma cell dyscrasias (such as multiple myeloma, Waldenström's macroglobulinemia, POEMS syndrome, Seligman's disease, and Franklin's disease). Specifically, the PACAP analogs of the invention, such as the polypeptides of SEQ ID NOs: 4-78 (e.g., SEQ ID NOs: 4-14), may be used as monotherapeutics and as adjunctive therapeutics with commonly used anticancer agents.

In contrast, the published literature suggests that PACAP-like peptides promote the proliferation and survival of most (though not all) epithelial cancer cells. Oka et al. (Amer J Pathol 155:1893-1900, 1999) reported that PACAP protects HP75 human pituitary adenoma cells against apoptotic cell death caused by treatment with transforming growth factor-β1, and PACAP has been shown more recently to protect PC-3 androgen-independent human prostate cancer cells (Gutiérrez-Cañas et al., Br J Pharmacol 139:1050-1058, 2003) and CRL-2768 rat schwannoma cells (Castorina et al., Brain Res 1241:29-35, 2008) against apoptotic cell death caused by serum withdrawal. Onoue et al. (FEBS J 275:5542-5551, 2008) have shown that PACAP protects RIN-m5F insulinoma cells against apoptotic cell death caused by the anticancer agent streptozotocin. In addition, PACAP(6-38), a PACAP/VIP receptor antagonist, inhibited the growth in nude mice of xenografts of PC-3 human prostate cancer cells (Leyton et al., Cancer Lett 125:131-139, 1998), NCI-H838 human non-small cell lung cancer cells (Zia et al., Cancer Res 55:4886-4891, 1995) and MCF-7 human breast cancer cells (Leyton et al., Breast Cancer Res Treat 56:177-186, 1999). Therefore, parenteral administration of PACAP-like peptide, such as any one of the PACAP analogs of the invention, such as any one of the polypeptides of SEQ ID NOs: 4-78 (e.g., SEQ ID NOs: 4-14), is preferably avoided in patients with most (though perhaps not all) solid epithelial tumors. However, parenteral administration of PACAP/VIP receptor antagonists could be used to treat patients with solid epithelial tumors in combination with anticancer agents whose dose-limiting toxicity was myelosuppression.

Published experiments using common in vitro and in vivo preclinical models indicate that PACAP-like peptides would be efficacious for the prevention of DNA damage caused by commonly used therapeutic or anticancer agents (Li et al., Peptides 31:592-602, 2010; Stetler et al., Proc Natl Acad Sci USA 107:3204-3209, 2010; Khan et al., Physiol Rep 1:e00163, 2013). PACAP-like peptides should also be efficacious for the prevention of DNA damage caused by radiation. In particular, the PACAP analogs of the invention, such as the polypeptides of SEQ ID NOs: 4-78 (e.g., SEQ ID NOs: 4-14), may be used to prevent DNA damage caused by commonly used therapeutic or anticancer agents and radiation.

Glucocorticoids are frequently used for the treatment of patients with blood cancers and autoimmune diseases in order to inhibit the activity of B- and T-lymphocytes. However, a significant portion of the patients treated with glucocorticoids eventually become resistant to the steroid (Barnes and Adcock, Lancet 373:1905-1917, 2009). The inventors of the present invention have shown that PACAP-like peptides can still inhibit the proliferation of B-lymphocytes from a patient with multiple myeloma who had been treated for with a dexamethasone-containing regimen (Greenstein et al., Exp Hematol 31:271-282, 2003) even after the B-lymphocytes had become resistant to dexamethasone (Coy et al., PCT/US2010/055164, 2010). These experiments indicate that PACAP-like peptides, such as the PACAP analogs of the invention, e.g., the polypeptides of SEQ ID NOs: 4-78 (e.g., SEQ ID NOs: 4-14), may be efficacious in patients with blood cancers and autoimmune diseases even after the patients have become resistant to glucocorticoids. Therefore, these PACAP analogs of the invention, such as the polypeptides of SEQ ID NOs: 4-78 (e.g., SEQ ID NOs: 4-14), could be used to replace the glucocorticoid in common multiple drug regimens, such as COP (cyclophosphamide, Oncovin [vincristine] and prednisone) and VAD (vincristine, Adriamycin [doxorubicin] and dexamethasone), as soon as the patient becomes insensitive to the steroid.

Published experiments using common in vivo preclinical models indicate that PACAP-like peptides, such as the PACAP analogs of the invention, such as the polypeptides of SEQ ID NOs: 4-78 (e.g., SEQ ID NOs: 4-14), may be efficacious for the treatment of eating disorders.

Published experiments using common in vitro and in vivo preclinical models indicate that PACAP-like peptides, such as the PACAP analogs of the invention, such as the polypeptides of SEQ ID NOs: 4-78 (e.g., SEQ ID NOs: 4-14), may be efficacious for the treatment of a wide range of acute and chronic liver diseases, including (but not limited to) ischemia/reperfusion injury, hepatitis, and fatty liver.

Osteoporosis is characterized by a reduction in the mineral density of bone and, consequently, an increased risk of bone fractures. It is more common in women than men, especially in postmenopausal women. Osteoporosis is also a common side-effect of many glucocorticoid-containing anticancer therapeutic regimens. Published experiments using common in vitro preclinical models indicate that PACAP-like peptides, such as the PACAP analogs of the invention, such as the polypeptides of SEQ ID NOs: 4-78 (e.g., SEQ ID NOs: 4-14), may be efficacious for the treatment of osteoporosis.

Pre-eclampsia is a life-threatening disorder that occurs during 5-10% of pregnancies, usually during the second and third trimester. Pre-eclampsia involves damage to the placental endothelium, kidneys and liver. The principal symptoms are systemic hypertension, inflammation and elevated levels of protein in the urine. Published clinical experiments and experiments using common in vitro and in vivo preclinical models indicate that PACAP-like peptides, such as the PACAP analogs of the invention, such as polypeptides of SEQ ID NOs: 4-78 (e.g., SEQ ID NOs: 4-14), may be efficacious for the treatment of pre-eclampsia.

Published experiments using common preclinical models indicate that PACAP-like peptides, such as the PACAP analogs of the invention, such as the polypeptides of SEQ ID NOs: 4-78 (e.g., SEQ ID NOs: 4-14), may enhance the in vitro preservation and in vivo survival of cells and solid organs during storage and transplantation.

A PACAP-like peptide plays important roles in learning and memory in Arthropods. Published experiments in mammals using common in vivo preclinical models indicate that PACAP-like peptides, such as the PACAP analogs of the invention, e.g., the polypeptides of SEQ ID NOs: 4-78 (e.g., SEQ ID NOs: 4-14), may be efficacious for the treatment of cognitive disorders during normal aging.

The AIDS dementia complex (HIV encephalopathy) is a severe cognitive and motor disorder caused by infection of microglial cells in the brain by the human immunodeficiency virus. The pathological features include microglial activation, neuronal apoptosis and demyelination. Stimulation of the VPAC₂ receptor has been shown to inhibit integration of the human immunodeficiency virus into genomic DNA. In addition, PACAP has been shown to protect cortical neurons against the toxic effects of the envelope glycoprotein of the human immunodeficiency virus gp120 and to “deactivate” activated microglial cells. In particular, the PACAP analogs of the invention, e.g., the polypeptides of SEQ ID NOs: 4-78 (e.g., SEQ ID NOs: 4-14), may be used to treat AIDS dementia complex (HIV encephalopathy)

Normal aging of the central nervous system is accompanied by an increase in the levels of proinflammatory cytokines and superoxide, and a decrease in both the number of basal forebrain cholinergic neurons and the rate of proliferation of neural progenitor cells in the subependymal zone of the dentate gyrus. Reduction of extracellular superoxide levels and stimulation of neural progenitor cell proliferation in the subependymal zone of the dentate gyrus improves cognitive performance during aging. PACAP has been shown to protect basal forebrain cholinergic neurons against apoptosis, inhibit inflammation, reduce extracellular superoxide levels, stimulate the proliferation of neural progenitor cells in the subependymal zone of the dentate gyrus, and enhance learning and memory. Therefore, PACAP would be efficacious for reversing the cognitive and motor decline during normal aging. In particular, the PACAP analogs of the invention, e.g., the polypeptides of SEQ ID NOs: 4-78 (e.g., SEQ ID NOs: 4-14), may be used to treat age-related diseases and to enhance learning and memory.

The PACAP analogs and compounds of the invention, such as the polypeptides of SEQ ID NOs: 4-78 (e.g., SEQ ID NOs: 4-14), may be coupled to suitable radionuclides and used in the localization, diagnosis and treatment of disseminated cancers and metastatic tumors in humans or other mammals, and/or coupled to small molecule therapeutics and used as vectors for targeted drug delivery to humans or other mammals. Examples of suitable radionuclides that can be coupled to the PACAP analogs of the invention (e.g., one or more of the PACAP analogs having the sequence of Formula (I) (such as the sequence of SEQ ID NOs: 4-78 (e.g., SEQ ID NOs: 4-14))) include, e.g., a γ-emitting radionuclide, Auger-emitting radionuclide, β-emitting radionuclide, an α-emitting radionuclide, or a positron-emitting radionuclide (e.g., ¹¹C, ¹³N, ¹⁵O, ¹⁸F, ⁵²Fe, ⁵⁵Co, ⁶¹Cu, ⁶²Cu, ⁶⁴Cu, ⁶⁷Cu, ⁶⁷Ga, ⁶⁸Ga, ⁶²Zn, ⁶³Zn, ⁷⁰As, ⁷¹As, ⁷⁴As, ⁷⁶Br, ⁷⁹Br, ⁸²Rb, ⁸⁶Y, ⁸⁹Zr, ¹¹⁰In, ¹¹¹In, ¹²⁰I, ¹²³I, ¹²⁴I, ¹²⁵I, ¹³¹I, ¹²²Xe, ¹⁷⁵Lu, ¹⁵⁴Gd, ¹⁵⁵Gd, ¹⁵⁶Gd, ¹⁵⁷Gd, ¹⁵⁸Gd, ^{94m}Tc, ⁹⁴Tc, and ^{99m}Tc). The PACAP analogs and compounds of the invention can also be coupled to suitable imaging agents and used in the localization of granulomas in humans or other mammals with various infectious or autoimmune diseases. Paramagnetic metals may be used in conjunction with the PACAPanalogs of the invention. Small molecule therapeutics and anticancer agents that could be coupled to the PACAP analogs of the invention are described below.

The maximal tolerable dose of the most commonly used cancer therapeutics is limited by their toxic effects on one or more major organs of the body of humans or other mammals. For example, the dose-limiting toxicity for cancer chemotherapy with cisplatin is nephrotoxicity, the dose-limiting toxicity for cancer chemotherapy with bleomycin is pulmonary toxicity, and the dose-limiting toxicity for cancer chemotherapy with doxorubicin is cardiotoxicity. Several strategies have been used to increase the maximal tolerable dose of cancer therapeutics and, thus, increase their therapeutic effectiveness. For example, cancer therapeutics have been conjugated to monoclonal antibodies directed against tumor-associated antigens or to bioactive peptides whose receptors are highly expressed in selected types of tumors in order to preferentially deliver the anticancer agent to the interior of tumor cells. An alternate strategy to increase the efficacy of cancer therapeutics is to preferentially protect normal tissues against the cytotoxic effects of the anticancer agents. In particular, the PACAP analogs of the invention, e.g., the polypeptides of SEQ ID NOs: 4-78 (e.g., SEQ ID NOs: 4-14), may be used to reduce the dose-limiting toxicity caused by anticancer agents, such as cisplatin, bleomycin, doxorubicin.

The U.S. FDA has approved several cytoprotective agents for use with anticancer agents, including amifostine (Ethyol), dexrazoxane (Zinecard) and mesna (Mesenex). None of these cytoprotective agents acts via G-protein-coupled receptors. In particular, the PACAP analogs of the invention, such as the polypeptides of SEQ ID NOs: 4-78 (e.g., SEQ ID NOs: 4-14), may be used as cytoprotective agents in combination with anticancer agents.

Accordingly, the present invention also relates to methods and compositions for the treatment, management, and prevention of injuries to the major organs of the body, such as nervous system, brain, spinal cord, heart, lung, kidneys, liver, pancreas, gall bladder, gastrointestinal tract, adrenal gland, thymus, spleen, lymph nodes, breast, ovary, testes, cornea, and prostate, preferably nervous system, heart, lung, kidneys, liver, cornea, and gastrointestinal tract, of humans or other mammals caused by trauma, acute or chronic diseases, or one or more prophylactic/therapeutic agents. The method comprises administering an effective amount of one or more of the above PACAP analogs of the invention, e.g., the polypeptides of SEQ ID NOs: 4-78 (e.g., SEQ ID NOs: 4-14), having activities at one or more PACAP/VIP receptors, for the inhibition of the pathology caused by trauma, chronic diseases, or one or more prophylactic/therapeutic agents.

PACAP analogs are extremely effective in protecting neurons, cardiomyocytes, hepatocytes, and lung, kidney and gastrointestinal epithelial cells in a concentration-dependent manner. Thus, the present invention relates to a method of treatment of these cells by administering a concentration of about 10⁻¹³ M to 10⁻⁶ M of any one of the PACAP analogs of the invention, such as any one of the polypeptides of SEQ ID NOs: 4-78 (e.g., SEQ ID NOs: 4-14). When these cells are in culture, the concentration of the PACAPanalog is preferably between 10⁻¹³ M and 10⁻⁶ M in the culture medium. When these cells are in the organs of a subject, the concentration of the PACAP analog of the invention, such as any one of the polypeptides of SEQ ID NOs: 4-78 (e.g., SEQ ID NOs: 4-14), is preferably between about 10⁻¹³M to 10⁻⁶ M in the interstitial space or blood. Within the generally effective concentration range of the compositions of this invention, there is a peak effectiveness, below which the effectiveness of the composition falls off to a significant degree. In a preferred embodiment, the concentration of the PACAP analog of the present invention is between about 10⁻¹³ M and about 10⁻⁶ M, which permits treatment of the subject with minimal risk of adverse side effects from the treatment. In a preferred embodiment, the concentration of the PACAP analog of the invention, such as any one of the polypeptides of SEQ ID NOs: 4-78 (e.g., SEQ ID NOs: 4-14), is about 10⁻⁹ M. The present discovery makes possible the use of the compositions of this invention in low concentrations to provide substantial protection of neurons, cardiomyocytes, hepatocytes, and lung, kidney and gastrointestinal epithelial cells. In a specific embodiment, the composition of the present invention protects these cells from injury or death. The injury or death of these cells may be due to trauma, chronic diseases, or one or more prophylactic/therapeutic agents.

The compositions of the invention may be administered intravenously, intraperitoneally, subcutaneously, intramuscularly, or otherwise into the bloodstream in order to achieve the optimal concentration for the treatment, management or prevention of injuries to one or more of the major organs of the body of humans or other mammals caused by treatment with one or more therapeutic or anticancer agents. The intravenous administration of the composition of the present invention may be as a bolus injection, as a constant infusion or as a bolus injection followed immediately by a constant infusion. In a preferred embodiment, the subject is being treated with one or more anticancer agents for a hematological cancer, and the PACAP analog of the invention, such as any one of the polypeptides of SEQ ID NOs: 4-78 (e.g., SEQ ID NOs: 4-14), is administered as a bolus injection (in order to saturate any serum binding proteins) followed immediately by a constant infusion.

The compositions of the invention may be administered by inhalation or intranasally in order to have preferential access to the lung or the brain, respectively. In a preferred embodiment, the subjects are treated by inhalation with one or more of the PACAP analogs of the invention, such as the polypeptides of SEQ ID NOs: 4-78 (e.g., SEQ ID NOs: 4-14), for pulmonary arterial hypertension. In another preferred embodiment, the subjects are treated intranasally with one or more of the PACAP analogs of the invention, such as the polypeptides of SEQ ID NOs: 4-78 (e.g., SEQ ID NOs: 4-14), for a concussion.

The compositions of the invention may be administered orally in a time-dependent or a pH-dependent formulation in order to have preferential access to different levels of the gastrointestinal tract or an injured region of the gastrointestinal tract, respectively. In a preferred embodiment, the subjects are treated with one or more of the PACAP analogs of the invention, such as the polypeptides of SEQ ID NOs: 4-78 (e.g., SEQ ID NOs: 4-14), for Crohn's disease or ulcerative colitis.

The compositions of the invention may be administered in a controlled-release or a sustained-release formulation. In a preferred embodiment, the subjects are treated with one or more therapeutic agents for a hematological cancer or an autoimmune disease.

The compositions of the invention may be administered after encapsulation in liposomes. In particular, the PACAP analogs of the invention, such as the polypeptides of SEQ ID NOs: 4-78 (e.g., SEQ ID NOs: 4-14), may be administered after encapsulation in liposomes

The compositions of the present invention may be administered transcutaneously after encapsulation in dendrimers. In a preferred embodiment, the subjects are treated with one or more therapeutic agents for a hematological cancer or an autoimmune disease.

The compositions of the present invention may be administered in combination with other cytoprotective adjunctive agents that have different mechanisms of action, such as amifostine, dexrazoxane, mesna, palifermin (human keratinocyte growth factor), apocynin, erythropoietin, N-acetylcysteine, and N-acetylcysteine amide in order to have an additive or a synergistic effect. In particular, the PACAP analogs of the invention, such as the polypeptides of SEQ ID NOs: 4-78 (e.g., SEQ ID NOs: 4-14), may be administered in combination with other cytoprotective adjunctive agents.

The compositions of the present invention may be used to treat, manage or prevent injuries to one or more major organs of the body of humans or other mammals caused by both unconjugated anticancer agents and anticancer agents reversibly conjugated to a monoclonal antibody or to one or more bioactive peptides.

The compositions of the present invention may be used to directly enhance the efficacy of some anticancer agents on some cancer cells, especially the anticancer activity of some chemotherapeutics on lymphoid and myeloid hematopoietic cancers.

The compositions of the present invention may be coupled to radionuclides to localize, diagnose and treat disseminated cancers and metastatic tumors.

The compositions of the present invention may be coupled to small molecule therapeutics to target the delivery of the therapeutics preferentially to specific tissues or cell types.

The compositions of the present invention may be coupled to suitable imaging agents to localize granulomas in humans or other mammals with various infectious or autoimmune diseases.

The compositions of the present invention may be used to coat metallic or biodegradable stents to prevent restenosis of coronary arteries or other large arteries.

### BRIEF DESCRIPTION OF THE DRAWINGS

FIG. 1 shows the primary amino acid sequences of PACAP38 (SEQ ID NO:1), PACAP27 (SEQ ID NO:2), VIP (SEQ ID NO:3), [Iac¹]PACAP38 (SEQ ID NO:4), [Iaa¹]PACAP38 (SEQ ID NO:5), [Iaa¹,Ala^{16,17},D-Lys³⁸]PACAP38 (SEQ ID NO:6), [Iaa¹,D-Ser²]PACAP38 (SEQ ID NO:7), [Iac¹,Ala²²]PACAP38 (SEQ ID NO:8), [Iaa¹,Ala²²]PACAP38 (SEQ ID NO:9), [Paa¹,Ala²²]PACAP38 (SEQ ID NO:10), [Iaa¹,D-Ser²,Ala²²]PACAP38 (SEQ ID NO:11), [Iaa¹,D-Tyr²,Ala²²]PACAP38 (SEQ ID NO:12), [Iaa¹,D-Ala²,Ala²²]PACAP38 (SEQ ID NO:13), and [Iac¹,D-Ser²,Ala²²]PACAP38 (SEQ ID NO:14).

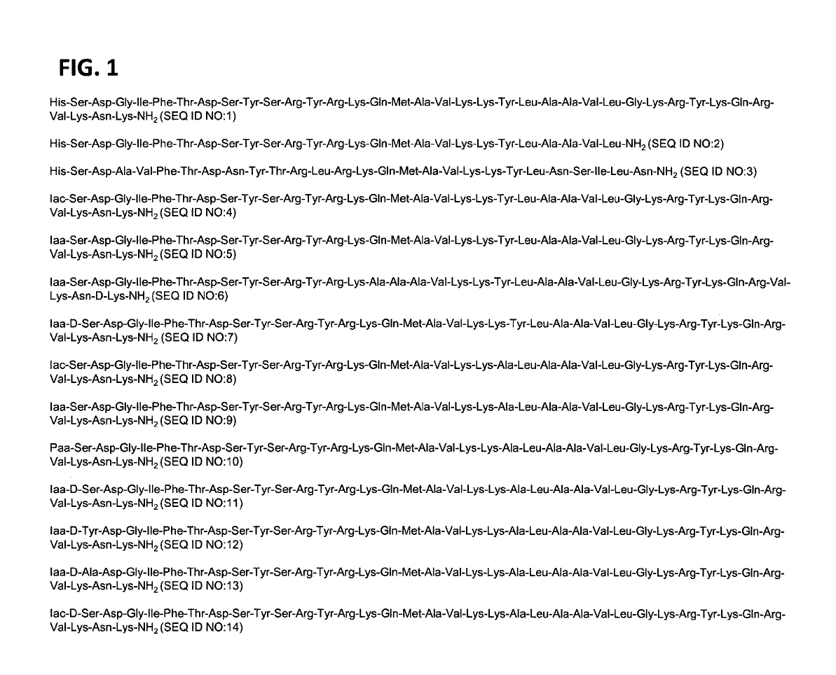

FIG. 2 is table comparing the molecular weights of eleven PACAP analogs of the invention (SEQ ID NOs 4-78) as determined by matrix-assisted laser desorption/ionization (MALDI) mass spectroscopy (MS) using an Applied Biosystems Voyager DE machine with the calculated molecular weight based on the amino acid composition.

FIG. 3 is a graph showing the reduction in cisplatin-induced increase in serum creatinine levels caused by administration of comparable amounts of PACAP38 and [Iac¹]PACAP38. Male C57BL/6 mice were given a single intraperitoneal injection of 20 mg/kg of cisplatin. Twenty micrograms of PACAP38 were given intraperitoneally 1 hour before the injection of cisplatin and additional doses were given at 24 and 48 hours after the initial dose. The control group of mice was injected intraperitoneally with the same volume of saline as for the injections of cisplatin and PACAP38 on the same schedule. The mice were euthanized 24 hours after the final injection of PACAP38. The serum creatinine levels were determined at sacrifice with isotope dilution liquid chromatography mass spectroscopy/mass spectroscopy. Each value represents the mean plus/minus the standard error of eight determinations. ** p<0.01 compared to the group treated only with cisplatin.

FIG. 4 is a graph showing the reduction in cisplatin-induced increase in serum cystatin C levels caused by administration of comparable amounts of PACAP38 and [Iac¹]PACAP38. Male C57BL/6 mice were given a single intraperitoneal injection of 20 mg/kg of cisplatin. Twenty micrograms of PACAP38 were given intraperitoneally 1 hour before the injection of cisplatin and additional doses were given at 24 and 48 hours after the initial dose. The control group of mice was injected intraperitoneally with the same volume of saline as for the injections of cisplatin and PACAP38 on the same schedule. The mice were euthanized 24 hours after the final injection of PACAP38. The serum cystatin C levels were determined at sacrifice with commercial ELISA kits. Each value represents the mean plus/minus the standard error of eight determinations. ** p<0.01 compared to the group treated only with cisplatin.

FIG. 5 is a graph showing the reduction in cisplatin-induced body weight loss caused by administration of comparable amounts of PACAP38 and [Iac¹]PACAP38. Male C57BL/6 mice were given a single intraperitoneal injection of 20 mg/kg of cisplatin. Twenty micrograms of PACAP38 were given intraperitoneally 1 hour before the injection of cisplatin and additional doses were given at 24 and 48 hours after the initial dose. The control group of mice was injected intraperitoneally with the same volume of saline as for the injections of cisplatin and PACAP38 on the same schedule. The mice were euthanized 24 hours after the final injection of PACAP38. The body weights of the mice were determined 1-2 hours before the first injection of PACAP38 and at sacrifice. Each value represents the mean plus/minus the standard error of eight determinations. ** p<0.01 and * p<0.05 compared to the group treated only with cisplatin.

FIG. 6 is a graph showing the reduction in cisplatin-induced increase in kidney levels of tumor necrosis factor-α (TNF-α) caused by administration of comparable amounts of PACAP38 and [Iac¹]PACAP38. Male C57BL/6 mice were given a single intraperitoneal injection of 20 mg/kg of cisplatin. Twenty micrograms of PACAP38 were given intraperitoneally 1 hour before the injection of cisplatin and additional doses were given at 24 and 48 hours after the initial dose. The control group of mice was injected intraperitoneally with the same volume of saline as for the injections of cisplatin and PACAP38 on the same schedule. The mice were euthanized 24 hours after the final injection of PACAP38. The kidney levels of TNF-α were determined at sacrifice with commercial ELISA kits. Each value represents the mean plus/minus the standard error of eight determinations. ** p<0.01 compared to the group treated only with cisplatin.

FIG. 7 is a graph showing the reduction in cisplatin-induced increase in kidney levels of interferon γ (IFN-γ) caused by administration of comparable amounts of PACAP38 and [Iac¹]PACAP38. Male C57BL/6 mice were given a single intraperitoneal injection of 20 mg/kg of cisplatin. Twenty micrograms of PACAP38 were given intraperitoneally 1 hour before the injection of cisplatin and additional doses were given at 24 and 48 hours after the initial dose. The control group of mice was injected intraperitoneally with the same volume of saline as for the injections of cisplatin and PACAP38 on the same schedule. The mice were euthanized 24 hours after the final injection of PACAP38. The kidney levels of IFN-γ were determined at sacrifice with commercial ELISA kits. Each value represents the mean plus/minus the standard error of eight determinations. ** p<0.01 and * p<0.05 compared to the group treated only with cisplatin.

FIG. 8 is a graph showing the reduction in cisplatin-induced increase in kidney levels of monocyte chemotactic protein 1 (MCP-1, CCL2) caused by administration of comparable amounts of PACAP38 and [Iac¹]PACAP38. Male C57BL/6 mice were given a single intraperitoneal injection of 20 mg/kg of cisplatin. Twenty micrograms of PACAP38 were given intraperitoneally 1 hour before the injection of cisplatin and additional doses were given at 24 and 48 hours after the initial dose. The control group of mice was injected intraperitoneally with the same volume of saline as for the injections of cisplatin and PACAP38 on the same schedule. The mice were euthanized 24 hours after the final injection of PACAP38. The kidney levels of MCP-1 were determined at sacrifice with commercial ELISA kits. Each value represents the mean plus/minus the standard error of eight determinations. ** p<0.01 and * p<0.05 compared to the group treated only with cisplatin.

FIG. 9 is a graph showing the changes in arterial blood pressure caused by [Iaa¹]PACAP38, [Iaa¹,D-Ser²]PACAP38, [Iaa¹,Ala²²]PACAP38, and [Iaa¹,D-Ser²,Ala²²]PACAP38. Male Sprague-Dawley rats (300-325 g) were anesthetized with 5% isoflurane for induction and then intubated. The rats were then ventilated (Hallowell EMC) with 100% O₂ and 1-2% isoflurane. A PE-50 cannula was then placed in the femoral artery and connected to a pressure transducer. The arterial pressure signal was sent to an electronic chart recorder (BioPac). Heart rate was electronically derived from the arterial pressure signal. A similar PE-50 cannula was also placed in the femoral vein to allow for intravenous injection of the test peptides. Ten micrograms of the test peptides were injected intravenously in 100 μl of 0.9% NaCl in a random order and the cannula was immediately flushed with 100 μl of 0.9% NaCl. Changes in blood pressure and heart rate were calculated from the electronic record. Intravenous injection of [Iaa¹]PACAP38, [Iaa¹,D-Ser²]PACAP38 or [Iaa¹,Ala²²]PACAP38 produced a large decrease in arterial blood pressure but only [Iaa¹]PACAP38 or [Iaa¹,D-Ser²]PACAP38 produced a decrease in arterial pulse pressure, which was much more dramatic for [Iaa¹]PACAP38. Intravenous injection of [Iaa1]PACAP38 or [Iaa1,Ala22]PACAP38 also produced an increase in the heart rate from 378 bpm to 425 bpm or from 415 bpm to 440 bpm, respectively. Intravenous injection of [Iaa¹,D-Ser²,Ala²²]PACAP38 produced a slight increase in the arterial blood pressure and an increase in the heart rate from 410 bpm to 457 bpm.

FIG. 10 is a graph showing the changes in arterial blood pressure caused by [Iac¹]PACAP38, [Iac¹,Ala²²]PACAP38, [Iac¹,D-Ser²,Ala²²]PACAP38, and [Iaa¹,Ala^{16,17},D-Lys³⁸]PACAP38. Male Sprague-Dawley rats (300-325 g) were anesthetized with 5% isoflurane for induction and then intubated. The rats were then ventilated (Hallowell EMC) with 100% O₂ and 1-2% isoflurane. A PE-50 cannula was then placed in the femoral artery and connected to a pressure transducer. The arterial pressure signal was sent to an electronic chart recorder (BioPac). Heart rate was electronically derived from the arterial pressure signal. A similar PE-50 cannula was also placed in the femoral vein to allow for intravenous injection of the test peptides. Ten micrograms of the test peptides were injected intravenously in 100 μl of 0.9% NaCl in a random order and the cannula was immediately flushed with 100 μl of 0.9% NaCl. Changes in blood pressure and heart rate were calculated from the electronic record. Intravenous injection of [Iac¹,Ala²²]PACAP38 or [Iac¹,D-Ser²,Ala²²]PACAP38 produced an increase in the heart rate from 416 bpm to 435 bpm or from 420 bpm to 441 bpm, respectively. Intravenous injection of [Iaa¹,Ala^{16,17},D-Lys³⁸]PACAP38 produced a large and prolonged decrease in both arterial blood pressure and pulse pressure and a large decrease in the heart rate.

### SEQUENCES

SEQ ID NO:1-3 are human sequences. SEQ ID NO: 4-78 are modifications of the corresponding human sequences. Below is a brief description of SEQ ID NOs: 1-14.

SEQ ID NO:1 is the amino-acid sequence of PACAP38.

SEQ ID NO:2 is the amino-acid sequence of PACAP27.

SEQ ID NO:3 is the amino-acid sequence of VIP.

SEQ ID NO:4 is the amino-acid sequence of [Iac¹]PACAP38, which can be used for the purposes described in the present invention.

SEQ ID NO:5 is the amino-acid sequence of [Iaa¹]PACAP38, which can be used for the purposes described in the present invention.

SEQ ID NO:6 is the amino-acid sequence of [Iaa¹,Ala^{16,17},D-Lys³⁸]PACAP38, which can be used for the purposes described in the present invention.

SEQ ID NO:7 is the amino-acid sequence of [Iaa¹,D-Ser²]PACAP38, which can be used for the purposes described in the present invention.

SEQ ID NO:8 is the amino-acid sequence of [Iac¹,Ala²²]PACAP38, which can be used for the purposes described in the present invention.

SEQ ID NO:9 is the amino-acid sequence of [Iaa¹,Ala²²]PACAP38, which can be used for the purposes described in the present invention.

SEQ ID NO:10 is the amino-acid sequence of [Paa¹,Ala²²]PACAP38, which can be used for the purposes described in the present invention.

SEQ ID NO:11 is the amino-acid sequence of [Iaa¹,D-Ser²,Ala²²]PACAP38, which can be used for the purposes described in the present invention.

SEQ ID NO:12 is the amino-acid sequence of [Iaa¹,D-Tyr²,Ala²²]PACAP38, which can be used for the purposes described in the present invention.

SEQ ID NO:13 is the amino-acid sequence of [Iaa¹,D-Ala²,Ala²²]PACAP38, which can be used for the purposes described in the present invention.

SEQ ID NO:14 is the amino-acid sequence of Iac¹,D-Ser²,Ala²²]PACAP38, which can be used for the purposes described in the present invention.

### DEFINITIONS

The following standard three-letter or four-letter abbreviations are used herein to identify amino acid or carboxylic acid residues.
Abu, α-aminobutyric acid
Acb, 1-amino-1-cyclobutanecarboxylic acid
Ach, 1-amino-1-cyclohexanecarboxylic acid
Acpe, 1-amino-1-cyclopentanecarboxylic acid
Acpr, 1-amino-1-cyclopropanecarboxylic acid
Aib, α-aminoisobutyric acid
Ala, alanine
Arg, arginine
Asn, asparagine
Asp, aspartic acid
Bip, 4,4′-biphenylalanine
Cha, cyclohexylalanine
Cys, cysteine
Dab, diaminobutyric acid
Dap, diaminopropionic acid
Dopa, 3,4-dihydroxyphenylalanine
Gaba, γ-amino-N-butyric acid
Gln, glutamine
Glu, glutamic acid
Gly, glycine
His, histidine
hSer, homoserine
Hyp, hydroxyproline
Iaa, imidazole-4-acetic acid
Iac, imidazole-4-acrylic acid
Ica, imidazole-4-carboxylic acid
Ile, isoleucine
Leu, leucine
Lys, lysine
N-Me-Asp, N-methylaspartic acid
N-Me-Ser, N-methylserine
Met, methionine
Nal, 1-naphthylalanine or 2-naphthylalanine
Nle, norleucine
Nva, norvaline
Orn, ornithine
Paa, 3-pyridylacetic acid
Pal, 2-pyridylalanine, 3-pyridylalanine or 4-pyridylalanine
Phe, phenylalanine
Pro, proline
Pse, phenylserine
Sar, sarcosine (N-methylglycine)
Ser, serine
Thr, threonine
Trp, tryptophan
Tyr, tyrosine
Val, valine

As used herein, the term “PACAP” refers to human PACAP27 (SEQ ID NO:2) and/or human PACAP38 (SEQ ID NO:1).

As used herein, the term “PACAP/VIP agonist” refers to any molecule, including a protein, naturally or synthetically post-translationally modified protein, polypeptide, naturally or synthetically modified polypeptide, peptide, naturally or synthetically modified peptide, and large or small nonpeptide molecule that binds to and stimulates one or more of the PACAP/VIP receptors.

As used herein, the term “analog” refers to both conformational and linear sequence analogs. Maxadilan, a 61-amino-acid peptide with two disulfide bridges that is synthesized naturally in the salivary glands of the hematophagous sand fly Lutzomyia longipalpis, is one example of a conformational analog of PACAP. It has no obvious linear amino-acid sequence identities with PACAP but binds preferentially to the PAC₁ receptors with high affinity (Tatsuno et al., Brain Res 889:138-148, 2001; Lerner et al., Peptides 28:1651-1654, 2007). The amino-acid sequences of maxadilan made by sand flies from different regions of Central and South America can differ by more than 20%. However, the relative positions of the cysteine residues in these bioactive orthologs are invariant and all of these bioactive orthologs have a similar predicted secondary structure. The amino-acid sequences of some naturally occurring maxadilans are described by Lanzaro et al. (Insect Mol Biol 8:267-275, 1999). Therefore, linear analogs of conformational analogs of PACAP, such as linear analogs of maxadilan (Reddy et al., J Biol Chem 281:16197-16201, 2006), would be expected to bind to and stimulate PACAP/VIP receptors. Those skilled in the art will recognize that additional conformational analogs of PACAP could be created by synthetic combinatorial chemistry or phage display technologies. A peptide analog may contain one or more amino acids that occur naturally in mammalian cells but do not occur naturally in mammalian peptides. For example (but not by way of limitation), a peptide analog may contain γ-amino-N-butyric acid (GABA), β-alanine, ornithine, and citrulline, imidazole-4-acetic acid (Iaa), imidazole-4-acrylic acid (Iac), imidazole-4-carboxylic acid (Ica), or 3-pyridylacetic acid (Paa). A peptide analog may also contain one or more nonnatural amino acids that do not occur naturally in mammalian cells. For example (but not by way of limitation), a peptide analog may contain D-alanine, naphthylalanine, pyridylalanine, and norleucine. A peptide analog may also have an extension of one or more naturally occurring and/or nonnatural amino acids at its amino terminus and/or its carboxyl terminus. The extension at the amino terminus and/or the carboxyl terminus may include one or more additional copies of the same peptide and/or other bioactive peptides. The extension at the amino terminus and/or the carboxyl terminus may include one or more sites for proteolytic processing in order to make the extended peptide function as a precursor (prodrug) for the bioactive peptide. For example, the PACAP analogs may include cleavage sites at the amino terminus and/or the carboxyl terminus for one or more of the following proteolytic enzymes: trypsin, chymotrypsin, a prohormone convertase (e.g., prohormone convertase 1, 2, 4, 5, or 7), furin, chymase, thrombin, calpain, a cathepsin (e.g., cathepsin A, B, D, G, H, or L), papain, Factor Xa, Factor IXa, Factor XIa, renin, chymosin (rennin), thermolysin, a kallikrein, an elastase, and a matrix metalloproteinase.

As used herein, the term “PACAP analog” refers to human PACAP27 (SEQ ID NO:2), human PACAP38 (SEQ ID NO:1), human VIP (SEQ ID NO:3), lizard PACAP38 (Valiante et al., Brain Res 1127:66-75, 2007), frog PACAP38 (Chartrel et al., Endocrinology 129:3367-3371, 1991), and sand fly maxadilan (Lanzaro et al., Insect Mol Biol 8:267-275, 1999), and peptides or peptidomimetics compounds that are orthologs, paralogs, analogs, fragments, or derivatives of these naturally occurring peptides and that have agonist activity at one or more PACAP/VIP receptors. PACAP analogs of the invention include those having the sequence set forth in SEQ ID NOs: 4-78 (e.g., SEQ ID NOs: 4-14) and polypeptides having at least 80, 85, 90, 95, 97, or 99% or more sequence identity to the sequences of SEQ ID NOs: 4-78 (e.g., SEQ ID NOs: 4-14).

As used herein, the term “peptidomimetic” refers to both hybrid peptide/organic molecules and nonpeptide organic molecules that have critical functional groups in a three-dimensional orientation that is functionally equivalent to the corresponding peptide (Marshall, Tetrahedron 49:3547-3558, 1993). Peptidomimetic compounds that are functionally equivalents to the PACAP analogs of the present invention can be rationally designed by those skilled in the art based on published structure-activity studies (e.g., Igarashi et al., J Pharmacol Exp Ther 301:37-50, 2002; Igarashi et al., J Pharmacol Exp Ther 303:445-460, 2002; Bourgault et al., Peptides 29:919-932, 2008; Bourgault et al., J Med Chem 52:3308-3316, 2009).

The terms “percent identity” and “percent similarity” can be used to compare the amino-acid sequences of two peptides. To determine the percent identity of two amino acid sequences, the sequences are aligned for optimal comparison purposes (e.g., gaps can be introduced in the sequence of a first amino-acid sequence for optimal alignment with a second amino-acid sequence). The amino-acid residues at the corresponding amino-acid positions are then compared. When a position in the first sequence is occupied by the same amino-acid residue at the corresponding position in the second sequence, then the molecules are identical at that position. The percent identity between the two sequences is a function of the number of identical positions shared by the sequences (i.e., % identity=the number of identical overlapping positions/total number of positions×100%). In the most common embodiment, the two amino-acid sequences are the same length. To determine the percent similarity of two amino acid sequences, the sequences are also aligned for optimal comparison purposes. When a position in the first sequence is occupied by either the same amino-acid residue or a “conserved” amino acid at the corresponding position in the second sequence, then the molecules are similar at that position. The percent similarity between the two sequences is a function of the number of corresponding positions in the amino acid sequences at which the amino acids are either identical or the different amino acids are conserved substituents (i.e., % similarity=the number of identical or conserved overlapping positions/total number of positions×100%). A “conservative substitution” is a substitution of one amino acid by another amino acid with a similar side-chain. A conservative substitution almost always results in an analog with similar physical and biological properties. The following is a list of commonly defined classes of “similar” amino acids that occur naturally in mammalian peptides.

Aromatic side-chain: phenylalanine≅tyrosine≅tryptophan≅histidine

Acidic side-chain: aspartic acid≅glutamic acid

Basic side-chain: arginine≅lysine≅histidine

β-Branched side-chain: threonine≅valine≅isoleucine

Nonpolar side-chain: alanine≅valine≅leucine≅proline≅methionine≅phenylalanine≅tryptophan

Uncharged polar side-chain: glycine≅asparagine≅glutamine≅serine≅threonine≅cysteine tyrosine

Changes in the amino acid sequence of PACAP38 during evolution are often conservative substitutions. For example, the only amino acid difference between frog (Xenopus laevis) PACAP38 and human PACAP38 is a substitution of isoleucine for valine, while the only amino acid difference between lung fish (Protopterus dolloi) PACAP38 and human PACAP38 is a substitution of lysine for arginine.

Those skilled in the art will recognize that many amino acids that occur naturally in mammalian cells but do not occur naturally in mammalian peptides and many nonnatural amino acids that do not occur naturally in mammalian cells can be substituted conservatively for one or more of the amino acids that occur naturally in mammalian peptides. For example (but not by way of limitation), hydroxyproline, dehydroproline and N-alkylamino acids could be substituted conservatively for proline, sarcosine, dialkylglycine and α-aminocycloalkane carboxylic acid could be substituted conservatively for glycine, and α-aminobutyric acid, α-aminoisobutyric acid and β-alanine could be substituted conservatively for alanine. “Percent identity” and “percent similarity” are determined after optimal alignment of the two sequences without or without the introduction of one or more gaps in one or both amino-acid sequences. There are many algorithms that are well known to those skilled in the art that can be used to determine the optimal alignment. In the most common embodiment, the two amino-acid sequences are the same length.

As used herein, the term “fragment” in the context of PACAP-like or VIP-like peptides refers to a peptide that has fewer amino acids than the PACAP-like or VIP-like peptide and has at least five amino acids with sequence similarity to the PACAP-like or VIP-like peptide, respectively.

As used herein, the term “derivative” refers to a peptide that has been modified by the covalent attachment of another molecule and/or a functional group to the peptide chain. For example (but not by way of limitation), a derivative of a peptide may be produced by glycosylation, acetylation, pegylation, acylation, alkylation, oxidation, phosphorylation, sulfation, formylation, methylation, demethylation, amidation, gamma-carboxylation, cyclization, lactamization, prenylation, myristoylation, iodination, selenoylation, ribosylation, ubiquitination, or hydroxylation. The derivatized peptide can be a peptide analog. A derivative of a peptide can easily be made by standard techniques known to those of skilled in the art. A derivative of a peptide may possess an identical function(s) to the parent peptide. A derivative of a peptide may also have one or more other functions in addition to the function(s) of the parent peptide. For example (but not by way of limitation), a derivative of a peptide may have a longer half-life than the parent peptide and/or have cytoprotective or cytotoxic properties that are not possessed by the parent peptide.

As used herein, the term “subject” refers to either a non-primate (e.g., a cow, pig, horse, cat, dog, rat, etc.) or a primate (e.g., a monkey or a human being), most preferably a human being. In a specific embodiment, the subject is a farm animal (e.g., a horse, pig, lamb or cow) or a pet (e.g., a dog, cat, rabbit, or monkey). In another embodiment, the subject is an animal other than a farm animal or a pet (e.g., a mouse, rat or guinea pig). In a preferred embodiment, the subject is a normal human being. In another preferred embodiment, the subject is a human that has an untreated or treated cancer.

As used herein, the term “in combination with” refers to the use of more than one therapeutic or cytoprotective agent. The use of the term “in combination with” does not restrict the order in which the therapeutic or cytoprotective agent is administered to a subject. One therapeutic or cytoprotective agent can be administered prior to, concomitantly with, or subsequent to the administration of the other therapeutic or cytoprotective agent. The therapies are administered to a subject in a sequence and within a time interval such that the PACAP analog(s) of the present invention can act together with the other agent to provide a different response from the subject, preferably a greater therapeutic or cytoprotective benefit, than if they were administered otherwise.

As used herein, the term “therapeutic index” refers to the dose of a therapeutic agent that produces an unacceptable toxic effect (TD) in 50% of the patient population divided by the dose of the therapeutic agent that produces the therapeutic effect (ED) in 50% of the patient population, that is,

$$Therapeutic Index = \frac{{TD}_{50}}{{ED}_{50}}$$

All adjunctive therapeutic agents currently approved by the FDA, such as amifostine, dexrazoxane and mesna, increase the TD₅₀ with (presumably) little or no increase in the ED₅₀. PACAP-like peptides are unique. If an appropriate disease is chosen, such as a leukemia or a plasma cell dyscrasia, and an appropriate primary therapeutic is chosen, such as methotrexate or cisplatin, adding a PACAP analog to the treatment regimen can simultaneously increase the TD₅₀ and decrease the ED₅₀.

As used herein, the term “nervous system” refers to the central nervous system (the brain and spinal cord), the sympathetic nervous system, the parasympathetic nervous system, and the enteric nervous system.

As used herein, the term “gastrointestinal tract” refers to the pharynx, esophagus, stomach, small intestine, pancreas, and large intestine.

As used herein, the term “hematological malignancies” refers to cancers of blood cells, bone marrow cells or cells of the lymph nodes, including (but not limited to) leukemias, lymphomas and plasma cell dyscrasias.

As used herein, the phrase “plasma cell dyscrasias” refers to monoclonal neoplasms of the B-lymphocyte lineage, including (but not limited to) multiple myeloma, Waldenström's macroglobulinemia, POEMS syndrome, Seligman's disease, and Franklin's disease.

As used herein, the adjective “hematopoietic” refers to cells (including cancer cells) that are derived from hematopoietic stem cells. The normal cells of the body that are derived from hematopoietic stem cells include (but are not limited to) erythrocytes, granulocytes (basophils, eosinophils and neutrophils), lymphocytes, monocytes (macrophages, microglia, splenocytes, and dendritic cells), and thrombocytes.

As used herein, the term “about” refers to a value that is ±10% of the recited value.

### DETAILED DESCRIPTION OF THE INVENTION

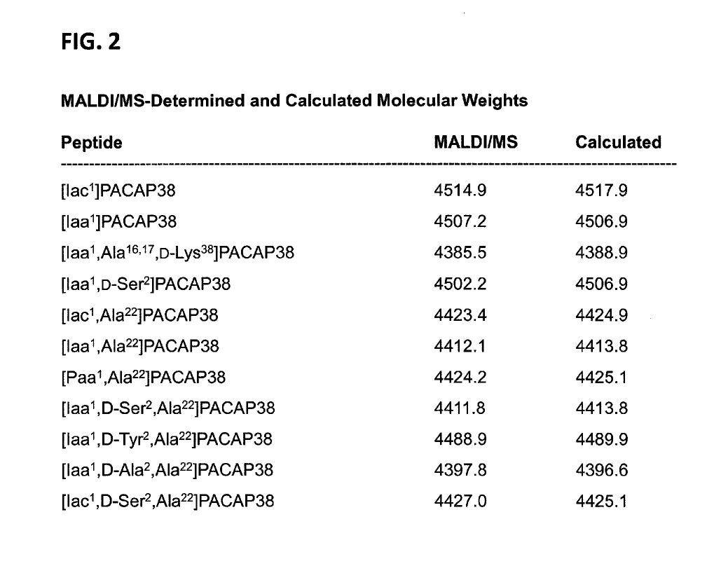

The inventors of the present patent application have discovered that replacing histidine in position 1 of native human PACAP38 with imidazole-4-acrylic acid (Iaa), imidazole-4-acetic acid (Iac), or 3-pyridylacetic acid (Paa) results in three series of PACAP analogs (SEQ ID NOs. 4-14, FIG. 2) with increased half-life in serum and unique pharmacological properties. In particular, both imidazole-4-acetic acid (Iaa) and imidazole-4-acrylic acid (Iac) reduce the affinity of the PACAP analogs for the VPAC1 and VPAC2 receptors, but not for the PAC1 receptor. Thus, the imidazolic acid analog substitutions at position 1 of PACAP38 may be used to make the PACAP analogs specific for PAC1 receptors. Moreover, because imidazole-4-acetic acid (Iaa) and imidazole-4-acrylic acid (Iac) in position 1, as well as substitutions in position 22, reduce VPAC2 binding substantially without affecting PAC1 or VPAC1 binding, these substitutions may be used to make the PACAP analogs specific for both PAC1 and VPAC1 receptors.

Furthermore, substitutions in other positions of the PACAP analogs besides position 1 can be made in order to block proteolysis and/or renal clearance, reduce the cost of synthesis, and/or alter tissue distribution and/or receptor specificity. In particular, substitution at position 17 removes a methionine residue to avoid oxidation. In addition, PACAP is degraded in the circulation by peptidases, such as aminodipeptidases and carboxypeptidases. Removing the amino group at the N-terminus of PACAP analogs should make them resistant to aminopeptidases. The substitution at position 38 makes the PACAP analogs resistant to carboxypeptidases.

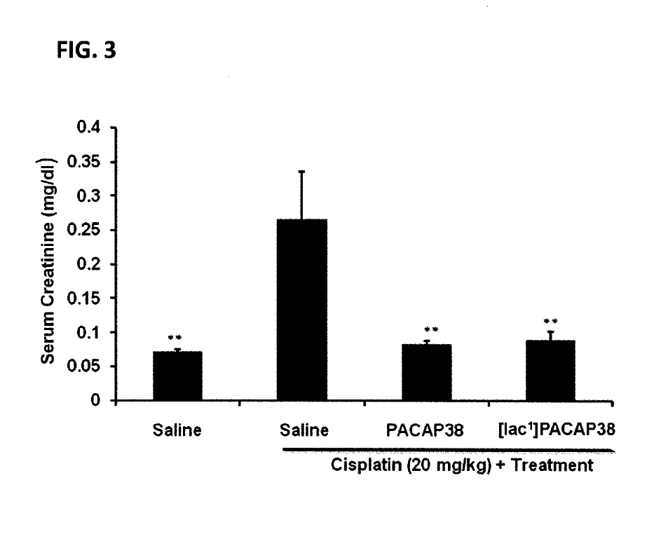

The inventors of the present patent application have discovered that the nephrotoxicity caused by cisplatin in mice can be dramatically reduced by the intraperitoneal injection of one of these PACAP analogs (FIGS. 3-8).

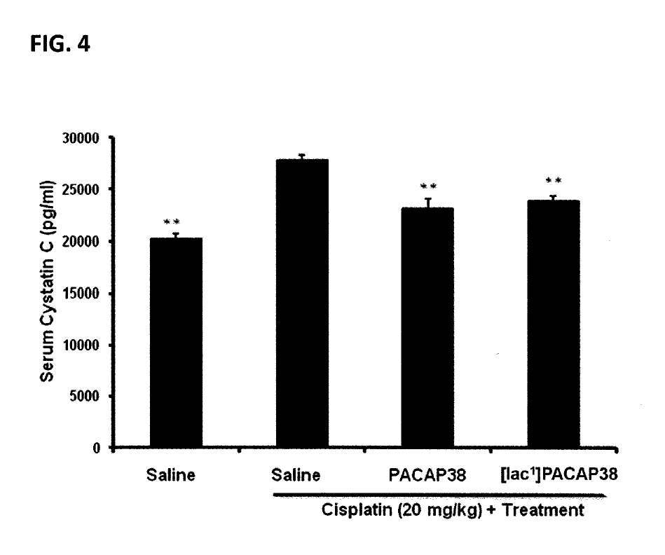

The inventors of the present patent application have discovered that the increases in serum creatinine and cystatin C levels caused by cisplatin can be dramatically reduced by treatment with [Iac1]PACAP38 (FIGS. 3 and 4).

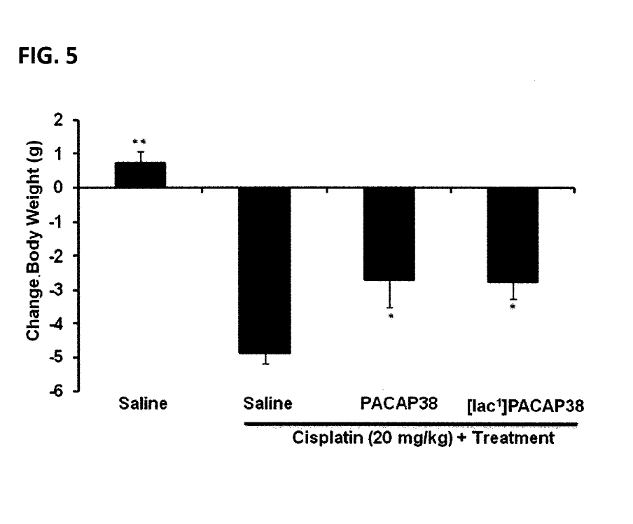

The inventors of the present patent application have discovered that the decrease in body weight caused by cisplatin can be dramatically reversed by treatment with [Iac1]PACAP38 (FIG. 5).

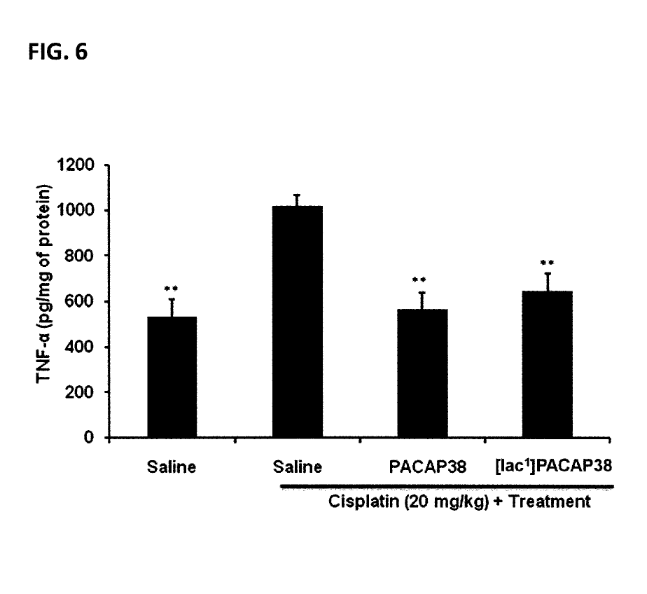

The inventors of the present patent application have discovered that the increase in the levels of tumor necrosis factor-α, interferon-γ and monocyte chemotactic protein 1 in the kidney caused by cisplatin can be dramatically reversed by treatment with [Iac1]PACAP38 (FIGS. 6-8).

The inventors of the present patent application have discovered that these PACAP analogs of the invention can be used as prophylactic/therapeutic agents for a wide range of medical disorders, including (but not limited to) age-related neurodegenerative diseases (such as Alzheimer's disease, Parkinson's disease and amyotrophic lateral sclerosis), injuries to the central nervous system caused by stroke, heart attack and blunt force trauma (such as concussions and spinal cord trauma), Huntington's disease and other CAG codon repeat expansion diseases, retinal diseases (such as ischemia/reperfusion injury, non-infectious uveitis, diabetic retinopathy, macular degeneration, and glaucoma), autoimmune diseases (such as rheumatoid arthritis, Crohn's disease, ulcerative colitis, scleroderma, Sjögren's disease, idiopathic membranous nephropathy, Goodpasture's disease, autoimmune hepatitis, autoimmune myocarditis, myasthenia gravis, multiple sclerosis, Guillain-Barré syndrome, type I diabetes, Hashimoto's thyroiditis, Graves' disease, pemphigus vulgaris, and systemic lupus erythematosus), graft-versus-host disease, keratoconjunctivitis sicca caused by aging, autoimmune diseases, corneal transplantation, or keratorefractive surgery, type II diabetes, sepsis caused by bacteria and/or viruses (including bacterial and viral toxins), acute and chronic cardiovascular diseases (such as myocardial infarction, atherosclerosis, restenosis, and drug-induced cardiomyopathy), acute and chronic renal diseases (such as ischemia/reperfusion injury, nephritis and drug-induced nephrotoxicity), genetic disorders caused by premature in-frame stop codons, acute and chronic pulmonary diseases (such as asthma, chronic obstructive pulmonary disease, cystic fibrosis, and pulmonary arterial hypertension), systemic hypertension, hematological cancers (such as leukemias, lymphomas and plasma cell dyscrasias), DNA damage caused by therapeutic agents and radiation, eating disorders, acute and chronic liver diseases (such as ischemia/reperfusion injury, hepatitis and fatty liver), osteoporosis, pre-eclampsia, cell and solid organ transplantation, cognitive disorders, AIDS dementia complex, and aging of the central nervous system.

The inventors of the present patent application have discovered that the PACAP analogs of the invention coupled to suitable radionuclides can be used in the localization, diagnosis and treatment of disseminated cancers and metastatic tumors, and coupled to small molecule therapeutics can be used as vectors for targeted drug delivery. The inventors of the present patent application have discovered that the PACAP analogs of the invention coupled to suitable imaging agents can also be used in the localization of granulomas in humans or other mammals with various infectious or autoimmune diseases.

### Identification of PACAP Analogs

The present invention provides methods for assaying and screening for PACAP-like activity by incubating the compounds with epithelial cells containing one or more PACAP/VIP receptors, e.g., kidney, lung or liver epithelial cells, and multiple myeloma cells, and then assaying for a reduction in a pathology-causing cell phenotype and inhibition of multiple myeloma cell proliferation, respectively (Li et al., Regul Pept 145:24-32, 2008). For example, a PACAP-like peptide or peptidomimetic should increase the viability of cisplatin-treated kidney epithelial cells and decrease the rate of proliferation of multiple myeloma cells. In addition, the intrinsic activity of any PACAP analog at each of the three PACAP/VIP receptors can be determined in stably transfected cell lines that express only one of these receptors by measuring the intracellular accumulation of cyclic AMP (Tatsuno et al., Brain Res 889:138-148, 2001). Radioligand receptor binding assays can be used to determine the affinity of a compound for each of the PACAP/VIP receptors. However, radioligand receptor binding assays do not differentiate between receptor agonists and receptor antagonists. Therefore, other types of assays well known to those skilled in the art must be used to discriminate between PACAP/VIP receptor agonists and PACAP/VIP receptor antagonists.

The viability of renal, pulmonary, hepatic, and neuronal epithelial cells can be determined by a variety of techniques well known to those skilled in the art, including (but not limited to) quantification of the fragmentation of nuclear DNA or caspase 3 activity, quantification of annexin V binding, counting of apoptotic (pyknotic) cells and counting of Trypan blue-positive cells. In the preferred embodiment, the fragmentation of nuclear DNA or caspase 3 activity is determined.

The cell proliferation of hematopoietic and epithelial cells can be determined by a variety of techniques well known to those skilled in the art, including (but not limited to) quantification of the incorporation of bromodeoxyuridine or [³H]thymidine into nuclear DNA, counting of the number of cells expressing proliferating cell nuclear antigen and counting of mitotic figures. In the preferred embodiment, the incorporation of bromodeoxyuridine or [³H]thymidine into nuclear DNA is determined.

The intracellular accumulation of cyclic AMP in stably transfected cell lines that express only one of these receptors can be determined following stimulation with PACAP analogs by a variety of techniques well known to those skilled in the art, including (but not limited to) a radioimmunoassay or an enzyme-linked immunosorbent assay. The stimulation is stopped by the addition of ice-cold 20% trifluoroacetic acid. The cAMP is extracted from the cells, the extracts are centrifuged, the supernatants are placed into small plastic vials, and the supernatants are lyophilized for assay of the levels of cAMP. In the preferred embodiment, the intracellular levels of cAMP are quantified with an enzyme-linked immunosorbent assay.

### Patient Populations

The present invention provides methods for treating, preventing and managing damage caused by trauma, acute or chronic diseases, or one or more prophylactic/therapeutic agents to one or more major organs of the body, such as nervous system, brain, spinal cord, heart, lung, kidneys, liver, pancreas, gall bladder, gastrointestinal tract, adrenal gland, thymus, spleen, lymph nodes, breast, ovary, testes, cornea, and prostate, preferably nervous system, heart, lung, kidneys, liver, cornea, and gastrointestinal tract, of humans or other mammals by the therapeutic or prophylactic administration of effective amounts of one or more compositions of the present invention, such as the polypeptides of SEQ ID NOs: 4-78 (e.g., SEQ ID NOs: 4-14). In another embodiment, the PACAP analogs of the present invention can be administered in combination with one or more other cytoprotective agents.

The methods and compositions of the present invention consists of the administration of one or more compositions of the invention to subjects with injuries caused by trauma, acute or chronic diseases, or one or more prophylactic/therapeutic agents who have suffered from, are suffering from or are expected to suffer from the side-effects of one or more prophylactic/therapeutic agents. In a preferred embodiment, the subject has been, is being or is expected to be administered one or more cancer chemotherapeutics for a hematological malignancy. In the most preferred embodiment, the hematological malignancy is multiple myeloma.

The subjects may or may not have previously been treated on one or more occasions for trauma injuries, acute or chronic diseases, or the side-effects of one or more prophylactic/therapeutic agents. The subjects may or may not have previously been refractory to one or more prophylactic/therapeutic agents. The methods and compositions of the present invention may be used as an adjuvant for a first line, second line or nonstandard treatment regimen for trauma, acute or chronic diseases, or one or more prophylactic/therapeutic agents. The methods and compositions of the present invention can be used before any trauma, acute or chronic diseases, or the side-effects of one or more prophylactic/therapeutic agents are observed or after the first or later observations of any trauma, acute or chronic diseases, or the side-effects of one or more prophylactic/therapeutic agents.

### Other Therapeutic/Prophylactic Agents

In some embodiments, the present invention provides methods for treating, managing or preventing of injuries to one or more of the major organs of the body of humans or other mammals caused by trauma, acute or chronic diseases, or one or more prophylactic/therapeutic agents by administering one or more compositions of the present invention in combination with one or more other cytoprotective agents. These other cytoprotective agents include (but are not limited to) amifostine, dexrazoxane, mesna, palifermin, apocynin, erythropoietin, N-acetylcysteine, and N-acetylcysteine amide. None of the listed cytoprotective agents stimulate G-protein-coupled receptors and all of these cytoprotective agents have mechanisms of action that are distinct from the presumed cytoprotective mechanisms of action of PACAP-like peptides. Therefore, one or more of these cytoprotective agents can have additive or even synergistic effects when administered in combination with PACAP analogs, e.g., one or more of the PACAP analogs of the invention, such as SEQ ID NOs: 4-78 (e.g., SEQ ID NOs: 4-14).

### Synthesis of the PACAP Analogs of the Invention

Peptides were prepared by modified solid-phase procedures using Fmoc chemistries on a CEM microwave-assisted automatic peptide synthesizer (Matthews, N.C.) followed by trifluoroacetic acid (TFA) resin cleavage. Briefly, a standard Rink amide resin (Advanced CheTech, Louisville, Ky.) was used to yield peptide amides directly after TFA cleavage. Treatment with 20% piperidine in a dimethylformamide solution containing 0.2 M 1-hydroxybenzotriazole (HOBt) acid (2 minutes at 70° C.) was used for Fmoc group removal and amino acid couplings were achieved using a 4 M excess of each protected amino acid and 1 equivalent of the PyClocK reagent (Peptides International, Louisville, Ky.) and 2 equivalents of 0.2 M diisoprpropyethyamine in a dimethylformamide solution at 70° C. accompanied by microwave irradiation except for Fmoc-His(Trt), which was coupled at 50° C. for 15 minutes. Fmoc amino acid side-chain protection groups commonly used were: Asp, Glu, Ser, Thr, and Tyr: tBu; Arg: Pbf; Lys, Orn, Dab, and Dap: Boc; and His: Trt.

Peptide are simultaneously deprotected and cleaved from the resin support by shaking at room temperature for 4 hours with a mixture of TFA containing 1% water and 1% triisopropylsilane. The resin and solution was then poured into a large excess of cold diethylether and the precipitate and resin filtered through a fine glass frit. After washing with ether and allowing to dry, the cleaved peptide was extracted from the resin using dilute acetic acid/water mixtures. The resulting solutions were applied directly to preparative chromatography systems (either 1.5 or 2.5×25 cm columns) containing Vydac C-I8 silica of 300-angstrom pore size (particle size 10 μm). Two fully volatile solvent elution systems have been used successfully for all of these peptides: linear gradients of acetonitrile in 0.1% TFA or acetonitrile in 20% acetic acid (which was excellent for insoluble peptides) at flow rates of about 8-20 ml/min. Fractions containing the desired peptide in acceptable purity (>95%) were identified using analytical high-performance liquid chromatography (HPLC) and MALDI MS and then lyophilized.

A long-chain saturated fatty acid could be covalently linked to the free epsilon-amino group of one of the four Lys residues near the C-terminus of PACAP38 (SEQ ID NO:1), [Iac¹]PACAP38 (SEQ ID NO:4), [Iaa¹]PACAP38 (SEQ ID NO:5), [Iaa¹,Ala^{16,17},D-Lys³]PACAP38 (SEQ ID NO:6), [Iaa¹,D-Ser²]PACAP38 (SEQ ID NO:7), [Iac¹,Ala²²]PACAP38 (SEQ ID NO:8), [Iaa¹,Ala²²]PACAP38 (SEQ ID NO:9), [Paa¹,Ala²]PACAP38 (SEQ ID NO:10), [Iaa¹,D-Ser²,Ala²²]PACAP38 (SEQ ID NO:11), [Iaa¹,D-Tyr²,Ala²²]PACAP38 (SEQ ID NO:12), [Iaa¹,D-Ala²,Ala²²]PACAP38 (SEQ ID NO:13) [Iac¹,D-Ser²,Ala²²]PACAP38 (SEQ ID NO:14), or PACAP38 analogs containing similar free amino group-containing amino acids such as Orn, Dab and Dap near the C-terminus. PACAP27 and PACAP38 have similar affinities for the PAC₁, VPAC₁ and VPAC₂ receptors suggesting that the additional 11 amino acids are not essential for high-affinity receptor binding. The fatty acid attachment will promote high-affinity binding of the conjugate to serum albumin (Kurtzhals et al., J Pharm Sci 85:304-308, 1996), which is by far the most abundant protein in serum, and dramatically reduce the rate of filtration by the kidney. This strategy has been used to make long-acting analogs of GLP-1 (Knudsen et al., J Med Chem 43:1664-1669, 2000), which is a member of the secretin/VIP/PACAP family.

The purity of each purified compound was confirmed by analytical HPLC and MALDI MS.

### Therapeutic or Anticancer Agents Administered with or Coupled to PACAP Analogs of the Invention

The PACAP analogs of the invention (e.g., PACAP analogs having the structure of Formula (I); e.g., PACAP analogs having the sequence of SEQ ID NOs: 4-78 (e.g., SEQ ID NOs: 4-14)) can be administered or formulated with, or coupled to, a therapeutic or anticancer agent. Preferred anticancer agents for administration or formulation with, or coupling to, e.g., PACAP analogs having the structure of Formula (I) (e.g., PACAP analogs having the sequence of SEQ ID NOs: 4-78 (e.g., SEQ ID NOs: 4-14)) include cisplatin, carboplatin, oxaliplatin, bleomycin, mitomycin C, calicheamicins, maytansinoids, auristatins, esperamicins, geldanamycin, doxorubicin, idarubicin, daunorubicin, epirubicin, busulfan, carmustine (BCNU), lomustine (CCNU), semustine, fotemustine, bendamustine, nimustine, thalidomide, lenalidomide, methotrexate, azathioprine, 6-mercaptopurine, fludarabine, 5-azacytidine, pentostatin (2′-deoxycoformycin), cytarabine (cytosine arabinoside), gemcitabine, 5-fluorouracil, hydroxyurea, elesclomol (STA-4783), etoposide, teniposide, amsacrine, mitoxantrone, camptothecin, topotecan, irinotecan, chlorambucil, cyclophosphamide, ifosfamide, melphalan, bortezomib, vincristine, vinblastine, vinorelbine, paclitaxel, and docetaxel. Preferred therapeutic agents for administration or formulation with, or coupling to, e.g., PACAP analogs having the structure of Formula (I) (e.g., PACAP analogs having the sequence of SEQ ID NOs: 4-78 (e.g., SEQ ID NOs: 4-14)) include cyclosporine A, tacrolimus (FK506), sirolimus (rapamycin), everolimus, temsirolimus, zotarolimus, and biolimus.

The PACAP analogs of the invention can be coupled directly to a therapeutic or anticancer agent using known chemical methods. Alternatively the PACAP analogs can be coupled to an anticancer or therapeutic agent via an indirect linkage.

### Diagnostic Agents Coupled to PACAP Analogs of the Invention

PACAP analogs of the invention can be modified or labeled to facilitate diagnostic or therapeutic uses. Detectable labels, such as a radioactive, fluorescent, heavy metal, or other agent may be bound (ionically or covalently) to the PACAP analogs of the invention.

Radioisotopes for radiolabeling the PACAP analogs of the invention can be selected from radioisotopes that emit either beta or gamma radiation. Alternatively, PACAP analogs of the invention can be modified to contain a chelating group. The chelating group can then be modified to contain any of a variety of radioisotopes.

PACAP analogs of the invention can be coupled to a chelating agent to form diagnostic conjugate of the invention. Chelating groups may be used to indirectly couple detectable labels or other molecules to PACAP analogs of the invention. Chelating groups may be used to link radiolabels to the PACAP analogs of the invention.

### Demonstration of the Therapeutic Usefulness

The protocols and compositions of the present invention are preferably tested in vitro, and then in preclinical models in vivo, for the desired therapeutic or prophylactic activity, prior to use in humans. For example, in vitro assays that can be used to determine whether administration of a specific therapeutic protocol is indicated, include in vitro cell culture assays in which an appropriate cell line or a patient's tissue sample is grown in culture, and exposed to or otherwise administered a protocol, and the effect of such protocol upon the tissue sample is observed. For example (but not by way of limitation), rescuing of sensory neurons, renal or pulmonary epithelial cells, hepatocytes, or cardiomyocytes; decreased NFκB activation; decreased survival or proliferation of B- or T-lymphocytes; or decreased production of TNF-α and IL-6. A demonstration of one or more of the aforementioned properties of the exposed cells indicates that the therapeutic agent is effective for treating the condition in the patient. Many assays standard in the art can be used to assess such survival and/or growth of neurons, epithelial cells, hepatocytes, and/or B- or T-lymphocytes. Furthermore, any of the assays known to those skilled in the art can be used to evaluate the prophylactic and/or therapeutic utility of the therapies disclosed herein for treatment, management or prevention of injuries to one or more major organs of the body caused by trauma, diseases or other prophylactic or therapeutic agents.

The injuries to one or more major organs of the body of humans or other mammals caused by trauma, acute or chronic diseases, or one or more prophylactic/therapeutic agents can be monitored in the subjects with commonly used biomarkers. For example (but not by way of limitation), injury to the kidney can be monitored by determining the concentration of protein in the urine, or the concentration of creatinine or urea nitrogen in the bloodstream. Injury to the liver can be monitored by determining the enzyme activity or concentration of alanine aminotransferase in the bloodstream, or the concentration of conjugated bilirubin in the urine. Injury to the heart can be monitored by determining the concentration of troponin I or the MB isoenzyme of creatinine kinase in the bloodstream. Injury to the β-cells of the pancreas can be monitored by determining the activity or concentration of glutamic acid decarboxylase in the bloodstream, and injury to the nervous system can be monitored by determining the activity or concentration of neuron-specific enolase in the bloodstream.

The injuries to one or more major organs of the body of humans or other mammals caused by trauma, acute or chronic diseases, or one or more prophylactic/therapeutic agents can also be monitored in the subjects with commonly used imaging techniques. For example (but not by way of limitation), injury to the heart can be monitored by electrocardiography or serial echocardiography.

The injuries to one or more major organs of the body of humans or other mammals caused by trauma, acute or chronic diseases, or one or more prophylactic/therapeutic agents can also be monitored in the subjects with commonly used functional tests. For example (but not by way of limitation), injury to the kidney can be monitored by determining the glomerular filtration rate with cystatin C or with sodium ¹²⁵I-iothalamate clearance. Injury to the peripheral nerves can be monitored by determining nerve conduction velocities or somatosensory perception. Injury to the heart can be monitored with a variety of exercise tests.

Based on the currently available data, there is a correlation between the reduction in the rate of proliferation of some cancer cells by PACAP analogs and the enhancement of the therapeutic efficacy of anticancer agents by PACAP analogs. Cancer cells can be obtained from biopsy samples from humans and other mammals, cultured in multi-well plates, and the effect of PACAP-like peptides on their rate of proliferation can be quantified in order to determine whether the PACAP analogs will protect the cancer cells against cancer chemotherapeutics or enhance the efficacy of cancer chemotherapeutics.

The definitive diagnosis of multiple myeloma can be made in about 95% of the patients after a bone marrow aspiration or bone marrow biopsy. In the other patients, the bone marrow involvement is probably focal rather than diffuse. The efficacy of the adjunctive treatment with PACAP-like peptides can be determined subjectively by the patient reporting an improvement in symptoms, such as bone pain, fatigue, and overall well-being. The efficacy of the adjunctive treatment with PACAP-like peptides can be determined objectively by a physical examination that shows an improvement in overall appearance and muscle strength, by laboratory tests that show a reduction in anemia (a rise in hemoglobin and hematocrit), serum and urinary levels of the monoclonal paraprotein (Bence-Jones protein), and serum and urinary β-2 microglobulin, and by laboratory tests that show an improvement in kidney function (blood creatinine, urea nitrogen and cystatin C). In a preferred embodiment, serum and urinary levels of the monoclonal free light-chain immunoglobulin (Bence-Jones protein) are monitored with a highly sensitive nephelometric assay during the course of the treatment with the PACAP analogs. Those skilled in the art will recognize, or be able to ascertain using no more than routine searches of the medical literature that there are similar standard methods for selecting appropriate patient populations to study of the effects of the compositions of the present invention on age-related neurodegenerative diseases; injuries to the central nervous system caused by stroke, heart attack and blunt force trauma; Huntington's disease and other CAG codon repeat expansion diseases; retinal diseases; autoimmune diseases; graft-versus-host disease; keratoconjunctivitis sicca; type II diabetes; sepsis; acute and chronic cardiovascular diseases; acute and chronic renal diseases; genetic disorders caused by premature in-frame stop codons, acute and chronic pulmonary diseases; systemic hypertension; hematological cancers; DNA damage caused by therapeutic agents and radiation, eating disorders; acute and chronic liver diseases; osteoporosis; pre-eclampsia; cell and solid organ transplantation; cognitive disorders; AIDS dementia complex; and aging of the central nervous system.

### Pharmaceutical Compositions

The compositions of the present invention include bulk drug compositions useful in the manufacture of pharmaceutical compositions (e.g., impure or non-sterile compositions) and parenteral pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the present invention comprise a prophylactically or therapeutically effective amount of one or more PACAP analogs useful in the method of the invention and a pharmaceutically acceptable carrier. In a further embodiment, the composition of the present invention further comprises an additional therapeutic as discussed above.

In a specific embodiment, the term “pharmaceutically acceptable” means approved by a regulatory agency of the Federal government or listed in the U.S. Pharmacopeia or other generally recognized pharmacopeia for use in animals, and particularly for use in humans. The term “carrier” refers to a diluent, adjuvant (e.g., Freund's adjuvant or, more preferably, MF59C.I adjuvant), excipient, or vehicle with which the therapeutic is administered. The pharmaceutical carriers can be sterile liquids, such as water and oils, including those of petroleum, animal, vegetable or synthetic origin, such as peanut oil, soybean oil, mineral oil, sesame oil. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include (but are not limited to) starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. The composition, if desired, can also contain minor amounts of wetting or emulsifying agents, or pH buffering agents. These compositions can take many forms, including (but not limited to) suspensions, emulsions, tablets, pills, capsules, powders, and sustained-release formulations.

Generally, the ingredients of the compositions of the present invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the present invention can be formulated as neutral or salt forms. Pharmaceutically acceptable salts include (but are not limited to) those formed with anions such as those derived from hydrochloric acid, phosphoric acid, acetic acid, oxalic acid, and tartaric acid, and those formed with cations such as those derived from sodium, potassium, ammonium, calcium, ferric hydroxides, isopropylamine, triethylamine, 2-ethylamino ethanol, histidine, and procaine.

As desired, additives such as a dissolution aid (e.g., sodium salicylate or sodium acetate), a buffer (e.g., sodium citrate or glycerin), an isotonizing agent (e.g., glucose or invert sugar), a stabilizer (e.g., human serum albumin or polyethylene glycol), a preservative (e.g., benzyl alcohol or phenol), or an analgesic (e.g., benzalkonium chloride or procaine hydrochloride) may be added.

There are many delivery methods known to those skilled in the art that can be used to administer the PACAP analogs, or the PACAP analogs in combination with other cytoprotective agents, in order to treat, manage or prevent injuries to one or more of the major organs of the body of humans or other mammals caused by one or more therapeutic or anticancer agents. For example (but not by way of limitation), encapsulation in liposomes, microparticles or microcapsules, secretion from mammalian cells genetically engineered to synthesize one or more PACAP-like peptides, or synthesis by various recombinant viral vectors. The routes of administration of the PACAP analogs of the present invention include (but are not limited to), parenteral (e.g., intradermal, intramuscular, intraperitoneal, intravenous, and subcutaneous), vaginal, rectal, epidural, and mucosal (e.g., intranasal, inhaled, and oral routes). In a specific embodiment, prophylactic or therapeutic agents of the present invention are administered intramuscularly, intravenously, intraosseously, or subcutaneously. The prophylactic or therapeutic agents may be administered by any convenient route or regimen, for example by infusion or a bolus injection, by absorption through epithelial or mucocutaneous linings (e.g., oral mucosa, rectal, topical, including buccal and sublingual, and intestinal mucosa, etc.) and may be administered in combination with other biologically active agents. Administration can be systemic or local.

In a specific embodiment, it may be desirable to administer the prophylactic or therapeutic agents of the present invention locally to the area in need of treatment; this maybe achieved by, for example, but not by way of limitation, local infusion, by injection, or by means of an implant, said implant being of a porous, non-porous, or gelatinous material, including membranes, such as Silastic membranes, or fibers.

In another embodiment, the compositions of this invention can be delivered in a controlled release or sustained release manner. In one embodiment, a pump can be used to achieve controlled or sustained release. In another embodiment, polymeric materials can be used to achieve controlled release or sustained release. Suitable polymers for controlled release or sustained release formulations include (but are not limited to) poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a controlled release or a sustained release formulation is inert, free of leachable impurities, stable on storage, sterile, and biodegradable. In a specific embodiment, a controlled release, or a sustained release device or formulation can be placed in proximity of the prophylactic or therapeutic target, thus reducing the required amount of the PACAP analog to only a fraction of the systemic dose. Many other techniques known to one skilled in the art can be used to produce controlled release or sustained release formulations comprising one or more therapeutic agents of the present invention.

The compositions for administration of the PACAP analogs include (but are not limited to) those suitable for oral, rectal, nasal, topical (including buccal and sublingual), vaginal, or parenteral (including subcutaneous, transcutaneous, intramuscular, intravenous, and intradermal) administration. The formulations may conveniently be presented in unit dosage forms and may be prepared by any methods well known in the art of pharmacy. Thus, the PACAP analogs of the invention and their physiologically acceptable salts and solvates may be formulated for administration by inhalation or insufflation (either through the mouth or the nose), or by oral, parenteral or mucosal (such as buccal, vaginal, rectal, and sublingual) routes. In a preferred embodiment, parenteral administration is used.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium dodecyl sulfate). The tablets may be coated by methods well known in the art. Liquid preparations for oral administration may take the form of, for example, solutions, syrups or suspensions, or they may be presented as a dry product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated to give controlled release or sustained release of the active compound.

For buccal administration, the compositions of the present invention may be conventionally formulated as tablets or lozenges.

For administration by inhalation, the prophylactic or therapeutic agents for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The prophylactic or therapeutic agents may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in a powder form for reconstitution before use with a suitable vehicle, e.g., sterile pyrogen-free water.

In addition to the formulations described previously, the prophylactic or therapeutic agents may also be formulated as a depot preparation. Such long-acting formulations may be administered by implantation (e.g., subcutaneously or intramuscularly) or by intramuscular injection. Thus, for example, the prophylactic or therapeutic agents may be formulated with suitable polymeric or hydrophobic materials (e.g., as an emulsion in an acceptable oil) or ion-exchange resins, or as sparingly soluble derivatives, for example, as a sparingly soluble salt.

Compositions suitable for topical administration to the skin may be presented as ointments, creams, gels, and pastes comprising the compound and a pharmaceutically acceptable carrier. For example (but not by way of limitation), a suitable topical delivery system is a transdermal patch containing the PACAP analog to be administered.

Sublingual tablets can be prepared by using binders (e.g., hydroxypropylcellulose, hydroxypropylmethylcellulose, or polyethylene glycol), disintegrating agents (e.g., starch or carboxymethylcellulose calcium), and/or lubricants (e.g., magnesium stearate or talc).

Suitable formulations for nasal administration wherein the carrier is a solid include a coarse powder having a particle size, for example, in the range 20 to 500 microns (μm). Suitable formulations for nasal administration wherein the carrier is a liquid (e.g., a nasal spray or nasal drops) include aqueous or oily solutions of the active ingredient.

Compositions suitable for parenteral administration include aqueous and non-aqueous sterile injection solutions which may contain anti-oxidants, buffers, bacteriostatic agents, and solutes that make the formulation isotonic with the blood of the intended recipient; and aqueous and non-aqueous sterile suspensions that may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described. It should be understood that in addition to the ingredients specifically mentioned above, the formulations of this invention may include other agents commonly used in the art for the type of formulation in question. For example (but not by way of limitation), those suitable for oral administration may include flavoring agents.

### EXAMPLES

In order to make the uses of the present invention clearer, the following examples are presented. These examples are only for illustrative purposes and should not be interpreted in any way as limitations in the uses of this invention.

### Example 1

### PACAP Analogs

Peptides can have extraordinarily high affinities for their cognate receptors. The major drawback of using native peptides as therapeutics is their short half-life in the circulation after parenteral administration due mainly to rapid proteolysis and rapid filtration by the kidney. Therefore, analogs of PACAP have been made in order to reduce the rates of proteolysis and/or renal clearance. In addition, other changes have been made in the native amino-acid sequences of PACAP27 and PACAP38 in order to reduce the cost of synthesis, and alter tissue distribution and/or receptor specificity.

Eleven peptide analogs of PACAP38 have been made by solid-phase synthesis (SEQ ID NOs 4-14; FIG. 2) using the procedures briefly described above.

The in vivo biological properties of one of these seven PACAP analogs are illustrated below (FIGS. 3-8) and the potential medical applications for these PACAP analogs suggested by these illustrations are briefly outlined.

### Example 2

### Reduction of Cisplatin-Induced Cytotoxicity by PACAP38 and [Iac¹]PACAP38

Cisplatin (cis-diamminedichloridoplatinum(II), Platinol) is the first-in-class platinum-based DNA-crosslinking anticancer therapeutic. It was approved for clinical use by the U.S. FDA in 1978. The other members of this class of “alkylating-like” platinum-based anticancer agents now include (but are not limited to) carboplatin, oxaliplatin and satraplatin. Cisplatin is one of the most widely used cancer chemotherapeutics and is the cornerstone of many multi-drug anticancer regimens. Nephrotoxicity is usually the “dose-limiting” toxicity for the use of cisplatin in cancer chemotherapy, but sensory neuropathies can sometimes limit the doses that can be used to treat some patients.

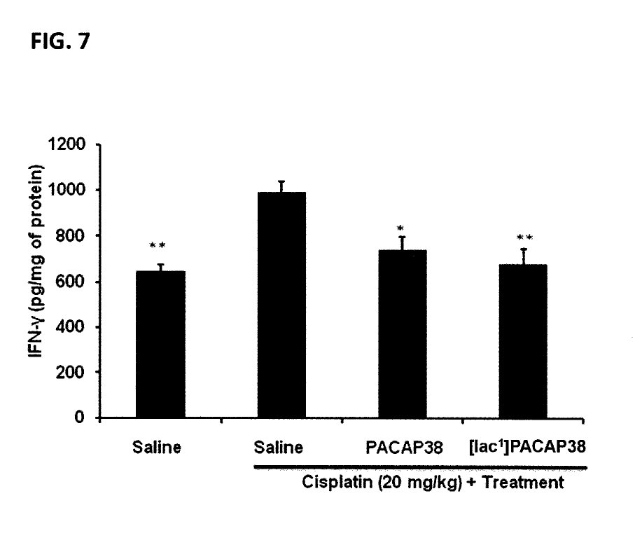

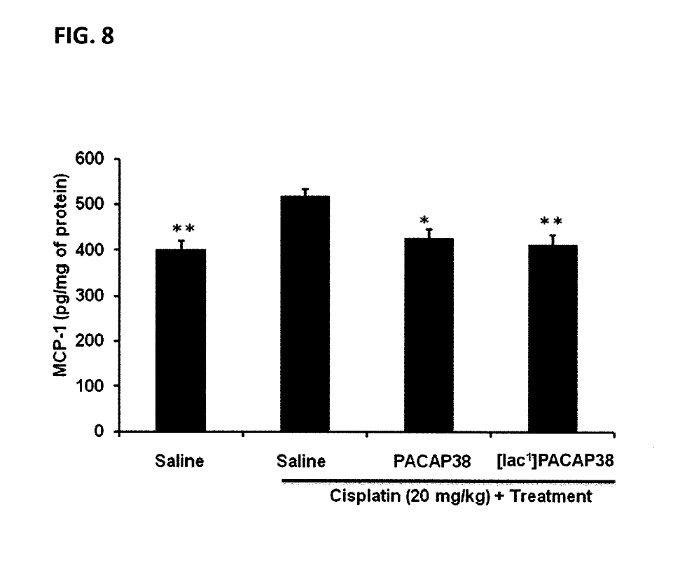

The cytoprotective effect of PACAP38 and [Iac¹]PACAP38 against cisplatin-induced nephrotoxicity was determined in a common in vivo model (Li et al., J Mol Neurosci 43:58-66, 2011). The mice treated with only cisplatin had impaired kidney function as indicated by the significantly increased levels of both creatinine and cystatin in serum compared to the saline-injected control group (FIGS. 3 and 4). The mice treated with only cisplatin also had a significant decline in body weight (FIG. 5). The kidneys from the mice treated with only cisplatin had an inflammatory response as indicated by the significantly elevated levels TNF-α (FIG. 6), IFN-γ (FIG. 7) and MCP-1 (FIG. 8) compared to the saline-injected control group. Treatment of the cisplatin-injected mice with either PACAP38 or [Iac¹]PACAP38 significantly restored kidney function, significantly reversed the weight lost and significantly blunted the inflammatory response (FIGS. 3-8). PACAP38 and [Iac¹]PACAP38 were approximately equipotent as cytoprotectants against cisplatin-induced kidney injury.

These experiments show that PACAP38 and [Iac¹]PACAP38 are potent cytoprotectants against cisplatin-induced damage to the kidney, which is the “dose-limiting” toxicity for cancer chemotherapy with cisplatin. Therefore, pre- and/or post-treatment of subjects undergoing cisplatin-based cancer chemotherapy with therapeutic doses of these PACAP38 analogs should result in a higher maximal tolerable dose of cisplatin, and an increased frequency of partial clinical responses and/or an increased number of complete remissions. We have previously shown that PACAP38 protects the kidney against acute injury due to light-chain immunoglobulin overload, gentamicin, streptozotocin, doxorubicin, and radiocontrast agents (Li et al., Regul Pept 145:24-32, 2008; Maderdrut et al., VIP, PACAP and Related Peptides [Ninth International Symposium], Kagoshima, 2009; Batuman, et al., PCT/US2012/065586, 2012). Therefore, these PACAP38 analogs should also protect the kidney against a similarly broad range of potential nephrotoxins.

### Example 3

### Changes in Arterial Blood Pressure by PACAP38 Analogs

PACAP38 is a directly-acting arterial vasodilator and an indirectly-acting arterial vasoconstrictor. The rapid vasodilator response to intravenous administration of PACAP38 is due to stimulation of PAC₁, VPAC₁ and VPAC₂ receptors in the blood vessels, while the slightly delayed vasoconstrictor response is mainly due to stimulation of PAC₁ receptors in the adrenal medulla and the subsequent release of adrenal catecholamines. Therefore, PACAP38 analogs with different patterns of binding to the three PACAP/VIP receptors would produce different patterns of arterial blood pressure responses following systemic administration. The critical parameter for picking a PACAP38 analog for treating a specific disease is not potency but therapeutic index.

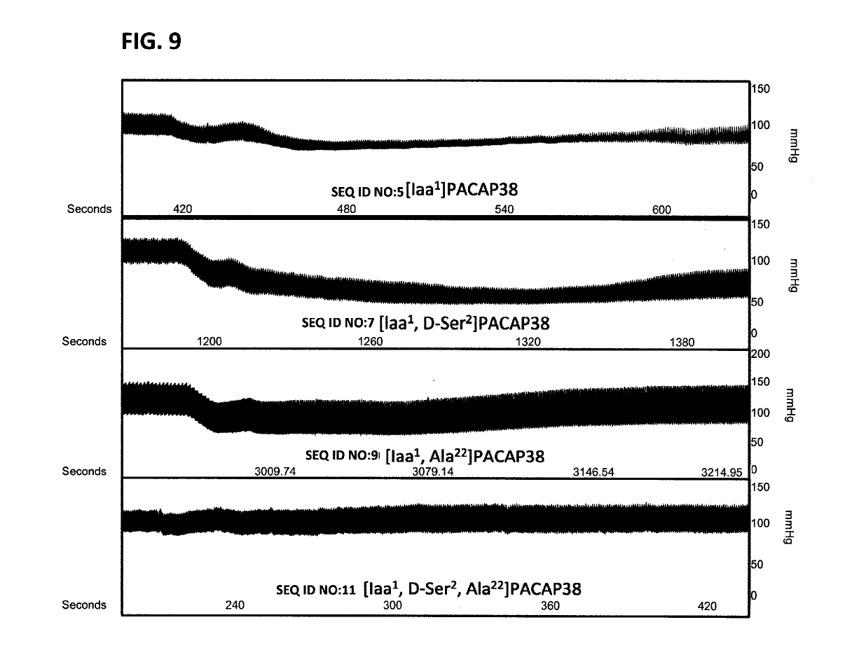

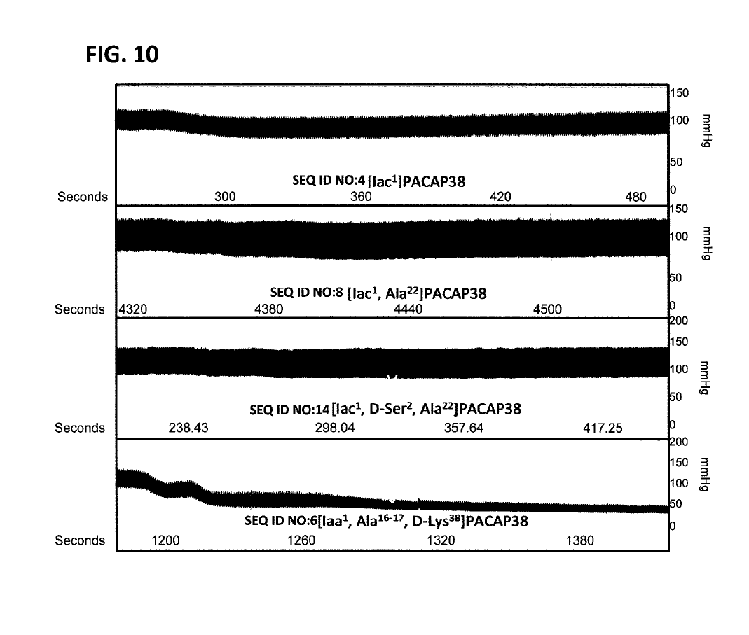

Substitution of the amino acid histidine in position 1 of PACAP38 by either imidazole acetic acid or imidazole acrylic acid resulting in PACAP38 analogs with significant biological activity (FIGS. 3-10). However, the pattern of the arterial blood pressure response following intravenous injection of [Iaa¹]PACAP38 (FIG. 10) was very different from the pattern of the arterial blood pressure response following intravenous injection of [Iac¹]PACAP38 (FIG. 9). The later response pattern was more like the response pattern seen after the intravenous injection of PACAP38. This difference in the pattern of the arterial blood pressure response indicates clearly that [Iaa¹]PACAP38 and [Iac¹]PACAP38 stimulate the three PACAP/VIP receptors in different patterns. The pattern of the arterial blood pressure response following intravenous injection of [Iaa¹,Ala^{16,17}, D-Lys38]PACAP38 (FIG. 10) was similar to the pattern of the arterial blood pressure response following intravenous injection of [Iaa¹]PACAP38 (FIG. 9). Both peptide analogs produced a large decrease in both arterial blood pressure and pulse pressure. However, the duration of the arterial blood pressure response following intravenous injection of [Iaa¹,Ala^{16,17},D-Lys38]PACAP38 was much longer. These observations indicate that the substitution of L-Lys in position 38 of PACAP38 by D-Lys results in significant resistance to carboxypeptidases without a major change in the pattern of stimulation of the three PACAP/VIP receptors. These observations also indicate that substitution of the easily oxidized Met in position 17 of PACAP38 by Ala does not result in a major change in the pattern of stimulation of the three PACAP/VIP receptors. A person with ordinary skill in the art would recognize that L-Lys in position 38 of the other ten PACAP38 analogs (SEQ ID NOs: 4, 5 and 7-14) could be replaced by D-Lys in order to make them resistant to carboxypeptidases without changing their pattern of stimulation of the three PACAP/VIP receptors. A person with ordinary skill in the art would also recognize that Met in position 17 of the other ten PACAP38 analogs (SEQ ID NOs: 4, 5 and 7-14) could be replaced by Ala, Leu, Nle, or Nva in order to remove the easily oxidized Met without changing their pattern of stimulation of the three PACAP/VIP receptors.

PACAP38 analogs with patterns of stimulation of the three PACAP/VIP receptors that differ significantly from the pattern elicited by PACAP38 could have higher therapeutic indices for some disorders than PACAP38. Intravenous injection of [Iaa¹,D-Ser²,Ala²²]PACAP38 increased heart rate from 410 bpm to 457 bpm and slightly increased the arterial blood pressure. This is the expected physiological signature of a highly selective PAC₁ receptor agonist. The arterial blood pressure patterns elicited by the eight PACAP38 analogs (SEQ ID NOs: 4-9, 11 and 14) tested (FIGS. 9 and 10) suggest that [Iac¹,Ala²²]PACAP38 could serve as a building block for developing highly selective proteolysis-resistant agonists for the PAC₁ and VPAC₁ receptors. The arterial blood pressure patterns elicited by the eight PACAP38 analogs (SEQ ID NOs: 4-9, 11 and 14) tested (FIGS. 9 and 10) also suggest that [Iac¹,D-Ser²]PACAP38 could serve as a building block for developing highly selective proteolysis-resistant agonists for the PAC₁ and VPAC₂ receptors.

The above examples show that these PACAP analogs of the invention should be efficacious monotherapeutics and/or adjunctive therapeutics for an extraordinarily wide range of major medical disorders. The above examples also show that these PACAP38 analogs could be used as either monotherapeutics and/or adjunctive therapeutics for both lymphoid and myeloid hematopoietic cancers, for acute and chronic drug-induced nephropathies and cardiomyopathies, and for injuries caused by blunt force trauma, transient arterial stenosis, hemorrhagic shock, severe sepsis, solid organ transplantation, and the side-effects of some common surgical procedures. In addition, these PACAP analogs should be efficacious monotherapeutics and/or adjunctive therapeutics for the extraordinarily wide range of other major medical disorders already shown for native PACAP27, native PACAP38 and native VIP (see SUMMARY OF THE INVENTION).

### EQUIVALENTS

Those skilled in the art will recognize, or be able to ascertain using no more than routine experimentation, many equivalents to the specific embodiments of the present invention described herein. Such equivalents are intended to be encompassed by the following claims.

All publications, patents and patent applications mentioned in this specification are herein incorporated by reference into the specification to the same extent as if each individual publication, patent or patent application was specifically indicated to be incorporated herein by reference.

What is claimed is:

1. A pituitary adenylate cyclase-activating polypeptide (PACAP) analog having formula (I), or a pharmaceutically acceptable salt thereof:
A¹-A²-A³-A⁴-A⁵-A⁶-A⁷-A⁸-A⁹-A¹⁰-A¹¹-A¹²-A¹³-A¹⁴-
A¹⁵-A¹⁶-A¹⁷-A¹⁸-A¹⁹-A²⁰-A²¹-A²²-A²³-A²⁴-A²⁵-A²⁶-
A²⁷-A²⁸-A³⁰-A³¹-A³²-A³³-A³⁴-A³⁵-A³⁶-A³⁷-A³⁸-R¹,
wherein:
A¹ is Iaa, Iac, Ica, or Paa;
A² is Ser, D-Ser, hSer, N-Me-Ser, Thr, D-Thr, D-Tyr, Ala, D-Ala, Ile, D-Ile, Pro, Hyp, Abu, Aib, Acb, Ach, Acpe, or Acpr;
A³ is Asp, D-Asp, Glu, D-Glu, Asn, D-Asn, or N-Me-Asp;
A⁴ is Gly, Sar, Ala, D-Ala, β-Ala, Gaba, Abu, Aib, Acb, Ach, Acpe, or Acpr;
A⁵ is Ile, Leu, Nle, Val, Nva, Aib, Acb, Ach, Acpe, or Acpr;
A⁶ is Phe, Tyr, Pse, Trp, Cha, Bip, Pal, or Nal;
A⁷ is Thr, Ser, hSer, Val, Nva, Ala, or Aib;
A⁸ is Asp, Asn, or Glu;
A⁹ is Ser, hSer, Thr, Asn, Asp, Ala, Abu, Aib, Acb, Ach, Acpe, or Acpr;
A¹⁰ is Tyr, Phe, Pse, Dopa, Cha, Pal, Nal, Trp, Ala, or Aib;
A¹¹ is Ser, hSer, Thr, Ala, Abu, Aib, Acb, Ach, Acpe, or Acpr;
A¹² is Arg, Lys, Dab, Dap, or Orn;
A¹³ is Tyr, Phe, Pse, Dopa, Cha, Pal, Nal, or Trp;
A¹⁴ is Arg, Lys, Dab, Orn, Asn, or Gln;
A¹⁵ is Lys, Ala, Dab, Dap, Orn, Abu, Aib, Acb, Ach, Acpe, Arg, or Acpr;
A¹⁶ is Gln, Glu, Asn, Asp, Ala, Aib, Acb, Ach, Acpe, or Acpr;
A¹⁷ is Met, Nle, Nva, Leu, Ile, Ala, Abu, Aib, Acb, Ach, Acpe, or Acpr;
A¹⁸ is Ala, Abu, Aib, Acb, Ach, Acpe, or Acpr;
A¹⁹ is Val, Nva, Ser, Leu, Thr, Ala, Aib, Acb, Ach, Acpe, or Acpr;
A²⁰ is Lys, Ala, Dab, Dap, Orn, Abu, Aib, Acb, Ach, Acpe, Arg, or Acpr;
A²¹ is Lys, Ala, Dab, Dap, Orn, Abu, Aib, Acb, Ach, Acpe, Arg, or Acpr;
A²² is Tyr, Phe, Pse, Dopa, Cha, Pal, Nal, Trp, Ala, Abu, Aib, Acb, Ach, Acpe, or Acpr;
A²³ is Leu, Nle, Ile, Val, Nva, Aib, Acb, Ach, Acpe, or Acpr;
A²⁴ is Ala, Asn, Abu, Aib, Acb, Ach, Acpe, or Acpr;
A²⁵ is Ala, Val, Leu, Met, Nle, Ile, Ser, hSer, Thr, Abu, Aib, Acb, Ach, Acpe, Acpr, or is omitted;
A²⁶ is Val, Nva, Leu, Met, Nle, Ile, Ala, Abu, Aib, Acb, Ach, Acpe, Acpr, or is omitted;
A²⁷ is Leu, D-Leu, Met, D-Met, Nle, Ile, D-He, Val, D-Val, Gaba, Ala, D-Ala, Abu, Aib, Acb, Ach, Acpe, Acpr, or is omitted;
A²⁸ is Gly, Sar, Ala, D-Ala, β-Ala, Gaba, Asn, D-Asn, Gln, D-Gln, Asp, D-Asp, Abu, Aib, Acb, Ach, Acpe, Acpr, or is omitted;
A²⁹ is Lys, D-Lys, Arg, D-Arg, Dab, D-Dab, Dap, D-Dap, Orn, D-Orn, or is omitted;
A³⁰ is Arg, D-Arg, Lys, D-Lys, Dab, D-Dab, Dap, D-Dap, Orn, D-Orn, or is omitted;
A³¹ is Tyr, D-Tyr, Phe, D-Phe, Pse, D-Pse, Dopa, D-Dopa, Trp, D-Trp, Cha, Pal, Nal, or is omitted;
A³² is Lys, D-Lys, Arg, D-Arg, Dab, D-Dab, Dap, D-Dap, Orn, D-Orn, or is omitted;
A³³ is Gln, D-Gln, Glu, D-Glu, Asn, D-Asn, Asp; D-Asp, Abu, Aib, Acb, Ach, Acpe, Acpr, or is omitted;
A³⁴ is Arg, D-Arg, Lys, D-Lys, Dab, D-Dab, Dap, D-Dap, Orn, D-Orn, or is omitted;
A³⁵ is Val, D-Val, Nva, Ser, D-Ser, Thr; D-Thr, Abu, Aib, Acb, Ach, Acpe, Acpr, or is omitted;
A³⁶ is Lys, D-Lys, Arg, D-Arg, Dab, D-Dab, Dap, D-Dap, Orn, D-Orn, or is omitted;
A³⁷ is Asn, D-Asn, Gln, D-Gln, Asp, D-Asp, Ala, D-Ala, Aib, Acb, Ach, Acpe, Acpr, or is omitted;
A³⁸ is Lys, D-Lys, Arg, D-Arg, Dab, D-Dab, Dap, D-Dap, Orn, D-Orn, or is omitted;
R¹ is independently selected from OH, NH₂, (C₁-C₁₈)alkoxyl, and NH(C₁-C₁₈)alkyl, or is omitted.

2. The PACAP analog of claim 1, wherein the compound is selected from the following, or pharmaceutically acceptable salts thereof:
(SEQ ID NO: 4)
Iac Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 5)
Iaa Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 6)
Iaa Ser Asp Gly Ile Phe Thr Asp Ser Tyr Ser Arg
Tyr Arg Lys Ala Ala Ala Val Lys Lys Tyr Leu Ala
Ala Val Leu Gly Lys Arg Tyr Lys Gln Arg Val Lys
Asn D-Lys-NH₂;
(SEQ ID NO: 7)
Iaa D-Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 8)
Iac Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂,
(SEQ ID NO: 9)
Iaa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 10)
Paa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 11)
Iaa D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 12)
Iaa D-Tyr Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 13)
Iaa D-Ala Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 14)
Iac D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂.

3. A PACAP analog having at least 90% sequence identity to a sequence selected from SEQ ID NOs: 4-78, wherein said PACAP analog comprises an imidazole-4-acetic acid (Iaa), an imidazole-4-acrylic acid (Iac), an imidazole-4-carboxylic acid (Ica), or 3-pyridylacetic acid (Paa) at position 1.

4. The PACAP analog of claim 3, wherein the amino acid residue at position 2 is selected from Ser, D-Ser, hSer, N-Me-Ser, Thr, D-Thr, D-Tyr, Ala, D-Ala, Ile, D-He, Pro, Hyp, Abu, Aib, Acb, Ach, Acpe, or Acpr.

5. The PACAP analog of any one of claims 3 and 4, wherein the amino acid residue at position 16 is selected from Gln, Glu, Asn, Asp, Ala, Aib, Acb, Ach, Acpe, and Acpr.

6. The PACAP analog of any one of claims 3 to 5, wherein the amino acid residue at position 17 is selected from Met, Nle, Leu, Ile, Ala, Abu, Aib, Acb, Ach, Acpe, and Acpr.

7. The PACAP analog of any one of claims 3 to 6, wherein the amino acid residue at position 22 is selected from Tyr, Phe, Pse, Dopa, Cha, Pal, Nal, Trp, Ala, Abu, Aib, Acb, Ach, Acpe, and Acpr.

8. The PACAP analog of any one of claims 3 to 7, wherein the amino acid residue at position 38 is selected from Lys, D-Lys, Arg, D-Arg, Dab, D-Dab, Dap, D-Dap, Orn, and D-Orn, or is omitted.

9. The PACAP analog of any one of claims 3 to 8, wherein said PACAP analog has at least 95% sequence identity to a sequence selected from SEQ ID NOs: 4-78, preferably wherein said PACAP analog has at least 95% sequence identity to a sequence selected from SEQ ID NOs: 4-14.

10. The PACAP analog of any one of claims 3 to 9, wherein said PACAP analog has at least 99% sequence identity to a sequence selected from SEQ ID NOs: 4-78, preferably wherein said PACAP analog has at least 95% sequence identity to a sequence selected from SEQ ID NOs: 4-14.

11. The PACAP analog of any one of claims 3 to 10, further comprising a pharmaceutically acceptable carrier.

12. The PACAP analog of any one of claims 1 to 11, wherein said PACAP analog is conjugated to one or more radionuclides or small molecules.

13. The PACAP analog of claim 12, wherein said radionuclide is ¹¹C, ¹³N, ¹⁵O, ¹⁸F, ⁵²Fe, ⁵⁵Co, ⁶¹Cu, ⁶²Cu, ⁶⁴Cu, ⁶⁷Cu, ⁶⁷Ga, ⁶⁸Ga, ⁶²Zn, ⁶³Zn, ⁷⁰As, ⁷¹As, ⁷⁴As, ⁷⁶Br, ⁷⁹Br, ⁸²Rb, ⁸⁶Y, ⁸⁹Zr, ¹¹⁰In, ¹¹¹In, ¹²⁰I, ¹²³I, ¹²⁴I, ¹²⁵I, ¹³¹I, ¹²²Xe, ¹⁷⁵Lu, ¹⁵⁴Gd, ¹⁵⁵Gd, ¹⁵⁶Gd, ¹⁵⁷Gd, ¹⁵⁸Gd, ^{94m}Tc, ⁹⁴Tc, or ^{99m}Tc.

14. The PACAP analog of claim 12, wherein said small molecule is a therapeutic or anticancer agent.

15. The PACAP analog of claim 14, wherein said therapeutic or anticancer agent is cisplatin, carboplatin, oxaliplatin, bleomycin, mitomycin C, calicheamicins, maytansinoids, auristatins, esperamicins, geldanamycin, doxorubicin, idarubicin, daunorubicin, epirubicin, busulfan, carmustine (BCNU), lomustine (CCNU), semustine, fotemustine, bendamustine, nimustine, thalidomide, lenalidomide, methotrexate, azathioprine, 6-mercaptopurine, fludarabine, 5-azacytidine, pentostatin (2′-deoxycoformycin), cytarabine (cytosine arabinoside), gemcitabine, 5-fluorouracil, hydroxyurea, elesclomol, etoposide, teniposide, amsacrine, mitoxantrone, camptothecin, topotecan, irinotecan, chlorambucil, cyclophosphamide, ifosfamide, melphalan, bortezomib, vincristine, vinblastine, vinorelbine, paclitaxel, docetaxel, iobitridol, iodipamide, iodixanol, iohexol, iomeprol, iopamidol, iopentol, iopromide, iotrolan, ioversol, ioxilan, iothalamate, ioxithalamate, ioxaglate, metrizamide, acetrizoate, metrizoate, diatrizoate, cyclosporine A, tacrolimus (FK506), sirolimus (rapamycin), everolimus, temsirolimus, zotarolimus, or biolimus.

16. A method for treating, managing, or preventing a disease selected from an age-related neurodegenerative disease, a central nervous system disorder, Huntington's disease or other CAG codon repeat expansion disease, a retinal disease, an autoimmune disease, graft-versus-host disease, keratoconjunctivitis sicca caused by aging, autoimmune diseases or keratorefractive surgery, type II diabetes, sepsis caused by a bacteria and/or a virus, an acute or chronic cardiovascular disease, an acute or chronic renal disease, a genetic disorder caused by a premature in-frame stop codon, an acute or chronic pulmonary disease, systemic hypertension, a hematological cancer, a granuloma, an eating disorder, an acute or chronic liver disease, osteoporosis, pre-eclampsia, cell and solid organ transplantation, a cognitive disorder, acquired immunodeficiency syndrome (AIDS) dementia complex, and aging of the central nervous system comprising administering to a subject in need thereof an effective amount of one or more PACAP analogs of any one of claims 1 to 15 or a pharmaceutically acceptable salt thereof.

17. The method of claim 16, wherein:
i) said age-related neurodegenerative disease is selected from Alzheimer's disease, Parkinson's disease and amyotrophic lateral sclerosis;
ii) said central nervous system disorder is caused by stroke, heart attack or blunt force trauma, wherein preferably said blunt force trauma is a concussion or spinal cord trauma;
iii) said retinal disease is ischemia/reperfusion injury, non-infectious uveitis, diabetic retinopathy, macular degeneration, or glaucoma,
iv) said autoimmune disease is rheumatoid arthritis, Crohn's disease, ulcerative colitis, scleroderma, Sjögren's disease, idiopathic membranous nephropathy, Goodpasture's disease, autoimmune hepatitis, autoimmune myocarditis, myasthenia gravis, multiple sclerosis, Guillain-Barré syndrome, type I diabetes, Hashimoto's thyroiditis, Graves' disease, pemphigus vulgaris, or systemic lupus erythematosus;
v) said sepsis is caused by a bacteria or a virus;
vi) said acute or chronic cardiovascular disease is myocardial infarction, atherosclerosis, restenosis, or a drug-induced cardiomyopathy;
vii) said acute or chronic renal disease is ischemia/reperfusion injury, nephritis, or drug-induced nephrotoxicity;
viii) said acute or chronic pulmonary disease is asthma, chronic obstructive pulmonary disease, cystic fibrosis, or pulmonary arterial hypertension;
ix) said hematological cancer is a lymphoid or myeloid hematopoietic cancer, wherein preferably said lymphoid or myeloid hematopoietic cancer is a leukemia, a lymphoma, or a plasma cell dyscrasia;
x) said acute or chronic liver disease is ischemia/reperfusion injury, hepatitis, or fatty liver;
xi) said genetic disorder caused by a premature in-frame stop codon is cystic fibrosis, Duchenne muscular dystrophy, Krabbe's disease (globoid cell leukodystrophy), Hurler's syndrome, retinitis pigmentosa, ataxia telangiectasia, nephropathic cystinosis, or polycystic kidney disease; or
xii) said keratoconjunctivitis sicca is caused by aging, an autoimmune disease or keratorefractive surgery.

18. The method of claim 16 or 17, wherein said subject has an injury to one or more major organs of the body due to treatment with a therapeutic or anticancer agent other than said PACAP analog, trauma, or acute or chronic disease.

19. The method of claim 18, wherein said one or more major organs of body comprise nervous system, brain, spinal cord, heart, lung, kidneys, liver, pancreas, gall bladder, gastrointestinal tract, adrenal gland, thymus, spleen, lymph nodes, breast, ovary, testes, cornea, and prostate, preferably wherein one or more major organs of the body comprise nervous system, heart, lung, kidneys, liver, cornea, and gastrointestinal tract.

20. The method of any one of claims 16 to 19, wherein the PACAP analog of any one of claims 1 to 15, or pharmaceutically acceptable salts thereof, binds to one or more PACAP/VIP receptors and/or reduces one or more injuries to one or more major organs of the body of said subject due to treatment with a therapeutic or anticancer agent other than said PACAP analog, trauma, or acute or chronic disease.

21. The method of any one of claims 16 to 20, wherein said disease is a hematological cancer.

22. The method of any one of claims 16 to 20, wherein said disease is an autoimmune disease.

23. The method of claim 21 or 22, wherein said subject is resistant to treatment with a glucocorticoid.

24. The method of claim 23, wherein said glucocorticoid is dexamethasone, prednisolone, methylprednisolone, or prednisone.

25. The method of claim 21, 23, or 24, wherein said hematological cancer is multiple myeloma.

26. The method of claims 21 to 25, wherein said PACAP analog has the sequence of any one of SEQ ID NOs: 4-78.

27. The method of any one of claims 21 and 23 to 25, wherein said PACAP analog has the sequence of any one of SEQ ID NO: 4-14.

28. The method of claim 21 or 22, wherein said administration of said PACAP analog of any one of claims 1 to 15 in said subject replaces the corticosteroid (prednisone or dexamethasone) using the COP (cyclophosphamide, Oncovin [vincristine] and prednisone) or VAD (vincristine, Adriamycin [doxorubicin] and dexamethasone) regimen.

29. The method of any one of claims 16 to 28, wherein the PACAP analog or a pharmaceutically acceptable salt thereof, linked to a polyethylene glycol polymer with a molecular weight from about 4 kilodaltons to about 40 kilodaltons.

30. The method of any one of claims 16 to 28, wherein the PACAP analog is the unamidated (free acid) form and/or is flanked by amino-acid consensus sequences for one or more proteolytic enzymes.

31. The method of any one of claims 16 to 28, wherein the PACAP analog is a peptidomimetic analog.

32. The method of any one of claims 16 to 31, wherein the PACAP analog of any one of claims 1 to 15 is administered at a dosage that produces a concentration of 10⁻¹⁴ M to 10⁻⁶ M in the blood of the subject.

33. The method of any one of claims 16 to 32, wherein the PACAP analog is administered by intravenous infusion at a rate of about 1 pmol/kg body weight/hour to about 20 pmol/kg body weight/hour.

34. The method of claim 33, wherein the administration by intravenous infusion is for about 1-12 hours.

35. The method of claim 18 or 19, wherein the injuries to one or more major organs of the body are due to treatment with one or more of cisplatin, carboplatin, oxaliplatin, bleomycin, mitomycin C, calicheamicins, maytansinoids, auristatins, esperamicins, geldanamycin, doxorubicin, idarubicin, daunorubicin, epirubicin, busulfan, carmustine (BCNU), lomustine (CCNU), semustine, fotemustine, bendamustine, nimustine, thalidomide, lenalidomide, methotrexate, azathioprine, 6-mercaptopurine, fludarabine, 5-azacytidine, pentostatin (2′-deoxycoformycin), cytarabine (cytosine arabinoside), gemcitabine, 5-fluorouracil, hydroxyurea, elesclomol, etoposide, teniposide, amsacrine, mitoxantrone, camptothecin, topotecan, irinotecan, chlorambucil, cyclophosphamide, ifosfamide, melphalan, bortezomib, vincristine, vinblastine, vinorelbine, paclitaxel, docetaxel, G418, gentamicin, streptomycin, kanamycin, tobramycin, amikacin, arbekacin, dibekacin, neomycin, netilmicin, paromomycin, bekanamycin, hygromycin B, apramycin, sisomicin, isepamicin, astromicin, verdamicin, amphotericin B, rifampicin, pentamidine, iobitridol, iodipamide, iodixanol, iohexol, iomeprol, iopamidol, iopentol, iopromide, iotrolan, ioversol, ioxilan, iothalamate, ioxithalamate, ioxaglate, metrizamide, acetrizoate, metrizoate, diatrizoate, cyclosporine A, tacrolimus (FK506), sirolimus (rapamycin), everolimus, temsirolimus, zotarolimus, or biolimus.

36. The method of claim 18 or 19, wherein the injury is to a kidney of said subject due to treatment with one or more of cisplatin, carboplatin, carmustine, lomustine, semustine, fotemustine, ifosfamide, methotrexate, pentostatin, 5-azacytidine, doxorubicin, daunorubicin, hydroxyurea, mitomycin C, G418, gentamicin, streptomycin, kanamycin, tobramycin, amikacin, arbekacin, dibekacin, neomycin, netilmicin, paromomycin, bekanamycin, hygromycin B, apramycin, sisomicin, isepamicin, astromicin, verdamicin, amphotericin B, rifampicin, pentamidine, iobitridol, iodipamide, iodixanol, iohexol, iomeprol, iopamidol, iopentol, iopromide, iotrolan, ioversol, ioxilan, iothalamate, ioxithalamate, ioxaglate, metrizamide, acetrizoate, metrizoate, diatrizoate, mitoxantrone, cyclosporine A, tacrolimus (FK506), sirolimus (rapamycin), everolimus, temsirolimus, zotarolimus, or biolimus.

37. The method of any one of claims 16 to 36, wherein the PACAP analog is injected intraperitoneally one or more times per day.

38. The method of any one of claims 16 to 36, wherein the PACAP analog is injected subcutaneously one or more times per week.

39. The method of any one of claims 16 to 36, wherein the PACAP analog is injected intramuscularly one or more times per week.

40. The method of any one of claims 11 to 36, wherein the PACAP analog is administered intranasally one or more times per day.

41. The method of any one of claims 16 to 36, wherein the PACAP analog is administered as an aerosol one or more times per day.

42. The method of any one of claims 16 to 36, wherein the PACAP analog is administered orally in a time-dependent or pH-dependent formulation one or more times per day.

43. The method of any one of claims 16 to 36, wherein the PACAP analog is administered as a controlled release or a sustained release formulation.

44. The method of any one of claims 16 to 36, wherein the PACAP analog is administered after encapsulation in liposomes or microparticles.

45. The method of any one of claims 16 to 36, wherein the PACAP analog is administered transcutaneously after encapsulation in dendrimers.

46. The method of any one of claims 16 to 36, wherein the PACAP analog is used to coat a metallic or a biodegradable stent.

47. The method of any one of claims 16 to 46, wherein the PACAP analog is administered in combination with one or more other cytoprotective adjuvants.

48. The method of claim 47, wherein said cytoprotective adjuvant is amifostine, dexrazoxane, mesna, palifermin, apocynin, erythropoietin, N-acetylcysteine, or N-acetylcysteine amide.

49. The method of claim 18 or 19, wherein the injuries to one or more major organs of the body are due to treatment with an unconjugated therapeutic or anticancer agent, a therapeutic or anticancer agent conjugated to a monoclonal antibody or a bioactive peptide, or an unconjugated bioactive peptide.

50. The method of claim 16, wherein the PACAP analog, or a pharmaceutically acceptable salt thereof, is conjugated to a therapeutic or anticancer agent.

51. The method of claim 16, wherein the PACAP analog has an additive anticancer effect with one or more other anticancer agents.

52. The method of claim 16, wherein the subject is being treated with one or more anticancer agents for a hematopoietic cancer.

53. The method of claim 16, wherein the subject is being treated with one or more therapeutic or anticancer agents for a myeloproliferative disorder.

54. The method of claim 16, wherein the subject is being treated with one or more therapeutic or anticancer agents for multiple myeloma.

55. The method of any one of claims 16 to 54, wherein said subject is a mammal.

56. The method of claim 55, wherein said mammal is a human.

57. A method for the localization, diagnosis, or treatment of a disseminated cancer and metastatic tumor in a subject comprising administering an effective amount of a conjugate comprising one or more PACAP analogs of any one of claims 1 to 15 or a pharmaceutically acceptable salt thereof coupled to one or more radionuclides.

58. The method of claim 57, wherein said one or more PACAP analogs bind to one or more of PACAP/VIP receptors on the surface of one or more cells of the disseminated cancer or metastatic tumor.

59. The method of any one of claims 57 to 58, wherein said PACAP analog is selected from one or more of the following:
(SEQ ID NO: 4)
Iac Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 5)
Iaa Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 6)
Iaa Ser Asp Gly Ile Phe Thr Asp Ser Tyr Ser Arg
Tyr Arg Lys Ala Ala Ala Val Lys Lys Tyr Leu Ala
Ala Val Leu Gly Lys Arg Tyr Lys Gln Arg Val Lys
Asn D-Lys-NH₂;
(SEQ ID NO: 7)
Iaa D-Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 8)
Iac Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 9)
Iaa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 10)
Paa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 11)
Iaa D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 12)
Iaa D-Tyr Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 13)
Iaa D-Ala Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 14)
Iac D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂.

60. The method of any one of claims 57 to 59, wherein said disseminated cancer and metastatic tumor is a hematological cancer.

61. The method of claim 60, wherein said hematological cancer is a leukemia, lymphoma, or plasma cell dyscrasia.

62. The method of claims 57 to 61, wherein said PACAP analog binds to a target cell that is a component of a granuloma caused by one or more infectious agents or an autoimmune disease.

63. The method of any one of claims 57 to 62, wherein the subject is being treated with one or more of said conjugates for lymphoid or myeloid cancer.

64. The method of any one of claims 57 to 63, wherein the subject is being treated with one or more of said conjugates for multiple myeloma.

65. The method of any one of claims 57 to 64, wherein said subject is a mammal.

66. The method of claim 65, wherein said mammal is a human.

67. A method of producing a conjugate comprising coupling one or more radionuclides or small molecules to one or more PACAP analogs of any one of claims 1 to 15.

68. The method of claim 67, wherein said PACAP analog is selected from one or more of the following, or a pharmaceutically acceptable salt thereof:
(SEQ ID NO: 4)
Iac Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 5)
Iaa Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 6)
Iaa Ser Asp Gly Ile Phe Thr Asp Ser Tyr Ser Arg
Tyr Arg Lys Ala Ala Ala Val Lys Lys Tyr Leu Ala
Ala Val Leu Gly Lys Arg Tyr Lys Gln Arg Val Lys
Asn D-Lys-NH₂;
(SEQ ID NO: 7)
Iaa D-Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 8)
Iac Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 9)
Iaa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 10)
Paa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 11)
Iaa D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 12)
Iaa D-Tyr Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 13)
Iaa D-Ala Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 14)
Iac D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂.

69. The method of claim 67 or 68, wherein said radionuclide is selected from ¹¹C, ¹³N, ¹⁵O, ¹⁸F, ⁵²Fe, ⁵⁵Co, ⁶¹Cu, ⁶²Cu, ⁶⁴Cu, ⁶⁷Cu, ⁶⁷Ga, ⁶⁸Ga, ⁶²Zn, ⁶³Zn, ⁷⁰As, ⁷¹As, ⁷⁴As, ⁷⁶Br, ⁷⁹Br, ⁸²Rb, ⁸⁶Y, ⁸⁹Zr, ¹¹⁰In, ¹¹¹In, ¹²⁰I, ¹²³I, ¹²⁴I, ¹²⁵I, ¹³¹I, ¹²²Xe, ¹⁷⁵Lu, ¹⁵⁴Gd, ¹⁵⁵Gd, ¹⁵⁶Gd, ¹⁵⁷Gd, ¹⁵⁸Gd, ^{94m}Tc, ⁹⁴Tc, and ^{99m}Tc.

70. The method of any one of claims 67 to 69, wherein said small molecule is a therapeutic or anticancer agent.

71. The method of claim 70, wherein said therapeutic or anticancer agent is cisplatin, carboplatin, oxaliplatin, bleomycin, mitomycin C, calicheamicins, maytansinoids, auristatins, esperamicins, geldanamycin, doxorubicin, idarubicin, daunorubicin, epirubicin, busulfan, carmustine (BCNU), lomustine (CCNU), semustine, fotemustine, bendamustine, nimustine, thalidomide, lenalidomide, methotrexate, azathioprine, 6-mercaptopurine, fludarabine, 5-azacytidine, pentostatin (2′-deoxycoformycin), cytarabine (cytosine arabinoside), gemcitabine, 5-fluorouracil, hydroxyurea, elesclomol, etoposide, teniposide, amsacrine, mitoxantrone, camptothecin, topotecan, irinotecan, chlorambucil, cyclophosphamide, ifosfamide, melphalan, bortezomib, vincristine, vinblastine, vinorelbine, paclitaxel, docetaxel, iobitridol, iodipamide, iodixanol, iohexol, iomeprol, iopamidol, iopentol, iopromide, iotrolan, ioversol, ioxilan, iothalamate, ioxithalamate, ioxaglate, metrizamide, acetrizoate, metrizoate, diatrizoate, cyclosporine A, tacrolimus (FK506), sirolimus (rapamycin), everolimus, temsirolimus, zotarolimus, or biolimus.

72. A method for targeted delivery of a therapeutic or anticancer agent to a specific cell or tissue of a subject comprising administering to said subject an effective amount of a conjugate comprising one or more PACAP analogs of any one of claims 1 to 15, or a pharmaceutically acceptable salt thereof, coupled to one or more small molecules.

73. The method of claim 72, wherein said one or more PACAP analogs of any one of claims 1 to 15 bind to one or more PACAP/VIP receptors on the surface of said cell or tissue and the conjugate enters the interior of the cell or tissue by receptor-mediated endocytosis.

74. The method of claim 73, wherein said PACAP analog is selected from one or more of the following, or a pharmaceutically acceptable salt thereof:
(SEQ ID NO: 4)
Iac Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 5)
Iaa Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH2;
(SEQ ID NO: 6)
Iaa Ser Asp Gly Ile Phe Thr Asp Ser Tyr Ser Arg
Tyr Arg Lys Ala Ala Ala Val Lys Lys Tyr Leu Ala
Ala Val Leu Gly Lys Arg Tyr Lys Gln Arg Val Lys
Asn D-Lys-NH₂;
(SEQ ID NO: 7)
Iaa D-Ser Asp Gly Ile Phe Thr Asp Ser Tyr Ser Arg
Tyr Arg Lys Gln Met Ala Val Lys Lys Tyr Leu Ala
Ala Val Leu Gly Lys Arg Tyr Lys Gln Arg Val Lys
Asn Lys-NH₂;
(SEQ ID NO: 8)
Iac Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 9)
Iaa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 10)
Paa Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 11)
Iaa D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 12)
Iaa D-Tyr Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
(SEQ ID NO: 13)
Iaa D-Ala Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂;
and
(SEQ ID NO: 14)
Iac D-Ser Asp Gly Ile Phe Thr Asp Ser Tyr Ser Arg
Tyr Arg Lys Gln Met Ala Val Lys Lys Ala Leu Ala
Ala Val Leu Gly Lys Arg Tyr Lys Gln Arg Val Lys
Asn Lys-NH₂.

75. The method of any one of claims 72 to 74, wherein said subject has a disease.

76. The method of claim 75, wherein said disease is selected from an age-related neurodegenerative disease, a central nervous system disorder, Huntington's disease or other CAG codon repeat expansion disease, a retinal disease, an autoimmune disease, graft-versus-host disease, keratoconjunctivitis sicca caused by aging, autoimmune diseases or keratorefractive surgery, type II diabetes, sepsis caused by a bacteria and/or a virus, an acute or chronic cardiovascular disease, an acute or chronic renal diseases, a genetic disorder caused by a premature in-frame stop codon, an acute or chronic pulmonary disease, systemic hypertension, a hematological cancer, an eating disorder, an acute or chronic liver disease, osteoporosis, pre-eclampsia, cell and solid organ transplantation, a cognitive disorder, acquired immunodeficiency syndrome (AIDS) dementia complex, and aging of the central nervous system.

77. The method of claim 76, wherein:
i) said age-related neurodegenerative disease is selected from Alzheimer's disease, Parkinson's disease and amyotrophic lateral sclerosis;
ii) said central nervous system disorder is caused by stroke, heart attack or blunt force trauma, wherein preferably said blunt force trauma is a concussion or spinal cord trauma;
iii) said retinal disease is ischemia/reperfusion injury, non-infectious uveitis, diabetic retinopathy, macular degeneration, or glaucoma,
iv) said autoimmune disease is rheumatoid arthritis, Crohn's disease, ulcerative colitis, scleroderma, Sjögren's disease, idiopathic membranous nephropathy, Goodpasture's disease, autoimmune hepatitis, autoimmune myocarditis, myasthenia gravis, multiple sclerosis, Guillain-Barré syndrome, type I diabetes, Hashimoto's thyroiditis, Graves' disease, pemphigus vulgaris, or systemic lupus erythematosus;
v) said sepsis is caused by a bacteria or a virus;
vi) said acute or chronic cardiovascular disease is myocardial infarction, atherosclerosis, or restenosis, restenosis, or a drug-induced cardiomyopathy;
vii) said acute or chronic renal disease is ischemia/reperfusion injury, nephritis, or drug-induced nephrotoxicity;
viii) said acute or chronic pulmonary disease is asthma, chronic obstructive pulmonary disease, cystic fibrosis, or pulmonary arterial hypertension;
ix) said hematological cancer is a lymphoid or myeloid hematopoietic cancer, wherein preferably said lymphoid or myeloid hematopoietic cancer is a leukemia, a lymphoma, or a plasma cell dyscrasia;
x) said acute or chronic liver disease is ischemia/reperfusion injury, hepatitis, or fatty liver;
xi) said genetic disorder caused by a premature in-frame stop codon is cystic fibrosis, Duchenne muscular dystrophy, Krabbe's disease (globoid cell leukodystrophy), Hurler's syndrome, retinitis pigmentosa, ataxia telangiectasia, nephropathic cystinosis, or polycystic kidney disease; or
xii) said keratoconjunctivitis sicca is caused by aging, an autoimmune disease, corneal transplantation, or keratorefractive surgery.

78. The method of claim 76 or 77, wherein said disease causes injury to one or more major organs of the body of said subject due to treatment with a therapeutic or anticancer agent other than said PACAP analog, trauma, or acute or chronic disease.

79. The method of any one of claims 72 to 78, wherein the conjugate, or a pharmaceutically acceptable salt thereof, binds to one or more PACAP/VIP receptors and/or reduces one or more injuries to one or more major organs of the body of said subject due to treatment with a therapeutic or anticancer agent other than said PACAP analog, trauma, or acute or chronic disease.

80. The method of claim 78 or 79, wherein said one or more major organs of the body comprise nervous system, brain, spinal cord, heart, lung, kidneys, liver, pancreas, gall bladder, gastrointestinal tract, adrenal gland, thymus, spleen, lymph nodes, breast, ovary, testes, cornea, and prostate, preferably wherein one or more major organs of the body comprise nervous system, heart, lung, kidneys, liver, cornea, and gastrointestinal tract.

81. The method of any one of claims 75 to 78, wherein said disease is cancer or an autoimmune disease.

82. The method of any one of claims 72 to 81, wherein said small molecule is therapeutic agent or anticancer agent.

83. The method of claim 82, wherein said therapeutic or anticancer agent is cisplatin, carboplatin, oxaliplatin, bleomycin, mitomycin C, calicheamicins, maytansinoids, auristatins, esperamicins, geldanamycin, doxorubicin, idarubicin, daunorubicin, epirubicin, busulfan, carmustine (BCNU), lomustine (CCNU), semustine, fotemustine, bendamustine, nimustine, thalidomide, lenalidomide, methotrexate, azathioprine, 6-mercaptopurine, fludarabine, 5-azacytidine, pentostatin (2′-deoxycoformycin), cytarabine (cytosine arabinoside), gemcitabine, 5-fluorouracil, hydroxyurea, elesclomol, etoposide, teniposide, amsacrine, mitoxantrone, camptothecin, topotecan, irinotecan, chlorambucil, cyclophosphamide, ifosfamide, melphalan, bortezomib, vincristine, vinblastine, vinorelbine, paclitaxel, docetaxel, iobitridol, iodipamide, iodixanol, iohexol, iomeprol, iopamidol, iopentol, iopromide, iotrolan, ioversol, ioxilan, iothalamate, ioxithalamate, ioxaglate, metrizamide, acetrizoate, metrizoate, diatrizoate, cyclosporine A, tacrolimus (FK506), sirolimus (rapamycin), everolimus, temsirolimus, zotarolimus, or biolimus.

84. The method of any one of claims 72 to 82, wherein said small molecule is anti-inflammatory agent and said subject is being treated for rheumatoid arthritis.

85. The method of any one of claims 72 to 82, wherein said small molecule is a anticancer agent and said subject is being treated for multiple myeloma.

86. The method of any one of claims 72 to 85, wherein said subject is a mammal.

87. The method of claim 86, wherein said mammal is a human.

88. A method for detecting a granuloma in subject comprising administering to said subject an effective amount of the PACAP analog of any one of claims 1 to 15, or a pharmaceutically acceptable salt thereof, conjugated to a radionuclide.

89. The method of claim 88, wherein said radionuclide is ¹¹C, ¹³N, ¹⁵O, ¹⁸F, ⁵²Fe, ⁵⁵Co, ⁶¹Cu, ⁶²Cu, ⁶⁴Cu, ⁶⁷Cu, ⁶⁷Ga, ⁶⁸Ga, ⁶²Zn, ⁶³Zn, ⁷⁰As, ⁷¹As, ⁷⁴As, ⁷⁶Br, ⁷⁹Br, ⁸²Rb, ⁸⁶Y, ⁸⁹Zr, ¹¹⁰In, ¹¹¹In, ¹²⁰I, ¹²³I, ¹²⁴I, ¹²⁵I, ¹³¹I, ¹²²Xe, ¹⁷⁵Lu, ¹⁵⁴Gd, ¹⁵⁵Gd, ¹⁵⁶Gd, ¹⁵⁷Gd, ¹⁵⁸Gd, ^{94m}Tc, ⁹⁴Tc, and ^{99m}Tc.

90. The method of claim 88 or 89, wherein said subject has an infectious or autoimmune disease.

91. The method of any one of claims 88 to 90, wherein the polypeptide is a PACAP analog capable of binding to one or more of the PACAP/VIP receptors on the surface of target cells.

92. The method of any one of claims 88 to 91, wherein said subject is being treated for tuberculosis.

93. The method of any one of claims 88 to 92, wherein said subject is being treated with one or more of said conjugates comprising an imaging agent for tuberculosis.

94. The method of claim 92, wherein said subject is being treated with ^{99m}Tc-isonicotinylhydrazine (INH).

95. The method of any one of claims 88 to 91, wherein the subject is being treated for Crohn's disease.

96. The method of claim 95, wherein said subject is being treated with one or more of said conjugates comprising an imaging agent for Crohn's disease.

97. The method of any one of claims 88 to 96, wherein said subject is a mammal.

98. The method of claim 97, wherein said subject is a human.

99. The method of any one of claims 88 to 98, wherein said subject is being treated with a primary therapeutic selected from one or more of cisplatin, carboplatin, oxaliplatin, bleomycin, mitomycin C, calicheamicins, maytansinoids, auristatins, esperamicins, geldanamycin, doxorubicin, idarubicin, daunorubicin, epirubicin, busulfan, carmustine (BCNU), lomustine (CCNU), semustine, fotemustine, bendamustine, nimustine, thalidomide, lenalidomide, methotrexate, azathioprine, 6-mercaptopurine, fludarabine, 5-azacytidine, pentostatin (2′-deoxycoformycin), cytarabine (cytosine arabinoside), gemcitabine, 5-fluorouracil, hydroxyurea, elesclomol, etoposide, teniposide, amsacrine, mitoxantrone, camptothecin, topotecan, irinotecan, chlorambucil, cyclophosphamide, ifosfamide, melphalan, bortezomib, vincristine, vinblastine, vinorelbine, paclitaxel, docetaxel, iobitridol, iodipamide, iodixanol, iohexol, iomeprol, iopamidol, iopentol, iopromide, iotrolan, ioversol, ioxilan, iothalamate, ioxithalamate, ioxaglate, metrizamide, acetrizoate, metrizoate, diatrizoate, cyclosporine A, tacrolimus (FK506), sirolimus (rapamycin), everolimus, temsirolimus, zotarolimus, or biolimus.

100. The method of claim 99, wherein said methotrexate, carmustine, vincristine, paclitaxel, or thalidomide is used in adjunctive therapy.

101. The method of claim 99, wherein said subject has a lymphoid or myeloid cancer.

102. A PACAP analog comprising an amino acid selected from a group consisting of Iaa, Iac, Ica, and Paa at position 1.

103. A method for treating, managing, or preventing a disease selected from an age-related neurodegenerative disease, a central nervous system disorder, Huntington's disease or other CAG codon repeat expansion disease, a retinal disease, an autoimmune disease, graft-versus-host disease, keratoconjunctivitis sicca caused by aging, autoimmune diseases or keratorefractive surgery, type II diabetes, sepsis caused by a bacteria and/or a virus, an acute or chronic cardiovascular disease, an acute or chronic renal disease, a genetic disorder caused by a premature in-frame stop codon, an acute or chronic pulmonary disease, systemic hypertension, a hematological cancer, a granuloma, an eating disorder, an acute or chronic liver disease, osteoporosis, pre-eclampsia, cell and solid organ transplantation, a cognitive disorder, acquired immunodeficiency syndrome (AIDS) dementia complex, and aging of the central nervous system comprising administering to a subject in need thereof an effective amount of one or more of the PACAP analogs of any one of claims 1 to 3 or a pharmaceutically acceptable salt thereof.

104. A method for the localization, diagnosis, or treatment of a disseminated cancer and metastatic tumor in a subject comprising administering an effective amount of a conjugate comprising one or more of the PACAP analogs of any one of claims 1 to 3 or a pharmaceutically acceptable salt thereof coupled to one or more radionuclides.

105. A method for targeted delivery of a therapeutic or anticancer agent to a specific cell or tissue of a subject comprising administering to said subject an effective amount of a conjugate comprising one or more of the PACAP analogs of any one of claims 1 to 3 or a pharmaceutically acceptable salt thereof, coupled to one or more small molecules.

106. A method for detecting a granuloma in subject comprising administering to said subject an effective amount of the PACAP analogs of any one of claims 1 to 3 or a pharmaceutically acceptable salt thereof, conjugated to a radionuclide.
